(19) United States
(12) Patent Application Publication
(10) Pub. No.: US 2006/0073591 A1
(43) Pub. Date: Apr. 6, 2006

(54) Cell culture media

(76) Inventors: M. Abi Abitorabi, Morgan Hill, CA (US); Michael N. Guerini, Ong, NE (US); Stephen A. Taylor, Santa Cruz, CA (US); Guadalupe G. Hernandez, San Jose, CA (US); Christian C. Simonsen, Carmel, CA (US)

Correspondence Address: KING & SPALDING LLP, 191 PEACHTREE STREET, N.E., 45TH FLOOR, ATLANTA, GA, 30303-1763, US

(21) Appl. No.: 11/032,558

(22) Filed: Jan. 10, 2005

Related U.S. Application Data
(60) Provisional application No. 60/535,580, filed on Jan. 9, 2004.
(60) Provisional application No. 60/568,084, filed on May 4, 2004.

(51) Int. Cl. C12P 21/04; C12N 5/00

(52) U.S. Cl. 435/404; 435/70.21

(57) ABSTRACT

The invention is a cell culture medium that can include reduced or no serum and that enhances the performance of serum-free media for cell culture. The medium supports the growth of cells for both small scale and large scale propagation of cells. The invention also includes a method of cultivating cells using the cell culture medium of the present invention.

This application claims priority to U.S. Ser. No. 60/535,580 filed Jan. 9, 2004, and U.S. Ser. No. 60/568,084 filed May 4, 2004, the contents of which are herein incorporated by reference.

### FIELD OF THE INVENTION

The invention is a cell culture medium that can include reduced or no serum and that enhances the performance of serum-free media for cell culture. The medium supports the growth of cells for both small scale and large scale propagation of cells. The invention also includes a method of cultivating cells using the cell culture medium of the present invention.

### BACKGROUND OF THE INVENTION

Biotechnology drugs are medicines, such as therapeutic proteins (monoclonal antibodies, blood proteins and enzymes) that are produced by living organisms to fight disease. Unlike other medicines, biotech drugs are generally not produced synthetically, but are usually produced through microbial fermentation in mammalian cell culture. They can be more difficult, time-consuming and expensive (at least $250 million in production facility costs alone) to produce than synthetic drugs.

It is estimated that there are more than 370 new biotechnology medicines in the pipeline. Producing biotech drugs is a complicated and time-consuming process. Cells must be grown in large stainless-steel fermentation vats under strictly maintained and regulated conditions. In some cases the proteins are secreted by the cells; in other cases the cells must be broken open so the protein can be extracted and purified. Once the method is tested, devised and scaled up, the biotech medicines can be produced in large batches. This is done by growing host cells that have been transformed to contain the gene or antibody of interest in carefully controlled conditions in large stainless-steel tanks. The cells are kept alive and stimulated to produce the target proteins through precise culture conditions that include a balance of temperature (which can often vary by no more than one degree Celsius), oxygen, acidity (if pH levels change by even a small fraction, cells can easily die), media components and other variables. After careful culture in the appropriate media or serum (the duration varies depending on the protein produced and the nature of the organism), the proteins are isolated from the cultures, stringently tested at every step of purification, and formulated into pharmaceutically-active products. All of these procedures are in strict compliance with Food and Drug Administration (FDA) regulations. (htp://www.bio.org/pmp/factsheet1.asp, “A Brief Primer on Manufacturing Therapeutic Proteins”).

There are many varied types of cell culture media that can be used to support cell viability, for example DMEM medium (H. J. Morton, In Vitro, 6, 89/1970), F12 medium (R. G. Ham, Proc. Natl. Acad. Sci. USA, 53, 288/1965) and RPMI 1640 medium (J. W. Goding, J. Immunol. Methods, 39, 285/1980; JAMA 199, 519/1957). Such media (often called “basal media”), however, are usually seriously deficient in the nutritional content required by most animal cells. Typically, serum must be added to the basal media to overcome these deficiencies. Generally, fetal bovine serum (FBS), horse serum or human serum is used in significant concentrations.

While the use of FBS is desirable, and often necessary, for proper cell growth, it has several disadvantages. It is a relatively expensive material, and its use greatly increases the cost of cell culture. In addition, it is difficult to obtain serum with consistent growth characteristics. Further, the biochemical complexity of FBS can complicate the downstream processing of the proteins of interest, therefore raising the production costs.

The revolution in cell culture techniques is prompting research on commercial scale processes. Research and development spending, along with increasing competition, new products, the international marketplace and a changing customer base (due to the use of serum free media) are contributing to the creation of a new kind of market in this field. Serum-free medium is an excellent alternative to standard serum-containing media for the cultivation of cells. It has several advantages, which include better definition of the composition, reduced contamination and lower cost.

A serum-free medium having cultivation ability comparable to that of the conventional serum-containing medium has long been sought. There is a continuing need in the art for cell culture media that are simple to prepare, economical, and that provide all of the necessary nutrients and growth factors, at suitable concentrations, to optimize the growth of the cells.

One strategy to develop serum-free media has been to supplement the basal media with appropriate nutrients to avoid the addition of FBS, without sacrificing cell growth and/or protein production. Examples of such components include bovine serum albumin (BSA) or human serum albumin (HSA); certain growth factors derived from natural (animal) or recombinant sources, including epidermal growth factor (EGF) or fibroblast growth factor (FGF); lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth, C., in: Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc., pp. 23-68 (1984), and by Gospodarowicz, D., Id., at pp 69-86 (1984)).

It is known that cholesterol and cholesterol-containing fractions obtained from bovine serum are useful to promote the growth of various organisms. J. Bacteriol., Vol. 135, pp. 818-827 (1978) describes the use of a cholesterol-containing bovine serum fraction in the growth of Mycoplasma pneumoniae and Mycoplasma arthritidis. J. Gen. Microbiology, Vol. 116, pp. 539-543 (1980) describes the use of USP cholesterol in the growth of Treponema hyodysenteriae. In Vitro, Vol. 17, No. 5, pp. 519-530 (1981) discloses that mixtures of high density lipoproteins and transferrin can be used to grow certain mammalian cells in the absence of serum.

U.S. Pat. No. 4,290,774 describes the production of a specific cholesterol-rich fraction from mammalian plasma or serum by a process that involves the step of treatment with an alkaline carbonate and an alkaline earth salt. Zeit. Klin. Chem. 6(3), pp. 186-190 (1968) describes the removal of certain lipoproteins from human serum by use of colloidal silicic acid.

U.S. Pat. No. 4,762,792 and European Patent No. EP0201800 disclose a process for isolating a cholesterol-rich fraction from mammalian blood plasma or serum using a silica adsorbant followed by several alkaline steps, which is useful as a growth medium ingredient, especially in cell culture.

U.S. Pat. No. 5,409,840 describes an improved process for the recovery of cholesterol rich fractions from mammalian serum or plasma. The process involves adsorbing the fraction on precipitated silica gel agglomerates which are then separated from the serum or plasma whereupon the adsorbed cholesterol rich fraction is eluted from the silica and recovered.

EX-CYTE® is a concentrated aqueous mixture of cholesterol, lipoproteins and fatty acids that is manufactured by Serologicals, Inc. using the process described in U.S. Pat. No. 4,762,792. EX-CYTE® is typically made from bovine serum.

In 1989, Hewlett et al. (Miles Science Journal 1989,11: 9-14) described the effects of the addition of EX-CYTE® to serum-free or low serum containing culture media on the growth of several cell types, including L929 cells, CHO-K1 cells, BHK-21 cells, AHT-107 hybridoma cells, mouse myeloma cells and monkey-fibroblast cells. The cells were grown in media containing several of the following components (the components varied depending on cell type): insulin, transferrin, selenite, bovine EX-CYTE®, human EX-CYTE®, freeze-dried EX-CYTE®, 1% FBS, selenite, human serum albumin (HAS) and/or trace elements.

In 1991, Guy Hewlett (Prod. Biol. Anim. Cells Cult. (1991) ESACT 10 Meet., 67-69) further described the effect of the addition of EX-CYTE®, to serum-free or low serum containing culture media on the growth of several cell types, including A431 human keratinocytes, L929 mouse fibroblast cells, 3T3 cells, X63 myeloma cells, AHT hybridoma cells and HeLa cells. The cells were grown in media containing 50/50 mix of Dulbecco's modification of Eagle's medium (DMEM) and Ham's nutrient solution F12 (F12) and several of the following additional components (the components varied depending on cell type): bovine insulin (10 mg/L), transferrin (10 mg/L), EX-CYTE®, lipoprotein/lipid (30 ug cholesterol/mL), albumin (200 mg/L), selenium (100 nmol/L) and/or 0.5% Fetal Calf Serum.

Savonniere et al. (Journal of Biotechnology 48 (1996) 161-173) described the effect of lipid supplementation of culture media on cell growth, antibody production, membrane structure and dynamics in two hybridoma cell lines (B9 cells (fusion of SP2/OAg14 cells with mouse Balb/C spleen cells) and A49 cells (fusion of SP2/O myeloma cells with mouse Balb/B lymphocytes)). B9 cells were grown in RPMI 1640 medium (Seromed. Strasbourg, France) supplemented with 100 UI/ml penicillin, 10 μg/ml streptomycin, 5% {v/v) fetal calf serum, 50 μM 2-mercaptoethanol, 100 UI/ml of recombinant human IL-6, and Ex-Cyte. A49 cells were grown in RPMI 1640 medium supplemented with antibiotics, 0.5% (v/v) FCS, 2% Ultroser HY and EX-CYTE®. They reported that the response to the two cell lines to EX-CYTE® was different, the addition of EX-CYTE® was without effect on the B9 cells, while A49 cells showed an increased growth rate.

PCT Publication No. WO 90/07007 filed by the United States of America discloses a serum free media for culturing animal epithelial cells, including human epithelial cells. The patent discloses a media with the following components: L-glutamine, 2 mM, Insulin, 10 ug/ml, Hydrocortisone, 0.2 uM, epidermal growth factor, 5.0 ng/ml, transferrin, 10 ug/ml, phosphoethanolamine, 0.5 uM, cholera toxin, 25 ng/ml, triiodothyronine, 10 nM, retinoic acid, 10 nM, ornithine, 2 mM, CaCl₂, 0.4 mM, Glucose, 2.0 mg/ml, bovine pituitary extract, 7.5 ug/ml, EX-CYTE® V, 312 ug/ml, FeSO₄≅7H₂O, 2.7 uM, ZnSO₄≅7H₂O, 0.5 uM, Na₂SeO₃, 3.0×10⁻⁸ M, MnCl₂≅4H₂O, 1.0 nM, Na₂SiO₃≅9H₂O, 5.0×10⁻⁷ M, (NH₄)₆ Mo₇O₂₄≅4H₂O, 1.0 nM, NH₄VO₃, 5.0 nM, NiSO₄≅6H₂O, 0.5 nM, SnCl₂≅2H₂O, 0.5 nM, and Gentamicin, 50 ug/ml.

U.S. Pat. No. 6,733,746 to Daley et al. and U.S. Publication No. 2004/0072349 filed by Daley et al. disclose a hematopoetic cell culture nutrient supplement. The supplement disclosed contains one or more antioxidants, one or more albumins or albumin substitutes, one or more lipid agents, one or more insulins or insulin substitutes, one or more transferrins or transferrin substitutes, one or more trace elements, and one or more glucocorticoids. The patent application specifically discloses formulations for culturing hematopoetic stem cells that contain, for example N-acetyl-L-cysteine, human serum albumin, Human EX-CYTE®, ethanolamine HCl, zinc insulin, human iron saturated transferrin, a Se⁴+salt, hydrocortisone, D,L-tocopherol acetate, 2-mercaptoethanol and/or glutamine.

U.S. Pat. No. 5,932,703 to ICOS Corporation describes purified and isolated nucleotide sequences encoding a human macrophage-derived chemokine (MDC) and methods for the recombinant production of the same. Transfected CHO cells were used to express MDC. The media used to culture the CHO cells contained P5 medium (which consists of various components including glutamine) containing 0.2% to 1.0% FBS, 3 g/l sodium bicarbonate, 2 ug/l sodium selenite, 1% soy bean hydrolysate, ferrous sulfate/EDTA solution, 1.45 ml/L EX-CYTE VLE solution, 10 ug/ml recombinant insulin, 0.1% pluronic F-68, 30 ug/ml glycine, 50 uM ethanolamine and 1 mM sodium pyruvate.

Gorfien et al. (Biotechnol. Prog. 2000, 16, 682-687) describe the growth of NSO hybridoma cells in a protein free, chemically defined media. The media contains CD Hybridoma Medium and lipoprotein supplements, including EX-CYTE VLE (Bayer, catalog number 81-129) at 1:300, 1:500, and 1:1000 dilutions. This reference teaches that Ex-Cyte did not support the long term growth of NSO cells.

U.S. Patent Publication No. 2003/0166146 to Lee et al. describes a myeloma line useful for manufacturing recombinant proteins in chemically defined media. The chemically defined media used to culture the myeloma cell line (C463A myeloma cell line, a spontaneous mutant cloned from a Sp2/0-Ag14 cell bank) contained IMDM, Primatone, Albumin, and Ex-Cyte.

U.S. Pat. No. 5,240,848 to Monsanto Company describes a cDNA sequence for human vascular permeability factor and methods to recombinantly produce the same. U-937 cells (a human cell line established from a diffuse histiocytic lymphoma, ATCC CRL 1593) were used to produce the vascular permeability factor protein. The cells were cultured in media that contained the following components: RPMI 1640, DME (high glucose), Ham's F12 in a 1:1:1 ratio, HEPES (25 mM, pH 7.10-7.15) glutathione (1 mM), ethanolamine (20 uM), selenium (30 nM) or 5200 ug/ml, NaHCO₃ (2 mM), CuSO₄ (5 nM), NH4 VOs (5 nM), ZnSO₄ (0.5 uM), MnSO₄ (0.5 nM), FeSO₄ (4 uM), bovine serum albumin, Miles “Pentex” (100 ug/ml), iron rich transferrin, Miles (5 ug/ml), bovine insulin (10 ug/ml), F-68 Pluracol (0.05% w/v) and 0.1% Ex-Cyte.

In 2002, Serologicals, Inc. published a Technical Bullitin disclosing increased protein yield in an antihuman IgGG monoclonal antibody-producing cell line. The publication disclosed the use of Ex-Cyte (30 ug cholesterol), human serum albumin (10 mg/ml), human APO-transferrin (2.5 ug/ml), insulin (5 ug.ml) and sodium selenite (10⁻⁷M).

It is an object of this invention to provide culture media that supports the growth of various cell types.

Another object of this invention is to provide culture media that are serum-reduced or serum-free to support the growth of various cell types.

A further object of the present invention is to provide cell culture media for use in either small-scale culture or large-scale commercial production of cells.

A still further object of this invention is to provide a cell culture medium that increases the yield of biological materials, for example, peptides, produced by the cells cultured in such media.

Another object of this invention is to provide a method of culturing cells in a suitable medium to allow cell growth.

A further object of this invention is to provide a method of culturing cells in a suitable medium for the production of biological materials of interest.

A still further object of the present invention is to provide methods to make cell culture media.

### SUMMARY OF THE INVENTION

The invention provides novel cell culture media compositions that include purified lipoprotein material that reduces or eliminates the use of serum or enhance the performance of serum-free media for cell culture. The invention also includes methods of culturing cells using the cell culture media compositions. The compositions and/or methods are useful in the culture of a variety of cell types, including, for example, hybridoma cells and/or cancer cells.

In a first embodiment, the cell culture composition can include (i) basal media; (ii) purified lipoprotein material; and/or (iii) a reduced concentration of serum, such as fetal bovine serum (FBS), relative to the use of serum alone to supplement basal media. Normally, basal media is supplemented with approximately 10% (alternatively by weight or volume) or more serum, such as FBS. In this embodiment, basal media can be supplemented with less than 10% serum, such as FBS, and/or more particularly, approximately 9.5, 9, 8.5, 8, 7.5, 7, 6.5, 6, 5.5, 5, 4.5, 4, 3.5, 3, 2.5, 2, 1.5, 1, 0.5 or 0.25% serum, such as FBS, in combination with a purified lipoprotein material. In one embodiment, between 0.1 and 5% purified lipoprotein material can be used in the composition. In another embodiment, up to approximately 0.2, 0.3, 0.4, 0.5, 0.6, 0.7, 0.75, 0.8, 0.9, 1.0, 1.5 or 2% purified lipoprotein material is used. In another embodiment, approximately 0.2, 0.3, 0.4, 0.5, 0.6, 0.7, 0.75, 0.8, 0.9, 1.0, 1.5 or 2% purified lipoprotein material can be used.

In embodiments of the present invention, the purified lipoprotein material can be derived from serum or plasma obtained from a mammal. In one embodiment, the purified lipoprotein material can be a cholesterol-rich fraction, optionally in association with low density lipoprotein (LDL) and/ or high density lipoprotein (HDL). In another embodiment, the purified lipoprotein material can be derived from bovine, horse, sheep, pig or human serum or plasma. In a further embodiment, the purified lipoprotein material can contain cholesterol, such as approximately 5-15, more particularly, 9, 10 or 11, grams per liter of cholesterol as determined, for example, by enzymatic assay; protein, such as approximately 10-20, more particularly, 13, 14, 15, 16, 17 or 18, grams per liter of protein, and/ or approximately 0-10, particularly, 0-6, EU endotoxin per milligram of cholesterol, such as determined, for example, by limulus amebocyte lysate. In another embodiment, the pH of the purified lipoprotein material can be between approximately 7 and 8, more particularly, 7.0-8.4. In other embodiments, the purified lipoprotein material does not contain detectable levels of contaminants or undesired materials, for example, immunoglobulin G (IgG), microbes, mycoplasm, and/or viral agents.

In further embodiments, the purified lipoprotein material can be produced by contacting the plasma or serum or derivative thereof with an adsorbant, such as silica. In a specific embodiment, the purified lipoprotein material produced according to the following process: (a) contacting a liquid cholesterol-containing plasma or serum or fraction thereof with a silica adsorbent to adsorb the cholesterol-rich fraction; (b) separating the adsorbed cholesterol-rich fraction from the remaining liquid plasma or serum; (c) freezing and/or thawing the adsorbed cholesterol-rich fraction; (d) eluting the adsorbed cholesterol-rich fraction at a pH from 9.0 to 11.5; (e) either before or after step (f) and/or prior to step (g) adjusting the pH of the cholesterol-rich solution to a value in the range from 11.0 to 13.0; (f) concentrating the cholesterol-rich solution by ultrafiltration; (g) dialyzing the concentrated cholesterol-rich solution sequentially against sodium carbonate and/or water; (h) further concentrating the dialyzed cholesterol-rich solution by ultrafiltration; (i) adjusting the pH of the concentrated cholesterol-rich solution to a value in the range from 7.0 to 11.0; (j) heating the concentrated cholesterol-rich solution at 50° to 100° C. for 30 minutes to 24 hours; (k) recovering therefrom a purified cholesterol-rich fraction, such as described in detail in U.S. Pat. No. 4,762,792, including but not limited to the product EX-CYTE®.

In another embodiment, the composition can include (i) basal media; (ii) purified lipoprotein material; (iii) insulin; (iv) albumin; (v) sodium selenite; and/or (vi) transferrin. In one embodiment, the composition can include approximately 2, 2.5, 3, 3.5, 4, 4.5, 5, 5.5, 6, 6.5, 7, 7.5, 8, 8.5 or 9 mg/ml albumin, such as bovine serum albumin (BSA); at least 3.0, 3.5, 4.0, 4.5, 5.0, 5.5, 6.0, 7.0, 10, 15 or 20 ug/ml transferrin; approximately 5.5, 6.0, 6.5, 7.0, 7.5, 8.0, 8.5, 9.0, 9.5, 10, 10.5, 11, 11.5, 12, 15 or 20 ug/ml insulin; approximately 1, 2, 3, 2.5, 3, 3.5, 4, 4.5, 5, 5.5, 6, 6.5, 6.7, 7.0, 7.5, 8.0, 9.0, 10, 15 or 20 ug/L sodium selenite; and/or 0.2, 0.3., 0.4, 0.5, 0.6, 0.7, 0.75, 0.8, 0.9, 1.0, 1.5 or 2% purified lipoprotein material in basal media. In one specific embodiment, the cell culture media can include approximately 4 mg/ml BSA; approximately 5.5 ug/ml transferrin; approximately 10 ug/ml insulin; approximately 6.7, ug/L sodium selenite; and/or approximately 0.75% purified lipoprotein material in basal media.

In another embodiment, the composition can include (i) basal media; (ii) purified lipoprotein material (iii) glutamine; (iv) albumin; (v) insulin; (vi) transferrin; and/or (vii) ethanolamine. In one embodiment, the composition can include approximately 1, 2, 3, 4, 5, 6, 7, 8, 9, or 10 mM glutamine; approximately 0.1, 0.2, 0.3, 0.4, 0.5, 0.6, 0.7, 0.8, 0.9, 1, 2, 3, 4, 5, 6, 7, or 8% albumin, such as bovine serum albumin (BSA); approximately 1, 2, 3, 4, 5, 6, 7, 8, 9, 9.5, 10, 10.5, 11, 11.5, 12, 13, 14, 15, 16, 17, 18, 19, 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L transferrin; approximately 1, 2, 3, 4, 5, 6, 7, 8, 8.5, 9, 9.5, 10, 10.5, 11, 11.5, 12, 13, 14, 15, 16, 17, 18, 19 or 20 μM ethanolamine and/or approximately 0.2, 0.3, 0.4, 0.5, 0.6, 0.7, 0.8, 0.9, 1, 1.5, 2, 2.5, 3, 3.5, 4, or 5.0 % purified lipoprotein material in basal media. In one specific embodiment, the invention can include approximately 4 mM glutamine; approximately 0.5% BSA; approximately 10 mg/L insulin; approximately 1 mg/L transferrin; and/or approximately 10 molar ethanolamine, and/or approximately 2% purified lipoprotein material in basal media.

In another embodiment, the composition can include (i) basal media; (ii) purified lipoprotein material; (iii) glutamine; (iv) albumin; (v) insulin; and/or (vi) transferrin. In one embodiment, the composition can include approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% albumin, such as bovine serum albumin (BSA); approximately 1, 2, 3, 4, 5, 6, 7, 8, 9, 9.5, 10, 10.5, 11, 11.5, 12, 13, 14, 15, 16, 17, 18, 19, or 20 mg/L insulin; and/ or approximately 0.1, 0.2, 0.3, 0.4, 0.5, 0.6, 0.7, 0.8, 0.9, 0.95, 1, 1.5, 2, 2.5, 3, 4, 5, 6, or 7 mg/L transferrin. In one specific embodiment, the composition can include approximately 4 mM glutamine, approximately 0.1% purified lipoprotein material, approximately 1% BSA, approximately 10 mg/L insulin, and/or approximately 1 mg/L transferrin in basal media.

In another embodiment, the composition can include (i) basal media; (ii) purified lipoprotein material; (iii) glutamine; (iv) albumin; (v) insulin; and/or (vi) transferrin; and/or (vii) peptone. In one embodiment, the composition can include approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% albumin, such as bovine serum albumin (BSA); approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L transferrin; and/or approximately 0.01, 0.02, 0.03, 0.04, 0.05, 0.06, 0.07, 0.08, 0.09, 0.1, 0.2, 0.3, 0.4, 0.5, 1, 2 or 3% peptone. In one specific embodiment, the composition can include approximately 4 mM glutamine, approximately 0.1% purified lipoprotein material, approximately 1% BSA, approximately 10 mg/L insulin, approximately 1 mg/L transferrin, and/or approximately 0.1% peptone in basal media.

In embodiments of the present invention, the peptone or peptone mixture is a protein hydrolysate, which is obtained from hydrolyzed animal or plant protein. The peptones can be derived from animal by-products from slaughter houses, purified gelatin, or plant material. The protein from the animal or plant sources can be hydrolyzed using acid, heat or various enzyme preparations. Peptone mixtures that can be used include spy peptone, “Primatone RL” and/or “Primatone HS”, both of which are commercially available (Sheffield, England/or; Quest International (IPL:5X59051), PRIMATONE® RL).

In another embodiment, the composition can include (i) basal media; (ii) purified lipoprotein material; (iii) glutamine; (iv) albumin; (v) insulin; (vi) transferrin; and/or (vii) fetuin (such as Pedersen). In one embodiment, the composition can include approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% albumin, such as bovine serum albumin (BSA); approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L transferrin; and/or approximately 2, 3, 4, 5, 6, 7, 8, 9, 10, 10.5, 11, 11.5, 12, 12.5, 13, 13.5, 14, 14.5, 15, 16, 17, 18, 19, 20 μg/ml of fetuin. In one specific embodiment, the composition of the present invention can include approximately 4mM glutamine, approximately 0.1% purified lipoprotein material, approximately 1% BSA, approximately 10 mg/L insulin, approximately 1 mg/L transferrin, and/or approximately 12.5 μg/ml fetuin (such as Pedersens) in basal media.

In another embodiment, the composition can include (i) basal media; (ii) purified lipoprotein material; (iii) glutamine; (iv) albumin; (v) insulin; (vi) transferrin; and/or (vii) vitamin E. In one embodiment, the composition can include approximately 1, 2, 3, 4, 5, 6, 7, 8, 9, or 10 mM glutamine; approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% albumin, such as bovine serum albumin (BSA); approximately 1, 2, 3, 4, 5, 6, 7, 8, 9, 9.5, 10, 10.5, 11, 11.5, 12, 13, 14, 15, 16, 17, 18, 19, or 20 mg/L insulin; approximately 0.1, 0.2, 0.3, 0.4, 0.5, 0.6, 0.7, 0.8, 0.9, 0.95, 1, 1.5, 2, 2.5, 3, 4, 5, 6, or 7 mg/L transferrin; and/or approximately 1, 2, 3, 4, 5, 6, 7, 8, 9, or 10 micromolar vitamin E. In one specific embodiment, the composition of the present invention can include approximately 4 mM glutamine, approximately 0.1% purified lipoprotein material, approximately 1% BSA, approximately 10 mg/L insulin, approximately 1 mg/L transferrin, and/or approximately 5 μM vitamin E in basal media.

In a preferred embodiment of the invention, the composition can include approximately 4 mM glutamine, approximately 0.1% purified lipoprotein material, approximately 1% BSA, approximately 10 mg/L insulin, approximately 1 mg/L transferrin, approximately 0.1% peptone, approximately 12.5 μg/mL fetuin (such as Pederson), and/or approximately 5 μM vitamin E.

In a further embodiment, the composition can include (i) serum free media and/or (ii) purified lipoprotein material. In one embodiment, the serum free media is one of the media listed in Table 1. In another embodiment, the serum free media is either Hybridoma Media, animal component free or Ex-Cell (JRH Biosceinces, Inc.). In another embodiment, approximately 0.2, 0.3., 0.4, 0.5, 0.6, 0.7, 0.75, 0.8, 0.9, 1.0, 1.5 or 2% purified lipoprotein material can be used.

In an additional embodiment, the composition can include (i) serum free media and/or (ii) purified lipoprotein material; and/or (iii) albumin. In one embodiment, the serum free media is one of the media listed in Table 1. In another embodiment, the serum free media is either Hybridoma Media, animal component free or Ex-Cell (JRH Biosceinces, Inc.). In a further embodiment, the composition can include approximately 0.1, 0.2, 0.3, 0.4, 0.5, 0.6, 0.7, 0.8, 0.9, 1, 2, 3, 4, 5, or 10% albumin, such as bovine serum albumin (BSA). In another embodiment, approximately 0.2, 0.3., 0.4, 0.5, 0.6, 0.7, 0.75, 0.8, 0.9, 1.0, 1.5 or 2% purified lipoprotein material is used. In a specific embodiment, the composition can include ExCell, 0.75% purified lipoprotein material ® and/or 0.5% BSA. In another specific embodiment, the composition can include Hybridoma Medium, Animal Component-free, 0.5% purified lipoprotein material and/or 0.2% BSA.

In another aspect of the present invention compositions are provided that are useful as a cell culture medium that serves to increase the yield of biological products, such as proteins, produced by the cells cultured in the media. In one embodiment, compositions can increase the yield of biological products at least 25%, 30%, 50%, 100%, 200% or 300%. In another embodiment, the biological products produced can be a peptide, such as a therapeutic or diagnostic peptide, polypeptide, protein, monoclonal antibody, immunoglobulin, cytokine (such as interferon), integrin, antigen, growth factor, cell cycle protein, hormone, neurotransmitter, receptor, fusion peptide, blood protein and/ or chimeric protein.

In a further aspect of the present invention, compositions are provided that are useful as a cell culture medium for a variety of cells. In one embodiment, the cell culture media of the present invention can be used for adherent cell culture. In another embodiment, the cell culture media described herein can be used for suspension cell culture. In other embodiments, the cell culture media described herein can be used as culture media for hybridoma cells, monoclonal antibody producing cells, virus-producing cells, transfected cells, cancer cells and/or recombinant peptide producing cells. In one embodiment, the compositions can be used to culture eukaryotic cells, such as plant and/or animal cells. The cells can be mammalian cells, fish cells, insect cells, amphibian cells or avian cells. Other types of cells can be selected from the group consisting of MK2.7 cells (ATCC Catalogue No. CRL1909, an anti-murine-VCAM IgG1 expressing hybridoma cell), HEK 293 cells, PER-C6 cells, CHO cells, COS cells, 5L8 hybridoma cells, Daudi cells, EL4 cells, HeLa cells, HL-60 cells, K562 cells, Jurkat cells, THP-1 cells, Sp2/0 cells; and/or the hybridoma cells listed in Table 2 or any other cell type disclosed herein or known to one skilled in the art.

Basal media can include, but are not limited to Dulbecco's Modified Eagle's Medium (DMEM), Minimal Essential Medium (MEM), Basal Medium Eagle (BME), RPMI 1640, F-10, F-12, alpha. Minimal Essential Medium (.alpha.MEM), Glasgow's Minimal Essential Medium (G-MEM), and/or Iscove's Modified Dulbecco's Medium.

The present invention also provides a method of cultivating eukaryotic cells including contacting the cells with the compositions that are useful as cell culture medium of the present invention and/or maintaining the cells under conditions suitable to support cultivation of the cells in culture. In a particular embodiment, the cells are cancer cells or hybridoma cells. In other embodiments, methods of cultivating tissue explants are cultures are provided including contacting the tissues with the cell culture media compositions described herein.

In one embodiment, the method includes contacting hybridoma cells with a composition including: (i) basal media; (ii) purified lipoprotein material isolated as; (iii) glutamine; (iv) albumin; (v) insulin; (vi) transferrin; and/or (vii) ethanolamine, and/or maintaining the hybridoma cells under conditions suitable to support cultivation of the hybridoma cells in culture.

In a specific embodiment, the method includes contacting hybridoma cells with a composition including (i) basal media; (ii) approximately 2% purified lipoprotein material; (iii) approximately 4 mM glutamine; (iv) approximately 0.5% BSA; (v) approximately 10 mg/L insulin; (vi) approximately 1 mg/L transferrin; (vii) approximately 10 μM ethanolamine.

In another embodiment, the present invention is a method of cultivating cancer cells by contacting the cells with compositions that are useful as cell culture medium of the present invention and/or maintaining the cancer cells under conditions suitable to support cultivation of the cancer cells in culture. In a specific embodiment, the method includes contacting cancer cells with a composition including (i) basal media; (ii) purified lipoprotein material; (iii) glutamine; (iv) albumin; (v) insulin; and/or (vi) transferrin.

In a specific embodiment, the method includes contacting cancer cells with a composition including (i) basal media; (ii) purified lipoprotein material; (iii) glutamine; (iv) albumin; (v) insulin; (vi) transferrin; and/or (vii) peptone.

In a further embodiment, the method includes contacting cancer cells with a composition including (i) basal media; (ii) purified lipoprotein material; (iii) glutamine; (iv) albumin; (v) insulin; (vi) transferrin; and/or (vii) fetuin. In a specific embodiment, fetuin protein can be Pedersen's fetuin.

In another embodiment, the method involves contacting cancer cells with a composition including (i) basal media; (ii) purified lipoprotein material; (iii) glutamine; (iv) albumin; (v) insulin; (vi) transferrin; and/or (vii) vitamin E.

In a specific embodiment, the method of the present invention involves contacting cancer cells with a composition including (i) basal media; (ii) purified lipoprotein material; (iii) glutamine; (iv) approximately 1% BSA; (v) insulin; (vi) transferrin; (vii) peptone; and/or (viii) fetuin (such as Pedersens).

In yet another embodiment, the method of the present invention involves contacting cancer cells with a composition including (i) basal media; (ii) purified lipoprotein material; (iii) glutamine; (iv) approximately 1% BSA; (v) insulin; (vi) transferrin; (vii) fetuin; and/or (viii) vitamin E.

In a specific embodiment, the method of the present invention involves contacting cancer cells with a composition including (i) basal media; (ii) purified lipoprotein material; (iii) glutamine; (iv) approximately 1% BSA; (v) insulin; (vi) transferrin; (vii) peptone; (viii) fetuin (such as Pedersens); and/or (ix) vitamin E.

In a particular embodiment, the method of the present invention involves contacting cancer cells with a composition including (i) basal media; (ii) approximately 0.1% purified lipoprotein material; (iii) approximately 4 mM glutamine; (iv) approximately 1% BSA; (v) approximately 10 mg/L insulin; (vi) approximately 1 mg/L transferrin; (vii) approximately 0.1% peptone; (viii) approximately 12.5 μg/ml fetuin (such as Pedersens); and/or (ix) approximately 5 μM vitamin E.

In other embodiments of the present invention, methods are provided for the cultivation of cells wherein the cells can be incubated in a serum-containing media, such as about 0.5, 1, 2, 3, 4, 5, or 10% serum, such as FBS, followed by transfer of the cells into a serum-rescued or serum-free media of the present invention. In one embodiment, the cells can be grown to confluence and/ or maintained in serum-containing media and then transferred to the serum-free or serum-reduced media of the present invention. In one embodiment, the cells can be transferred to the media described herein prior to the production of biological materials from the cells. In an alternative embodiment, the cells can be grown only in serum-free or serum-reduced media of the present invention.

The present invention also provides a kit for the cultivation of cells in vitro, the kit comprising the compositions of the present invention. In another embodiment, the kit can contain compositions of the present invention in combination with specific cell lines.

### BRIEF DESCRIPTION OF THE FIGURES

FIG. 1 demonstrates the cell growth comparisons of EX-CYTE®+2% FBS vs. 10% FBS vs. 2% FBS in DME/F12.

FIG. 2 illustrates the IgG1 antibody production comparisons of EX-CYTE®+2% FBS vs. 10% FBS vs. 2% FBS in DME/F12.

FIG. 3 demonstrates the cell growth comparisons of EX-CYTE®+BSA+Insulin+Transferrin+Sodium selenite (ITS) vs. 10% FBS in DMEM.

FIG. 4 shows the IgG1 antibody production comparisons of EX-CYTE®+BSA+Insulin+Transferrin+Sodium selenite (ITS) vs. 10% FBS in DMEM.

FIG. 5 illustrates cell growth comparisons of EX-CELL™ 620 vs. EX-CELL™ 620+EX-CYTE® and BSA.

FIG. 6 demonstrates the IgG1 antibody production comparisons of EX-CELL™ 620 vs. EX-CELL™ 620+EX-CYTE® and BSA.

FIG. 7 shows the cell growth comparisons of Hybridoma Medium, Animal Component-free vs. Hybridoma Medium, Animal Component-free+EX-CYTE® and BSA.

FIG. 8 illustrates the IgG1 antibody production comparison of Hybridoma Medium, Animal Component-free vs. Hybridoma Medium, Animal Component-free EX-CYTE® and BSA.

FIG. 9 depicts the results of the range find experiments to identify optimal concentrations of BSa and Ex-Cyte to enhance the growth of K562 cells.

FIG. 10 depicts the results of the refined range find experiments to identify optimal concentrations of BSA and Ex-Cyte to enhance the growth of K562 cells.

FIG. 11 demonstrated the results of a comparison study of the effects of Soy Peptone substituted for Primatone RL in the XCF-2 formulation on the growth of K562 cells.

FIG. 12 demonstrates the performance of the XCF-2 media compared with the benchmark of 10% Fetal Bovine Serum on cell growth of K562 cells.

FIG. 13 shows the levels of expression of the CD32 marker in K562 cells grown in different medias. Cells were grown in either 0.5% FBS or 10% FBS. In column 1, the cells were grown adapted to 0.5% FBS and maintained in that level for the experiment. In column 2 and 3, cells were grown in 10% FBS and then subsequently cultured in 10% FBS (column 2) or XCF2 (column 3) for the experiment. In column 4 and 5, cells were adapted to growth in 0.5% FBS and then subsequently cultured in 10% FBS (column 4) or XCF2 (column 5) for the experiment.

### DETAILED DESCRIPTION

Cultured cells are widely used in the biopharmaceutical industry for the production of biopharmaceuticals, such as vaccines, proteins, peptides and monoclonal antibodies. As scientists and cell culturists continue to strive maximize product yield and minimize costs, care selection of media and supplements is critical. The invention is a composition that is useful as a cell culture media that can include reduced or no serum or enhances the performance of serum-free media for cell culture.

The invention provides novel cell culture media compositions that include purified lipoprotein material to reduce or eliminate the use of serum or enhance the performance of serum-free media for cell culture. The invention also includes methods of culturing cells using the cell culture media compositions. The compositions and/or methods are useful in the culture of a variety of cell types, including, for example, hybridoma cells and/or cancer cells.

In a first embodiment, the cell culture composition can include (i) basal media; (ii) purified lipoprotein material; and/or (iii) a reduced concentration of serum, such as fetal bovine serum (FBS), relative to the use of serum alone to supplement basal media. In another embodiment, the composition can include (i) basal media; (ii) purified lipoprotein material; (iii) insulin; (iv) albumin; (v) sodium selenite; and/or (vi) transferrin. In a further embodiment, the composition can include (i) basal media; (ii) purified lipoprotein material (iii) glutamine; (iv) albumin; (v) insulin; (vi) transferrin; and/or (vii) ethanolamine. In another embodiment, the composition can include (i) basal media; (ii) purified lipoprotein material; (iii) glutamine; (iv) albumin; (v) insulin; and/or (vi) transferrin. In a further embodiment, the composition can include (i) basal media; (ii) purified lipoprotein material; (iii) glutamine; (iv) albumin; (v) insulin; and/or (vi) transferrin; and/or (vii) peptone. In another embodiment, the composition can include (i) basal media; (ii) purified lipoprotein material; (iii) glutamine; (iv) albumin; (v) insulin; (vi) transferrin; and/or (vii) fetuin (such as Pedersen). In another embodiment, the composition can include (i) basal media; (ii) purified lipoprotein material; (iii) glutamine; (iv) albumin; (v) insulin; (vi) transferrin; and/or (vii) vitamin E. In a further embodiment, the composition can include (i) serum free media and/or (ii) purified lipoprotein material. In an additional embodiment, the composition can include (i) serum free media and/or (ii) purified lipoprotein material; and/or (iii) albumin.

In another aspect of the present invention compositions are provided that are useful as a cell culture medium that serves to increase the yield of biological products, such as proteins, produced by the cells cultured in the media. In a further aspect of the present invention, compositions are provided that are useful as a cell culture medium for a variety of cells. In one embodiment, the cell culture media of the present invention can be used for adherent cell culture. In another embodiment, the cell culture media described herein can be used for suspension cell culture.

In one embodiment, the method includes contacting hybridoma cells with a composition including: (i) basal media; (ii) purified lipoprotein material isolated as; (iii) glutamine; (iv) albumin; (v) insulin; (vi) transferrin; and/or (vii) ethanolamine, and/or maintaining the hybridoma cells under conditions suitable to support cultivation of the hybridoma cells in culture.

In another embodiment, the present invention is a method of cultivating cancer cells by contacting the cells with compositions that are useful as cell culture medium of the present invention and/or maintaining the cancer cells under conditions suitable to support cultivation of the cancer cells in culture. In a specific embodiment, the method includes contacting cancer cells with a composition including (i) basal media; (ii) purified lipoprotein material; (iii) glutamine; (iv) albumin; (v) insulin; and/or (vi) transferrin.

### DEFINITIONS

The terms “cell culture medium,” “culture medium” and “medium formulation” refer to a nutritive solution for culturing or growing cells.

A “serum-free” medium is a medium that contains no serum (e.g., fetal bovine serum (FBS), horse serum, goat serum, or any other animal-derived serum known to one skilled in the art).

The term “basal medium” refers to any medium which is capable of supporting growth of cells. The basal medium supplies standard inorganic salts, such as zinc, iron, magnesium, calcium and potassium, as well as trace elements, vitamins, an energy source, a buffer system, and essential amino acids. Suitable basal media include, but are not limited to Dulbecco's Modified Eagle's Medium (DMEM), DME/F12, Minimal Essential Medium (MEM), Basal Medium Eagle (BME), RPMI 1640, F-10, F-12, .alpha. Minimal Essential Medium (.alpha.MEM), Glasgow's Minimal Essential Medium (G-MEM), and Iscove's Modified Dulbecco's Medium.

The term “protein yield” refers to the amount of protein expressed by cultured cells, and can be measured, for example, in terms of grams of protein produced/ml medium. If the protein is not secreted by the cells, the protein can be isolated from the interior of the cells by methods known to those of ordinary skill in the art. If the protein is secreted by the cells, the protein can be isolated from the culture medium by methods known to those of ordinary skill in the art. The amount of protein expressed by the cell can readily be determined by those of ordinary skill in the art. The protein can be a recombinant protein.

The term “suspension culture” refers to cells in culture in which the majority or all of cells in culture are present in suspension, and the minority or none of the cells in the culture vessel are attached to the vessel surface or to another surface within the vessel (adherent cells). The “suspension culture” can have greater than about 50%, 60%, 65%, 75%, 85%, or 95% of the cells in suspension, not attached to a surface on or in the culture vessel.

The term “adherent culture” refers to cells in culture in which the majority or all of cells in culture are present attached to the vessel surface or to another surface within the vessel, and the minority or none of the cells in the culture vessel are in suspension. The “adherent culture” can have greater than 50%, 60%, 65%, 75%, 85%, or 95% of the cells adherent.

The term “purified lipoprotein material” refers to material (i) that can include any lipophilic compound that can be, for example, carried through the plasma by apolipoproteins, including but not limited to cholesteryl esters, unesterified cholesterol, triglycerides, fatty acids and/or phospholipids; and (ii) that is in a higher state of purity than that found naturally in biological materials such as tissue or brain homogenate. In preferred embodiments, the purified lipoprotein material constitutes up to 20, 30, 40, 50, 60, 70, 80 or 90 percent or higher by weight of the material being treated. In one embodiment, the lipoprotein and cholesterol are in substantially pure form, i.e., the material being treated consists essentially of lipoprotein material.

As used herein, the term “mammal” is meant to include any human or non-human mammal, including but not limited to porcine, ovine, bovine, rodents, ungulates, pigs, sheep, lambs, goats, cattle, deer, mules, horses, monkeys, dogs, cats, rats, and mice.

I. Cell Culture Media

The invention provides novel cell culture media compositions and methods to cultivate cells with the novel media. In general, cell culture media contains a base solution or “basal media” into which all of the desired components are added.

Basal media which can be used in the present invention include but are not limited to Iscove's Modified Dulbecco's Medium, RPMI 1640, Minimal Essential Medium-alpha. (MEM-alpha), Dulbecco's Modification of Eagle's Medium (DMEM), DME/F12, alpha MEM, Basal Medium Eagle with Earle's BSS , DMEM high Glucose, with L-Glutamine, DMEM high glucose, without L-Glutamine, DMEM low Glucose, without L-Glutamine, DMEM:F12 1:1, with L-Glutamine, GMEM (Glasgow's MEM), GMEM with L-glutamine, Grace's Complete Insect Medium, Grace's Insect Medium, without FBS, Ham's F-10, with L-Glutamine, Ham's F-12, with L-Glutamine, IMDM with HEPES and L-Glutamine, IMDM with HEPES and without L-Glutamine, IPL-41 Insect Medium, L-15 (Leibovitz)(2×), without L-Glutamine or Phenol Red, L- 15 (Leibovitz), without L-Glutamine, McCoy's 5A Modified Medium, Medium 199, MEM Eagle, without L-Glutamine or Phenol Red (2×), MEM Eagle-Earle's BSS, with L-glutamine, MEM Eagle-Earle's BSS, without L-Glutamine, MEM Eagle-Hanks BSS, without L-Glutamine, NCTC-109, with L-Glutamine, Richter's CM Medium, with L- Glutamine, RPMI 1640 with HEPES, L-Glutamine and/or Penicillin-Streptomycin, RPMI 1640, with L-Glutamine, RPMI 1640, without L-Glutamine, Schneider's Insect Medium or any other media known to one skilled in the art.

The compositions of the present invention can be used to culture a variety of cells. In one embodiment, the medium is used to culture eukaryotic cells such as plant and/or animal cells. The cells can be mammalian cells, fish cells, insect cells, amphibian cells or avian cells. The medium can be used to culture cells selected from the group consisting of MK2.7 cells, PER-C6 cells, CHO cells, HEK 293 cells, COS cells and Sp2/0 cells. MK2.7 (ATCC Catalogue Number CRL 1909) is an anti-murine VCAM IgG1 expressing Hybridoma cell line derived from the fusion of a rat splenocyte and a mouse Sp2/0 myeloma. MK2.7 is a non-adherent cell line that can be grown in serum-free media. Other types of cells can be selected from the group consisting of 5L8 hybridoma cells, Daudi cells, EL4 cells, HeLa cells, HL-60 cells, K562 cells, Jurkat cells, THP-1 cells, Sp2/0 cells; and/or the hybridoma cells listed in Table 2 or any other cell type disclosed herein or known to one skilled in the art.

Additional mammalian cell types can include, but are not limited to, including primary epithelial cells (e.g., keratinocytes, cervical epithelial cells, bronchial epithelial cells, tracheal epithelial cells, kidney epithelial cells and retinal epithelial cells) and established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK.sub.2 cells, Clone M-3 cells, 1-10 cells, RAG cells, TCMK-1 cells, Y-1 cells, LLC-PK.sub.1 cells, PK(15) cells, GH.₁ cells, GH₃ cells, L2 cells, LLC-RC 256 cells, MH.sub.1C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl.sub.1 cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM.sub.1C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, C_{II} cells, and Jensen cells, or derivatives thereof).

The medium disclosed herein can be used to culture cells in suspension or adherent cells. The compositions of the present invention are suitable for either adherent, monolayer or suspension culture, transfection, and cultivation of cells, and for expression of proteins or antibodies in cells in monolayer or suspension culture.

Cells supported by the medium of the present invention can be derived from any animal, such as a mouse or a human. The cells cultivated in the present media can be normal cells or abnormal cells (i.e., transformed cells, established cells, or cells derived from diseased tissue samples).

Cell culture can be performed using various culture devices, for example, a fermentor type tank culture device, an air lift type culture device, a culture flask type culture device, a spinner flask type culture device, a microcarrier type culture device, a fluidized bed type culture device, a hollow fiber type culture device, a roller bottle type culture device, a packed bed type culture device or any other suitable devise known to one skilled in the art.

In another aspect of the present invention compositions are provided that are useful as a cell culture medium that serves to increase the yield of biological products, such as proteins, produced by the cells cultured in the media. In one embodiment, compositions can increase the yield of biological products at least 25%, 30%, 50%, 100%, 200% or 300%. In another embodiment, the biological products produced can be a peptide, such as a therapeutic or diagnostic peptide, polypeptide, protein, monoclonal antibody, immunoglobulin, cytokine (such as interferon, for example, interferon alpha, beta or gamma), integrin, antigen, growth factor, cell cycle protein, hormone, neurotransmitter, receptor, fusion peptide, blood protein and/ or chimeric protein. The biological product can also be an IgG, IgM, IgE, IgA immunogliobulin, a signle chain antibody or fragment thereof, such as a sFv fragment, a linked antibody fragment, and/or a humanized antibody.

II. Compositions to Reduce the Use of Serum

Traditionally, serum has been used to supplement classical basal media to increase cell growth and protein yield. In a first embodiment, the composition can include (i) basal media; (ii) purified lipoprotein material; and (iii) a reduced concentration of serum, such as fetal bovine serum (FBS), relative to the use of serum alone to supplement basal media.

In embodiments of the present invention, the purified lipoprotein material can be derived from serum or plasma obtained from a mammal. In one embodiment, the purified lipoprotein material can be a cholesterol-rich fraction, optionally in association with low density lipoprotein (LDL) and/ or high density lipoprotein (HDL). In another embodiment, the purified lipoprotein material can be derived from bovine, horse, sheep, pig or human serum or plasma. In a further embodiment, the purified lipoprotein material can contain cholesterol, such as approximately 1, 2, 3, 4, 5, 6, 7, 8, 9, 10, 11, 12, 13, 14, 15, 16, 17, 18, 19, 20, 21, 22, 23, 24, 25, 30, 40 or 50 grams per liter, more particularly, 9, 10 or 11, grams per liter of cholesterol as determined, for example, by enzymatic assay; protein, such as approximately between 10 and 20 or 5 and 30 grams per liter, more particularly, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 grams per liter of protein, and/ or approximately between 0 and 10 or between 0 and 6 EU endotoxin per milligram of cholesterol, more particularly, 0, 0.5, 1, 1.5, 2, 2.5, 3, 3.5, 4, 4.5, 5, 5.5, 6, 7, 8, 9, 10, 15, 20, 25, or 30 EU endotoxin per milligram of cholesterol, such as determined, for example, by limulus amebocyte lysate. In another embodiment, the pH of the purified lipoprotein material can be between approximately 7 and 8, more particularly, between about 7.0 and 8.4, for example, about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.5, or 10. In other embodiments, the purified lipoprotein material does not contain detectable levels of contaminants or undesired materials, for example, immunoglobulin G (IgG), microbes, mycoplasm, and/or viral agents.

Traditionally, in cell culture, basal media is supplemented with about 10%, 15%, 20%, 25%, 30% or more serum, such as FBS. In this embodiment, basal media is supplemented with less than about 10%, 15%, 20%, 25%, or 30% serum, and more particularly, less than about 9.5, 9, 8.5, 8, 7.5, 7, 6.5, 6, 5.5, 5, 4.5, 4, 3.5, 3, 2.5, 2, 1.5, 1, 0.75, 0.5, or 0.25% serum, such as FBS, in combination with a purified lipoprotein material. In one embodiment, between 0.1 and 5%, 0 and 10% or 1 and 5% purified lipoprotein material can be used in the composition. In another embodiment, approximately 0.2, 0.3., 0.4, 0.5, 0.6., 0.7, 0.75, 0.8, 0.9, 1.0, 1.5, 2, 2.5, 3, 3.5, 4, 4.5, 5, 5.5, 6, 7, 8, 9, 10, 20 or 30% purified lipoprotein material can used. In further embodiments, 0.1 to 1.0%, 0.1 to 0.5, 0.5 to 1.0%, 0.5 to 1.5%, 0.1 to 2%, 1 to 2%, 1 to 5%, 5 to 10% or 10 to 20% purified lipoprotein material can be used.

III. Compositions to Replace Serum

The biochemical complexity of serum, such as FBS, can potentially complicate the downstream processing of the proteins of interests. The present invention also provides a unique formulation of complete media without the use of serum.

According to this aspect of the invention, the composition can include (i) basal media; (ii) purified lipoprotein material; (iii) insulin; iv) albumin; (v) sodium selenite; and (vi) transferrin.

The composition can include any amount of BSA that achieves the desired effect, including but not limited to approximately 2, 2.5, 3, 3.5, 4, 4.5, 5, 5.5, 6, 6.5, 7, 7.5, 8, 8.5 or 9 mg/ml albumin, such as BSA. In one embodiment, the composition can include 1 to 3, 1 to 5, 2 to 4, 2 to 7, 3 to 6, 5 to 9, 5 to 8, or 2 to 8 mg/ml albumin, such as BSA. The albumin can be bovine serum albumin (BSA) or human serum albumin (HSA). The albumin can be an “albumin substitute”, which can be any compound which may be used in place of bovine serum albumin (e.g., human serum albumin (BSA) or AlbuMAX.RTM.I) in the supplement of the invention to give substantially similar results as albumin. Albumin substitutes may be any protein or polypeptide source. Examples of such protein or polypeptide samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX.RTM.I, and AlbuMAX.RTM. II. In a specific embodiment, the composition can include 3.5 to 5.0 mg/ml, specifically, 4 mg/ml, BSA. The BSA can be a cell culture grade BSA, such as available from Serologicals, Inc.

The composition can also include any amount of transferrin that achieves the desired effect, including but not limited to approximately 3.0, 3.5, 4.0, 4.5, 5.0, 5.5, 6.0, 7.0, 10, 15 or 20 ug/ml transferrin. In one embodiment, the composition can include 2.5 to 3.0, 3.0 to 4.0, 3.0 to 5.0, 3.0 to 6.0, 4.0 to 8.0, or 6.0 to 10.0 ug/ml transferrin. In a specific embodiment, the composition can include 2 to 4 mg/ml, specifically 2.5 mg/ml, transferrin.

A transferrin substitute can also be used. A “transferrin substitute” refers to any compound which can replace transferrin and provides substantially similar results as transferrin. Examples of transferrin substitutes include but are not limited to any iron chelate compound, such as including, but not limited to, iron chelates of ethylenediaminetetraacetic acid (EDTA), ethylene glycol-bis(beta-aminoethyl ether)-N,N,N′,N′-tetraacetic acid (EGTA), deferoxamine mesylate, dimercaptopropanol, diethylenetriamine-pentaacetic acid (DPTA), and trans-1,2-diaminocyclohexane-N,N,N′,N′-tetraacetic adic (CDTA), as well as a ferric citrate chelate and a ferrous sulfate chelate. The transferrin can be iron saturated transferring, such as human transferrin. The transferring can be a cell culture grade transferrin, such as that available from Serologicals, Inc.

The composition can also include any amount of insulin that achieves the desired effect, including but not limited to approximately 5.5, 6.0, 6.5, 7.0, 7.5, 8.0, 8.5, 9.0, 9.5, 10, 10.5, 11, 11.5, 12, 15 or 20 ug/ml insulin. In one embodiment, the composition can include 5 to 7, 5.5 to 6, 7 to 10, 9 to 11, 8 to 12 or 10 to 15 ug/ml insulin.

An insulin substitute can also be used. The term “insulin substitute” refers to any zinc containing compound which may be used in place of insulin that provides substantially similar results as insulin. Examples of insulin substitutes include but are not limited to zinc chloride, zinc nitrate, zinc bromide, and zinc sulfate. Additional insulins are known to those of ordinary skill in the art, see, for example, Gilman, A. G. et al., Eds., The Pharmacological Basis of Therapeutics, Pergamon Press, New York, 1990, pp. 1463-1495. The insulin can be zinc insulin or human zinc insulin. The insulin can be cell culture grade insulin, such as available from Serologicals, Inc.

The composition can also include any amount of sodium selenite that achieves the desired result, including but not limited to approximately 1, 2, 3, 2.5, 3, 3.5, 4, 4.5, 5, 5.5, 6, 6.5, 6.7, 7.0, 7.5, 8.0, 9.0, 10, 15 or 20 ug/L sodium selenite. In one embodiment, the composition can include 1 to 5, 5.5 to 15, 6.0 to 7.0 or 6.0 to 10 ug/L sodium selenite.

The composition can include any amount of purified lipoprotein materialthat achieves the desired result, including but not limited to 0.2, 0.3., 0.4, 0.5, 0.6, 0.7, 0.75, 0.8, 0.9, 1.0, 1.5 or 2% purified lipoprotein materialin basal media. In one embodiment, 0.1 to 1.0%, 0.1 to 0.5, 0.5 to 1.0%, 0.5 to 1.5%, 0.1 to 2%, 1 to 2%, 1 to 5%, 5 to 10% or 10 to 20% purified lipoprotein materialcan be used in the composition.

In one specific embodiment, approximately 4 mg/ml BSA; approximately 5.5 ug/ml transferrin; approximately 10 ug/ml insulin; approximately 6.7 ug/L sodium selenite; and approximately 0.75% purified lipoprotein materialin basal media.

In another specific embodiment, the composition can include: approximately 3 to 5 mg/ml BSA, approximately 4.5 to 6.5 ug/ml transferrin, approximately 9 to 11 ug/ml insulin, approximately 6.0 to 7.0 ug/L sodium selenite and approximately 0.25 to 1.0% purified lipoprotein materialin basal media.

IV. Compositions to Boost the Performance of Serum-Free Media

Other types of serum-free edia have been developed to substitute for the use of serum in cell culture. Compositions of the present invention, such as those that contain a purified lipoprotein material, can be used as a supplemented to further boost the growth of cells and increase the yield of products produced

In one aspect, the composition can include (i) serum free media and (ii) purified lipoprotein material. In one embodiment, the serum free media is one of the media listed in Table 1. In another embodiment, the serum free media is either Hybridoma Media, animal component free or Ex-Cell (JRH Biosceinces, Inc.). In another embodiment, approximately 0.2, 0.3., 0.4, 0.5, 0.6, 0.7, 0.75, 0.8, 0.9, 1.0, 1.5 or 2% purified lipoprotein material is used. In a further embodiment, 0.1 to 1.0%, 0.1 to 0.5, 0.5 to 1.0%, 0.5 to 1.5%, 0.1 to 2%, 1 to 2%, 1 to 5%, 5 to 10% or 10 to 20% purified lipoprotein material can be used in the composition.

In another aspect, the composition can include (i) serum free media and (ii) purified lipoprotein material; and (iii) albumin. In one embodiment, the serum free media is one of the media listed in Table 1. In another embodiment, the serum free media is either Hybridoma Media, animal component free or Ex-Cell (JRH Biosceinces, Inc.). In a further embodiment, the composition can include approximately 0.1, 0.2, 0.3, 0.4, 0.5, 0.6, 0.7, 0.8, 0.9, 1, 2, 3, 4, 5, or 10% albumin, such as bovine serum albumin (BSA) or other types of albumin as described above. In another embodiment, approximately 0.2, 0.3., 0.4, 0.5, 0.6, 0.7, 0.75, 0.8, 0.9, 1.0, 1.5 or 2% purified lipoprotein material is used. In a further embodiment, 0.1 to 1.0%, 0.1 to 0.5, 0.5 to 1.0%, 0.5 to 1.5%, 0.1 to 2%, 1 to 2%, 1 to 5%, 5 to 10% or 10 to 20% purified lipoprotein material can be used in the composition.

In a specific embodiment, the composition can include ExCell, approximately 0.75% purified lipoprotein material and approximately 0.5% BSA. In another specific embodiment, the composition can include Hybridoma Medium, Animal Component-free, approximately 0.5% purified lipoprotein material and approximately 0.2% BSA.

V. Cell Culture Compositions and Methods to Enhance the Growth and Performance of Cells

The compositions and methods of the present invention can also be used to enhance the growth and product yield of hybridomas in cell culture.

In one aspect of the invention, the composition can include (i) basal media; (ii) purified lipoprotein; (iii) glutamine; (iv) albumin; (v) insulin; (vi) transferrin; (vii) ethanolamine. In one embodiment, the basal media can be DMEM. In one embodiment, between 0.1 and 5% purified lipoprotein material can be used in the composition. In another embodiment, approximately 0.1, 0.2, 0.3., 0.4, 0.5, 0.6., 0.7, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 3, 3.1, 3.2, 3.4, 3.5, 4, 5, 10, 15, 20, 25 or 30% purified lipoprotein material can be used. In further embodiments, 0.1 to 1.0%, 0.1 to 0.5, 0.5 to 1.0%, 0.5 to 1.5%, 0.1 to 2%, 1 to 2%, 1.5 to 2.0%, 2.0 to 2.5%, 1 to 5%, 5 to 10% or 10 to 20% purified lipoprotein material can be used. In a specific embodiment, the composition can include approximately 2.0% purified lipoprotein material.

The composition can include any amount of glutamine that achieves the desired effect, including but not limited to approximately 1, 2, 3, 4, 5, 6, 7, 8, 9, 10, 11, 12, 13, 14, 15, 20, 25 or 30 mM glutamine. In one embodiment, the composition can include 2 to 3, 3 to 4, 4 to 5, or 5 to 6 mM glutamine, particularly approximately 4 mM glutamine.

The composition can include any amount of albumin that achieves the desired effect, including but not limited to up to approximately 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 20, 25%; 3 to 5, 5 to 8% albumin. In one embodiment, the composition contains from approximately 0.2 to 0.4, 0.4 to 0.6, 0.6 to 0.9%, particularly approximately 0.5% albumin. In one embodiment, the albumin can be bovine serum albumin (BSA) or human serum albumin (HSA). In a specific embodiment, the composition can include 0.5% BSA. The albumin can be an “albumin substitute”, which can be any compound which may be used in place of bovine serum albumin (e.g., human serum albumin (BSA) or AlbuMAX.RTM.I) in the supplement of the invention to give substantially similar results as albumin. Albumin substitutes may be any protein or polypeptide source. Examples of such protein or polypeptide samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX.RTM.I, and AlbuMAX.RTM. II. The BSA can be a cell culture grade BSA, such as available from Serologicals, Inc.

The composition can also include any amount of insulin that achieves the desired effect, including but not limited to approximately 1, 2, 3, 4, 5, 6, 7, 8, 9, 9.5, 10, 10.5, 11, 11.5, 12, 13, 14, 15, 16, 17, 18, 19, 20, 25, 30, 40% or 16 to 18, 18 to 20 mg/L insulin. In one embodiment, the composition can include 7 to 9, 9 to 11, 11 to 13, specifically about or approximately 10 mg/L insulin. An insulin substitute can also be used. The term “insulin substitute” refers to any zinc containing compound which may be used in place of insulin that provides substantially similar results as insulin. Examples of insulin substitutes include but are not limited to zinc chloride, zinc nitrate, zinc bromide, and zinc sulfate. Additional insulins are known to those of ordinary skill in the art, see, for example, Gilman, A. G. et al., Eds., The Pharmacological Basis of Therapeutics, Pergamon Press, New York, 1990, pp. 1463-1495. In one embodiment, the insulin can be zinc insulin or human zinc insulin. The insulin can be cell culture grade insulin, such as available from Serologicals, Inc. In a specific embodiment, the insulin is human recombinant insulin such as that available from Serologicals.

The composition can also include any amount of transferrin that achieves the desired effect, including but not limited to approximately 0.1, 0.2, 0.3, 0.4, 0.5, 0.6, 0.7, 0.8, 0.9, 0.95, 1, 1.1, 1.2, 1.3, 1.4, 1.5, 1.6, 1.7, 1.8, 1.9, 2, 2.1, 2.2, 2.3, 2.4, 2.5, 3, 4, 5, 10, or 15 mg/L transferrin; 3 to 5, or 5 to 7 mg/L transferrin. In one embodiment, the composition can include 0.5 to 1.0, 1.0 to 1.5, 1.5 to 2.0 mg/L transferrin. In a specific embodiment, the composition can include approximately 1.0 mg/L transferrin. A transferrin substitute can also be used. A “transferrin substitute” refers to any compound which can replace transferrin and provides substantially similar results as transferrin. Examples of transferrin substitutes include but are not limited to any iron chelate compound, such as including, but not limited to, iron chelates of ethylenediaminetetraacetic acid (EDTA), ethylene glycol-bis(beta-aminoethyl ether)-N,N,N′,N′-tetraacetic acid (EGTA), deferoxamine mesylate, dimercaptopropanol, diethylenetriamine-pentaacetic acid (DPTA), and trans-1,2-diaminocyclohexane-N,N,N′,N′-tetraacetic adic (CDTA), as well as a ferric citrate chelate and a ferrous sulfate chelate. The transferrin can be iron saturated transferring, such as human transferrin. The transferring can be a cell culture grade transferrin, such as that available from Serologicals, Inc. In a specific embodiment, the transferring is human holo-transferrin, such as that available from Serologicals, Inc.

The composition can include any amount of ethanolamine that exerts the desired effect, including but not limited to approximately 1, 2, 3, 4, 5, 6, 7, 8, 8.5, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.5, 12, 13 to 15, or 15 to 20 μM ethanolamine. In one embodiment, the composition can include 9.0 to 9.5, 9.5 to 10, 10 to 10.5, or 10.5 to 11 μM ethanolamine. In a specific embodiment, the composition can include approximately 10 μM ethanolamine.

In other embodiments, the cell culture media can contain basal media, approximately between 1 and 4, 2 and 4, 1 and 3, 1 and 5, 0.5 and 5, 0.5 and 4.5, 3 and 5, 3.5 and 4.5 mM glutamine, approximately between 0.2 and 1.0%, 0.1 and 1.0%, 0.5 and 1.0%, 0.3 and 1.5%; 0.2 and 5%; 0.2 and 3% and 0.3 and 2% albumin; approximately between 1 and 10 mg/L, 8 and 12 mg/L, 5 and 15 mg/L or greater than 25 mg/L insulin; approximately between 0.5 and 9.5, 0.5 and 9.7, 0.5 and 1.5, 0.5 and 5, 0.5 and 7, and 0.5-9 mg/L transferrin; approximately 1 and 10 uM, 8 and 12 uM, 5 and 15 uM or greater than 25 uM ethanolamine, and approximately between 0.5% and 3%, 0.5% and 5%, 1% and 5%, 0.5% and 0.9%, 1.5% and 2.5%; and 1-3% of a purified lipoprotein material. In one specific embodiment, the composition of the present invention can include approximately 4 mM glutamine, approximately 2% purified lipoprotein material, approximately 0.5% BSA, approximately 10 mg/L insulin, approximately 1 mg/L transferrin and approximately 10 micromolar ethanolamine.

The present invention also includes a method of culturing cells using a involving contacting the cells with a composition described herein, including, but not limited to: (i) basal media; (ii) purified lipoprotein material; (iii) glutamine; (iv) albumin; (v) insulin; (vi) transferrin; and/ or (vii) ethanolamine, and maintaining the cells under conditions suitable to support cultivation of the cells in culture. In one embodiment of the present invention, the cells are eukaryotic cells, such as plant or animal cells or any other cell described herein. In a particular embodiment, the cells are MK2.7 cells, HEK 293 cells, CHO cells, PER-c6 cells, 5L8 cells, COS cells and Sp2/o cells.

In one embodiment, the preset invention provides a method of culturing hybridoma cells involving contacting the cells with a composition including (i) basal media; (ii) purified lipoprotein material; (iii) glutamine; (iv) albumin; (v) insulin; (vi) transferrin; and (vii) ethanolamine, and maintaining the cells under conditions suitable to support cultivation of the hybridoma cells in culture. In one embodiment, the hybridoma is one of the hybridomas listed in Table II. In a particular embodiment, the hybridoma is MK2.7.4. In a further embodiment, the hybridoma is 5L8.

In a specific embodiment, the preset invention is method of culturing hybridoma cells involving contacting the cells with a composition including (i) basal media; (ii) approximately 2% purified lipoprotein material; (iii) approximately 4 mM glutamine; (iv) approximately 0.5% BSA; (v) approximately 10 mg/L insulin; (vi) approximately 1 mg/L transferrin; and (vii) approximately 10 micromolar ethanolamine, and maintaining the cells under conditions suitable to support cultivation of the hybridoma cells in culture.

In another aspect of the invention, the composition can include (i) basal media; (ii) purified lipoprotein material; (iii) glutamine; (iv) albumin; (v) insulin; and (vi) transferrin. In one embodiment, the basal media is RPMI 1460. In one embodiment, between 0.01 and 5% purified lipoprotein material is used in the composition. In another embodiment, approximately 0.02, 0.03., 0.04, 0.05, 0.06., 0.07, 0.8, 0.09, 1, 1.5, 2, 2.5, or 3% purified lipoprotein material can be used. In further embodiments, 0.01 to 0.05 %, 0.05 to 0.1%, 0.1 to 0.15%, 0.2 to 0.3% purified lipoprotein material can be used. In other embodiments, any amount of purified lipoprotein material as disclosed herein can be used. In a specific embodiment, the composition can include approximately 0.1% purified lipoprotein material.

The composition can include any amount of glutamine that achieves the desired effect, including but not limited to approximately 1, 2, 3, 4, 5, 6, 7, 8, 9, or 10 mM glutamine. In one embodiment, the composition can include 2 to 3, 3 to 4, 4 to 5, 2 to 10, 1 to 20, 3 to 10, 3 to 6, or 5 to 6 mM glutamine, specifically approximately 4 mM glutamine.

The composition can include any amount of albumin that achieves the desired effect, including but not limited to up to approximately 0.2, 0.3, 0.4, 0.5, 0.6, 0.7, 0.8, 0.9, 1, 1.5, 2, 2.5, 3, 3.5, 3.5 to 5, 5 to 10, 10 to 20% albumin. In one embodiment, the composition contains from 0.5 to 1, 1 to 1.5, 1.5 to 2, specifically approximately 0.5% albumin. The albumin can be bovine serum albumin (BSA) or human serum albumin (HSA). In a specific embodiment, the composition can include 1.0% BSA. The albumin can be an “albumin substitute”, which can be any compound which may be used in place of bovine serum albumin (e.g., human serum albumin (BSA) or AlbuMAX.RTM.I) in the supplement of the invention to give substantially similar results as albumin. Albumin substitutes may be any protein or polypeptide source. Examples of such protein or polypeptide samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX.RTM.I, and AlbuMAX.RTM. II. The BSA can be a cell culture grade BSA, such as available from Serologicals, Inc.

The composition can also include any amount of insulin that achieves the desired effect, including but not limited to approximately 1, 2, 3, 4, 5, 6, 7, 8, 9, 9.5, 10, 10.5, 11, 11.5, 12, 13, 14, 15, 16 to 18, 18 to 20 mg/L insulin. In one embodiment, the composition can include 7 to 9, 9 to 11, 11 to 13, including approximately 10 mg/L insulin. An insulin substitute can also be used. The term “insulin substitute” refers to any zinc containing compound which may be used in place of insulin that provides substantially similar results as insulin. Examples of insulin substitutes include but are not limited to zinc chloride, zinc nitrate, zinc bromide, and zinc sulfate. Additional insulins are known to those of ordinary skill in the art, see, for example, Gilman, A. G. et al., Eds., The Pharmacological Basis of Therapeutics, Pergamon Press, New York, 1990, pp. 1463-1495. The insulin can be zinc insulin or human zinc insulin. The insulin can be cell culture grade insulin, such as available from Serologicals, Inc. In a specific embodiment, the insulin is human recombinant insulin such as that available from Serologicals.

The composition can also include any amount of transferrin that achieves the desired effect, including but not limited to approximately 0.1, 0.2, 0.3, 0.4, 0.5, 0.6, 0.7, 0.8, 0.9, 0.95, 1, 1.5, 2, 2.5, 3 to 5, or 5 to 7 mg/L transferrin. In one embodiment, the composition can include 0.5 to 1.0, 1.0 to 1.5, 1.5 to 2.0 mg/L transferring. In a specific embodiment, the composition can include approximately 1.0 mg/L transferrin. A transferrin substitute can also be used. A “transferrin substitute” refers to any compound which can replace transferrin and provides substantially similar results as transferrin. Examples of transferrin substitutes include but are not limited to any iron chelate compound, such as including, but not limited to, iron chelates of ethylenediaminetetraacetic acid (EDTA), ethylene glycol-bis(beta-aminoethyl ether)-N,N,N′,N′-tetraacetic acid (EGTA), deferoxamine mesylate, dimercaptopropanol, diethylenetriamine-pentaacetic acid (DPTA), and trans-1,2-diaminocyclohexane-N,N,N′,N′-tetraacetic adic (CDTA), as well as a ferric citrate chelate and a ferrous sulfate chelate. The transferrin can be iron saturated transferrin, such as human transferrin. The transferrin can be a cell culture grade transferrin, such as that available from Serologicals, Inc. In a specific embodiment, the transferring is human holo-transferrin, such as that available from Serologicals, Inc.

In one specific embodiment, the composition of the present invention can include approximately 4 mM glutamine, approximately 0.1% purified lipoprotein material, approximately 1% BSA, approximately 10 mg/L insulin, and approximately 1 mg/L transferrin.

In another embodiment, the composition can include (i) basal media; (ii) purified lipoprotein material; (iii) glutamine; (iv) albumin; (v) insulin; (vi) transferrin; and (vii) peptone. The composition can include any amount of peptone that exerts the desired effect, including but not limited to approximately 0.01, 0.02, 0.03, 0.04, 0.05, 0.06, 0.07, 0.08, 0.09, 0.1, 0.2, 0.3, 0.4, 0.5, 1, 2 or 3 % peptone. In one embodiment, the composition can include approximately 0.05 to 0.1, 0.1 to 0.2, 0.2 to 0.3, 0.3 to 0.5% peptone. In one specific embodiment, the composition of the present invention can include approximately 4 mM glutamine, approximately 0.1% purified lipoprotein material, approximately 1% BSA, approximately 10 mg/L insulin, approximately 1 mg/L transferrin, and approximately 0.5% peptone.

In further embodiment, the composition can include (i) basal media; (ii) purified lipoprotein material; (iii) glutamine; (iv) albumin; (v) insulin; (vi) transferrin; and (vii) fetuin. The composition can include any amount of fetuin that exerts the desired effect, including but not limited to approximately 2, 3, 4, 5, 6, 7, 8, 9, 10, 10.5, 11, 11.5, 12, 12.5, 13, 13.5, 14, 14.5, 15, 16 to 18, 18 to 20 micrograms/ml of fetuin. In one embodiment of the invention, fetuin is Pedersen's fetuin. In a particular embodiment, the composition contains approximately 8 to 10, 10 to 12, 12 to 14 micrograms/mL Pedersen's fetuin, specifically approximately 12.5 μg/mL Pedersen's fetuin, such as that available from Serologicals, Inc. In a specific embodiment of the present invention, the composition can include approximately 4 mM glutamine, approximately 0.1% purified lipoprotein material, approximately 1% BSA, approximately 10 mg/L insulin, approximately 1 mg/L transferrin, and approximately 12.5 μg/mL of Pedersen's fetuin.

In yet another embodiment, the composition can include (i) basal media; (ii) purified lipoprotein material; (iii) glutamine; (iv) albumin; (v) insulin; (vi) transferrin; and (vii) vitamin E. The composition can include any amount of vitamin E that exerts the desired effect, including but not limited to approximately 1, 2, 3, 4, 5, 6, 7, 8, 9, or 10 μM vitamin E. In one embodiment, the composition can include approximately 3 to 5, 5 to 8 or 8 to 10 μM vitamin E, specifically approximately 5 μM vitamin E. In a specific embodiment of the present invention, the composition can include approximately 4 mM glutamine, approximately 0.1% purified lipoprotein material, approximately 1% BSA, approximately 10 mg/L insulin, approximately 1 mg/L transferrin, and approximately 5 μM vitamin E

In a preferred embodiment, the composition of the present invention can include (i) basal media; (ii) purified lipoprotein material; (iii) glutamine; (iv) albumin; (v) insulin; (vi) transferrin; and (vii) peptone; (viii) fetuin; and (ix) vitamin E. In one specific embodiment, the composition can include approximately 4 mM glutamine, approximately 0.1% purified lipoprotein material, approximately 1% BSA, approximately 10 mg/L insulin, approximately 1 mg/L transferrin, approximately 0.1% peptone, approximately 12.5 micrograms/ml fetuin, and approximately 5 micromolar vitamin E.

The composition can be used to boost the growth and production of cells in culture, including eukaryotic cells. Thus, one aspect of the present invention is a method of cultivating cells comprising contacting the cells with a composition including: (i) basal media; (ii) purified lipoprotein material; (iii) glutamine; (iv) albumin; (v) insulin; (vi) transferrin.

A further aspect of the present invention is a method of cultivating cancer cells comprising contacting the cells with a composition of the present invention, including, but not limited to: (i) basal media; (ii) the purified lipoprotein material (iii) glutamine; (iv) albumin; (v) insulin; and/ or (vi) transferrin.

In one embodiment, the present invention provides a method of cultivating cancer cells comprising contacting the cells with a composition described herein, such as including: (i) basal media; (ii) purified lipoprotein material; (iii) glutamine; (iv) albumin; (v) insulin; (vi) transferrin; and/ or (vii) peptone.

In another embodiment, present invention is a method of cultivating cancer cells comprising contacting the cells with a composition including: (i) basal media; (ii) purified lipoprotein material; (iii) glutamine; (iv) albumin; (v) insulin; (vi) transferrin; and (vii) fetuin. In a specific embodiment, the fetuin is Pedersens fetuin.

In one embodiment, present invention is a method of cultivating cancer cells comprising contacting the cells with a composition including: (i) basal media; (ii) purified lipoprotein material; (iii) glutamine; (iv) albumin; (v) insulin; (vi) transferrin; and (vii) vitamin E.

In a further embodiment, the present invention is a method of cultivating cancer cells comprising contacting the cells with a composition including: (i) basal media; (ii) purified lipoprotein material; (iii) glutamine; (iv) albumin; (v) insulin; (vi) transferrin; (vii) peptone; and/ or (viii) fetuin.

In yet another embodiment, the present invention is a method of cultivating cancer cells comprising contacting the cells with a composition including: (i) basal media; (ii) purified lipoprotein material; (iii) glutamine; (iv) albumin; (v) insulin; (vi) transferrin; (vii) peptone; and/ or (viii) vitamin E.

In another embodiment, the present invention is a method of cultivating cancer cells comprising contacting the cells with a composition including: (i) basal media; (ii) purified lipoprotein material; (iii) glutamine; (iv) albumin; (v) insulin; (vi) transferrin; (vii) vitamin E; and/ or (viii) fetuin.

In a specific embodiment, the method of the present invention is a method of cultivating cancer cells comprising contacting the cells with a composition including: (i) basal media; (ii) purified lipoprotein material; (iii) glutamine; (iv) albumin; (v) insulin; (vi) transferrin; (vii) peptone; (viii) Pedersen's fetuin; and/ or (ix) vitamin E.

In a preferred embodiment, the method of the present invention is a method of cultivating cancer cells comprising contacting the cells with a composition including: (i) basal media; (ii) approximately 0.1% purified lipoprotein material; (iii) approximately 4 mM glutamine; (iv) approximately 1% BSA; (v) approximately 10 mg/L insulin; (vi) approximately 1 mg/: transferrin; (vii) approximately 0.1% peptone; (viii) approximately 12.5 μg/ml Pedersen's fetuin; and/ or (ix) approximately 5 μM vitamin E.

Any number of cancer cell lines are familiar to those skilled in the art. Representative examples of cancer cell lines that can be cultivated by the method of the present invention include but are not limited to the following cancer cell lines: human myeloma (e.g., KMM-1, KMS-11, KMS-12-PE, KMS-12-BM, KMS-18, KMS-20, KMS-21-PE, U266, RPMI8226); human breast cancer (e.g., KPL-1, KPL4, MDA-MB-231, MCF-7, KPL-3C, T47D, SkBr3, HS578T, MDA4355, Hs 606 (CRL-7368), Hs 605.T (CRL-7365) Hs 742.T (CRL-7482), BT474, HBL-100, HCC202, HCC1419, HCC1954, MCF7, MDA-361, MDA436, MDA453, SK-BR-3, ZR-75-30, UACC-732, UACC-812, UACC-893, UACC-3133, MX-1 and EFM-192A); ductal (breast) carcinoma (e.g., HS 57HT (HTB-126), HCC1008 (CRL-2320), HCC1954 (CRL-2338; HCC38 (CRL-2314), HCC1143 (CRL-2321), HCC1187 (CRL-2322), HCC1295 (CRL-2324), HCC1599 (CRL-2331), HCC1937 (CRL-2336), HCC2157 (CRL-2340), HCC2218 (CRL-2343), Hs574.T (CRL-7345), Hs 742.T (CRL-7482); skin cancer (e.g., COLO 829 (CRL-1974), TE 354.T (CRL-7762), Hs 925.T (CRL-7677)); human prostate cancer (e.g., MDA PCa 2a and MDA PCa 2b); bone cancer (e.g., Hs 919.T (CRL-7672), Hs 821.T (CRL-7554), Hs 820.T (CRL-7552), Hs 704.T (CRL-7444), Hs 707(A).T (CRL-7448), Hs 735.T (CRL-7471), Hs 860.T (CRL-7595), Hs 888.T.(CRL-7622); Hs 889.T (CRL-7626); Hs 890.T (CRL-7628), Hs 709.T (CRL-7453)); human lymphoma (e.g., K562); human cervical carcinoma (e.g., HeLA); lung carcinoma cell lines (e.g., H125, H522, H1299, NCI-H2126 (ATCC CCL-256), NCI-H1672 (ATCC CRL-5886), NCI-2171 (CRL-5929); NCI-H2195 (CRL05931); lung adenocarcinoma (e.g., NCI-H1395 (CRL-5856), NCI-H1437 (CRL-5872), NCI-H2009 (CRL-5911), NCI-H2122 (CRL-5985), NCI-H2087 (CRL-5922); metastatic lung cancer (e.g., bone) (e.g., NCI-H209 (HTB-172); colon carcinoma cell lines (e.g., LN235, DLD2, Colon A, LIM2537, LIM1215, LIM1863, LIM1899, LIM2405 LIM2412 , SK-CO1 (ATCC HTB-77), HT29 (ATCC HTB38), LoVo (ATCC CCL-229), SW1222 (ATCC HB-11028), and SW480 (ATCC CCL-228); ovarian cancer (e.g., OVCAR-3 (ATCC HTB-161) and SKOV-3 (ATCC HTB-77); mesothelioma (e.g., NCI-h2052 (CRL-5915); neuroendocrine carcinoma (e.g., HCI-H1770 (e.g., CRL-5893); gastric cancer (e.g., LIM1839); glioma (e.g., T98, U251, LN235); head and neck squamous cell carcinoma cell lines (e.g., SCC4, SCC9 and SCC25); medulloblastoma (e.g., Daoy, D283 Med and D341 Med); testicular non-seminoma (e.g., TERA1); prostate cancer (e.g., 178-2BMA, Du145, LNCaP, and PC-3). Other cancer cell lines are well known in the art.

### EXAMPLES

### Example 1

Method to Obtain Cholesterol-Rich Fraction From Bovine Serum

Starting material for a process according to the present invention can be maintained at a temperature of about 0° C. to about 50° C. Typically, the temperature is maintained at about 2° C. to about 15° C. A process according to the present invention can begin by subjecting the starting material to filtration. The filtration can be carried out utilizing one or more filtration steps. According to one embodiment, two filtration steps are sequentially utilized with filters having a nominal porosity of about 5 μ and about 1 μ. Any suitable filter in this range can be utilized.

If the starting material is serum, it is preferred to add a soluble salt, such as sodium citrate, to an ionic strength of about 0.25 to about 1. Other suitable salts include sodium chloride, sodium phosphate, potassium phosphate, ammonium sulfate and sodium sulfate. The addition of a soluble salt to the above concentration will increase the amount of cholesterol-rich fraction adsorbed in the subsequent silica adsorption step. Bovine or human plasma, for example, is normally collected by a method, which can include addition of citrate as an anti-coagulant. This salt concentration is usually sufficient for the adsorption step and no additional salt is needed. After adding the soluble salt, the solution can be mixed. Typically, the solution is mixed for about 30 minutes.

After addition of sodium citrate, other materials that can facilitate processing can be added to the starting material and any added soluble salt(s). According to one example, polyethyleneglycol (PEG) can be added to the filtered starting material. PEG having a range of molecular weights can be utilized. According to one example, PEG having an average molecular weight of about 3350 is utilized. However, PEG having greater or lesser molecular weights can also be utilized. Along these lines, PEG having an average molecular weight of about 6000 could be utilized. One of ordinary skill in the art, once aware of the disclosure contained herein would be able to determine the molecular weight of PEG to utilize with out undue experimentation. The PEG can be added in an amount of about 10 grams to about 15.6 grams for each liter of filtered starting material and sodium citrate, if utilized. After addition of the PEG the solution can be mixed. Typically, the solution is mixed for about 30 minutes, although shorter or longer mixing times can be utilized. While the addition of PEG can facilitate the purification process, it is not necessary.

After addition of the PEG, if utilized, the pH can be adjusted to a slightly acidic value. Along these lines, the pH can be adjusted to a value of about 5 to about 8. Typically, the pH is adjusted to a value of about 5.8 to about 6.2

After filtration, the lipoproteins in the filtered raw material are adsorbed onto an adsorbent. Any suitable adsorbent can be utilized. One example is silica-containing adsorbents. A silica adsorbent useful in this invention does not have a critical composition. Appropriate silica materials are the microfine silica available under the trademark Cabosil from Cabot Corporation and AEROSIL and SIPERNAT, such as the powdered silica SIPERNAT 50, manufactured by DeGussa and available from Cary Co. The silica is added to the liquid plasma or serum in an amount of about 1 to about 50 g/L, typically about 10 to about 20 g/L. The silica suspension in the liquid plasma or serum is then mixed for about 3 to about 4 hours.

The adsorption can be carried out at a slightly acidic pH. Along these lines, the adsorption can be carried out at a pH of about 5 to about 8. Typically, the adsorption is carried out at a pH of about 5.8 to about 6.2. According to one example, the adsorption is carried out at a pH of about 6. Additionally, the adsorption can be carried out at a temperature of about 15° C. to about 30° C. for about 2 hours to about 24 hours. After adding the adsorbent(s), the solution can be mixed. According to one embodiment, the solution is mixed for about 30 to about 6 hours.

After adsorption, the lipoprotein-adsorbent complex can be isolated and remaining portion of the raw material discarded. The isolation can be carried out as a simple phase separation utilizing a filter press.

Subsequent to isolating the lipoprotein-adsorbent complex, occluded serum proteins can be removed from the lipoprotein-adsorbent complex. The removal can be carried out utilizing a high salt buffer wash. According to one example, this can be accomplished by washing the lipoprotein-adsorbent complex with an aqueous salt solution containing about 0.15 M sodium chloride. Other useful salts can include sodium acetate and/or sodium phosphate. The pH of the solution can also vary. Typically, the pH of the wash solution is about 6.9 to about 7.1. Similarly, the temperature that the wash is carried out at can vary. Typically, the temperature is about 2° C. to about 30° C. The salt solution is used in an amount about 120 liters for about each kilogram of the lipoprotein-adsorbent complex. Typically, the total volume of wash solution utilized could be about 12,000 liters to about 24,000 liters. According to one embodiment, two wash steps are carried out, each utilizing about 12,000 liters of wash solution. According to another embodiment, two wash steps could be carried out, each utilizing about 6,000 liters of solution. However, the volume could be more or less. The washing can be accomplished as a batch process or in a continuous washing process. According to one embodiment, the washing procedure is carried out at least two times as a batch process to remove occluded proteins. According to one particular embodiment, a first wash is carried out utilizing about 12,000 liters of a solution that contains about 8.3 to about 9.2 grams sodium chloride per liter and about 2.1 to about 2.9 grams sodium phosphate per liter at a pH of about 6.9 to about 7.1 and at a temperature of about 2° C. to about 30° C. This embodiment also can include carrying out a second washing step with about 12,000 liters of a solution that can include about 2.1 to about 2.9 grams sodium phosphate per liter at a pH of about 6.9 to about 7.1 at a temperature of about 2° C. to about 30° C. In embodiments that utilize a filter press to carry out the washing, the washing, whether a batch or continuous process, continues until reaching a target absorbance for the wash collection. According to one embodiment, the absorbance is less than about 0.1 at 280 nm. After washing the isolated the lipoprotein-adsorbent complex, the material utilized to remove the occluded proteins can be discarded.

The purified lipoproteins can then be recovered from the adsorbent. The recovery can be carried out at an elevated pH. According to one embodiment, the recovery is carried out at a pH of about 10.5. According to another embodiment, the recovery is carried out by passing a high pH buffered solution through the lipoprotein-adsorbent complex until cholesterol is substantially removed from the adsorbent. After recovering the purified lipoproteins, the adsorbent is discarded.

A solution containing the recovered lipoproteins can then be filtered. The filtration can be carried out utilizing one or more filtration steps. According to one embodiment, two filtration steps are utilized. A first filtration step utilizes filters having a nominal porosity of about 1 μ. A second filtration step utilizes membrane filters having a porosity of about 0.45 μ. In this and any of the filtration steps described herein, other filters can be utilized having different porosities as long as the porosity results in filtering particles of the desired size. Those of ordinary skill in the art would be able to determine suitable filter porosities without undue experimentation.

After filtration, the recovered lipoproteins are exposed to an elevated pH. Exposing the recovered lipoproteins to the elevated pH appears to be significant in eliminating transmissible spongiform encephalopathy agent present in the recovered lipoproteins. Any suitable alkaline agent can be utilized to adjust the pH. According to one example, NaOH in a 1N solution was added to the recovered lipoproteins to achieve an elevated pH of between 10 to about 13. The exposure to the elevated pH can include any exposure from the briefest possible exposure up to many hours. Along these lines, the recovered lipoproteins can be exposed to an alkaline agent and the agent immediately neutralized. In such as case, the pH is not maintained at the elevated pH, but rather adjusted to the elevated value and then readjusted. The exposure in such a case can be as brief as practically possible. It appears, as discussed below, that even such a brief exposure can help to reduce TSE agent. Even though the pH exposure can be fleeting, the exposure is typically at least about 2, 3, 4, 5, 6, 7, 8, 9, 10, 11, or 12 hours. Typically, the pH is maintained at an elevated level for about 2 hours to about 12 hours. More typically, the pH is maintained at about 11 to about 13 for about 2 hours to about 8 hours. According to one embodiment, the solution is maintained at about pH of about 12 for about 8 hours. Longer periods of time can be utilized for the elevated pH exposure if deemed desirable and/or necessary. Those of ordinary skill in the art, once aware of the disclosure contained herein could determine acceptable pH levels and time periods without undue experimentation.

Time and pH appear to be related in that a lower pH can be utilized if the time at the lower pH is longer as compared to higher pH. For example, a pH of about 10.5 can be utilized for a time longer than about 8 hours. A solution maintained at a high pH can be maintained at the lower pH for a comparatively shorter period of time.

The elevated pH exposure step can be carried out at a temperature of about 18° C. to about 22° C. According to one particular embodiment, the elevated pH exposure step was carried out at a temperature of about 20° C. Temperature and time can also be related as pH and time. For example, a higher temperature can be utilized for a shorter period of time.

After the elevated pH exposure step, the recovered lipoproteins can be subjected to additional steps to isolate them. The maintenance steps can include concentration/diafiltration by ultrafiltration. In this portion of the process, the concentrated cholesterol-rich solution can be dialyzed against an alkaline and/or a pH neutral material to further remove adsorbent that can include silica. Examples of materials that could be utilized in the dialysis include sodium carbonate and water. In order to improve the effectiveness of this dialysis step, it is desirable for the cholesterol-rich solution to be at a pH of about 7 to about 13, typically at a pH of about 8. The pH can be adjusted to this value by alkaline or acidic addition. This can take place just prior to the dialysis step, but typically, for operating convenience, the pH is adjusted to this value before the cholesterol-rich solution is subjected to an ultrafiltration concentration step.

In the dialysis step, 8-12 volumes of deionized water can be utilized to remove the sodium carbonate. The resulting solution can then be concentrated by ultrafiltration prior to deionizing.

The concentration/defiltration by ultrafiltration can be carried out until the solution including the recovered lipoproteins is concentrated by about 15 percent to about 50 percent. Typically, the solution is concentrated by about 20 percent to about 25 percent.

The elevated pH exposure can be carried out at least partially during the concentration/diafiltration by ultrafiltration.

Next, the concentrated solution is filtered. The filtration can be carried out utilizing one or more filtration steps. The filters utilized to carry out the filtration can have the capability to remove particles in the range of about 0.1 μ to about 1.0 μ. According to one embodiment, the solution is filtered sequentially through filters having porosities of about 0.65 μ and about 0.2 μ.

Subsequent to filtration, the solution is subjected to a heat treatment. The heat treatment can include exposing the solution to elevated temperatures. The heat treatment can help to eliminate, reduce and/or inactivate viruses or prions that can be present in the solution.

The heat treatment typically can include exposing the solution to a temperature of at least about 60° C. for a period of time of about at least about 10 hours. Typically, the solution is exposed to a temperature of about 60° C. to about 80° C. for a period of time of about 10 hours to about 14 hours. The solution can be exposed to about the same elevated temperature continuously. Alternatively, the solution can be exposed to different temperatures during the heat treatment. According to one embodiment, the heat treatment is carried out in three stages including a first stage at a temperature of about 80° C. for a time period of about 1 hour, a second stage at a temperature of about 65° C. for a time period of about 3 hours, and a third stage at a temperature of about 60° C. for a time period of about 10 hours. Any suitable time and temperature can be utilized to result in the desired effects on the solution. According to one embodiment, the time and temperature utilized in the heat treatment are sufficient to eliminate, reduce and/or inactivate viruses, according to generally accepted techniques for virus elimination, reduction and/or inactivation.

After exposure to the heat treatment, the solution is subjected to filtration. The filtration can be carried out utilizing one or more filtration steps. The filters utilized to carry out the filtration can have the capability to remove particles in the range of about 0.1 μ to about 1.0 μ. According to one embodiment, four filtration steps are utilized to sequentially filter the solution with membrane filters of about 0.65 μ, about 0.45 μ, about 0.2 μ, and about 0.1 μ.

Next, final cholesterol and pH adjustments can be made. While it is not necessary in the process for production of the cholesterol-rich fraction, it is convenient that the product have a pH adjusted to about 7.0 to about 8.4 so that it is generally compatible with media employed for cell culture.

After adjustments to bring the cholesterol and pH to desired levels, the solution can be subjected to filtration. The filtration can be carried out utilizing one or more filtration steps. The filters utilized to carry out the filtration can have the capability to remove particles having a size in the range of about 0.1 μ to about 1.0 μ. According to one embodiment, four filtration steps are utilized to sequentially filter the solution with membrane filters of about 0.2 μ and about 0.1 μ. According to this embodiment, the solution is sequentially filtered through three filters having a porosity of about 0.1 μ. The solution typically is filtered into a sterile bulk container. Typically, the filtration is carried out in aseptic conditions.

The solution can then be filtered again. The filtering can be carried out as the final product is introduced into a container for the final product, in other words, a container that the product will be made available to customers in. Therefore, the filtering is typically carried out as point-of-fill filtration. The filtration can be carried out utilizing one or more filtration steps. The filters utilized to carry out the filtration can have the capability to remove particles in the range of about 0.2 μ to about 1.0 μ. According to one embodiment, two filtration steps are utilized to sequentially filter the solution with membrane filters of about 0.2 μ. According to this embodiment, the solution is filtered sequentially through two 0.2 μ filters. After the final filtration, the product is ready to package for shipment. The process as described above produces a final yield of about 80 to about 120 milliliters from about I liter of starting material serum.

This recovered purified lipoprotein/cholesterol complex is not pure cholesterol, but can be mixed with minor amounts of other materials, which passed through the production process. Along these lines, the complex typically is an aqueous mixture of cholesterol, phopholipids, and fatty acids. The resulting mixture has been found to be quite useful as a cell culture media supplement.

### Example 2

Use of EX-CYTE® to Reduce the Use of Serum

Methods

MK2.7 hybridoma cells were used. Seed inoculum was cultured in DME/F12 and FBS in spinners then adapted to less than 1% FBS by gradual reduction of FSB concentration. To begin the experiment, cells were washed in PBS and seeded at 1×10² cells/mL in each test condition. Batch cultures were sampled daily to monitor cell density and viability until culture viability was below 30%. Daily samples of culture supernatant were taken and processed to measure antibody production by ELISA.

Results

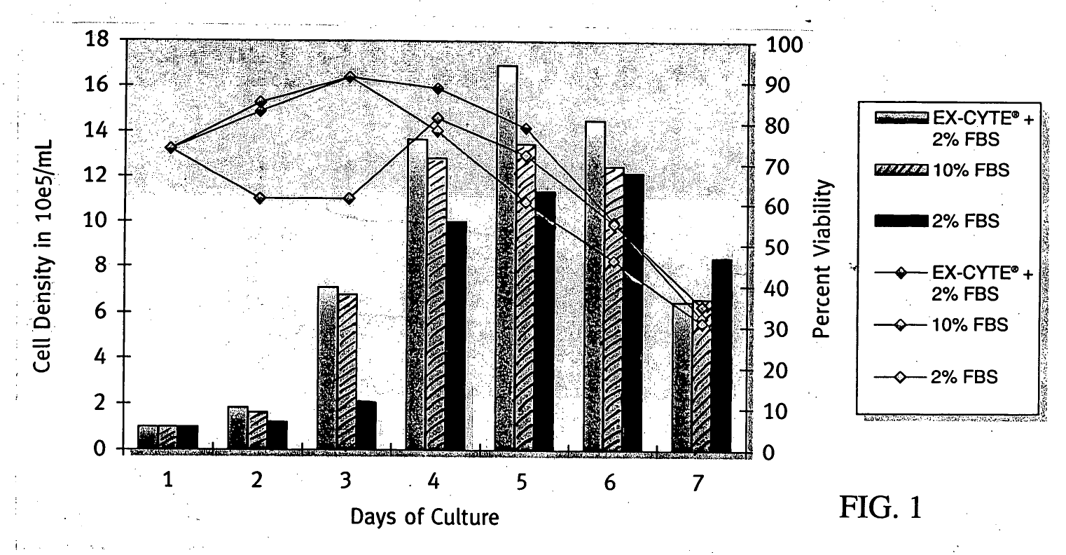

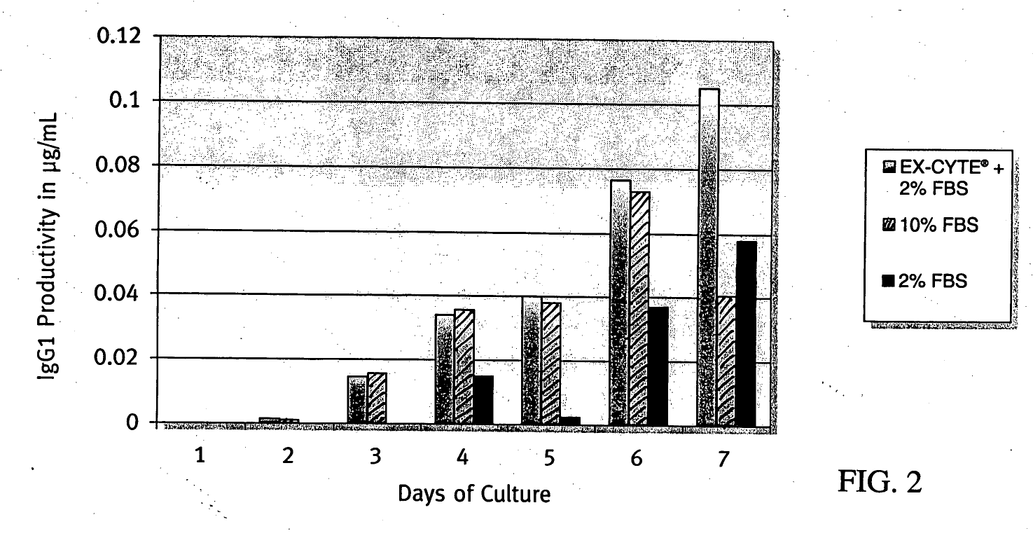

A combination of 0.5% EX-CYTE® and 2% FBS allowed for higher cell density and prolonged viability throughout the life of the culture as compared with 10% FBS (FIG. 1). The accumulative antibody level in the 0.5% EX-CYTE® and 2% FBS condition was more than double that of the 10% FBS culture on day 7 (FIG. 2). As a result, 0.5% EX-CYTE® effectively allowed the reduction of FBS from 10% to 2%.

### Example 3

Use of EX-CYTE® to Replace Serum

Methods

MK2.7 hybridoma cells were used. Seed inoculum was cultured in DMEM and FBS in spinners then adapted to less than 1% FBS by gradual reduction of FBS concentration. To begin the experiment, cells were washed in PBS and seeded at 1×10⁵ cells/mL in each media condition. The test condition consisted of 0.75% EX-CYTE® 0.4% BSA, 6.7 ug/L sodium selenite. 10 mg/L insulin and 5.5 mg/L transferrin. (BSA (Serologicals Catalogue Number 81-068). Insulin (Serologicals Catalogue Number 4506), Transferrin (Serologicals Catalogue Number 4465)). Batch cultures were sampled daily to monitor cell density and viability until culture viability was below 10%. Daily samples of culture supernatant were taken to measure antibody production by ELISA.

Results

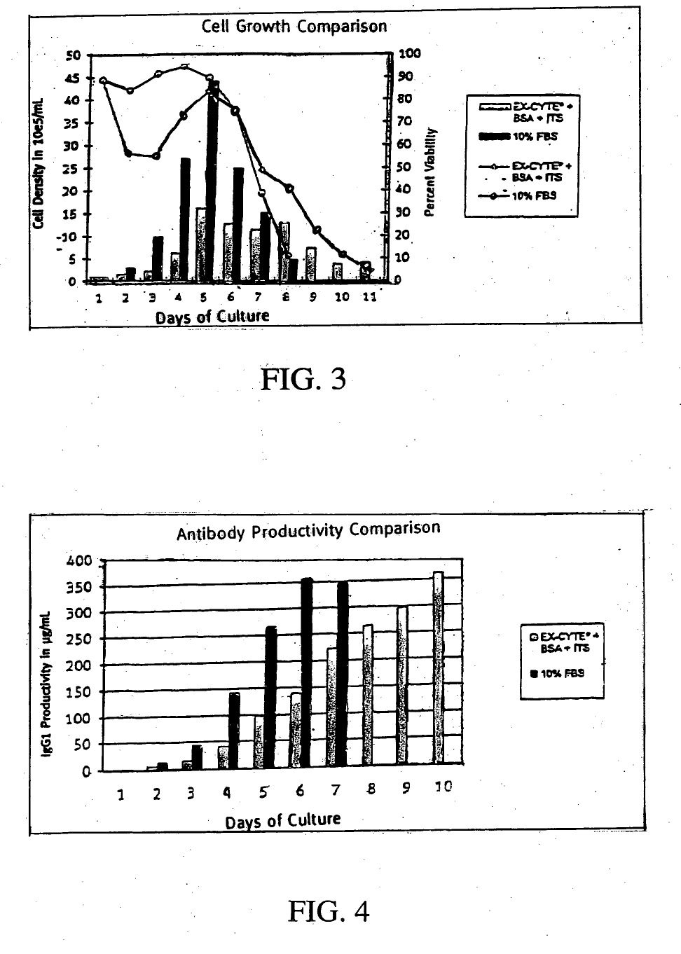

A combination of 0.75% EX-CYTE® and 0.4% BSA in DMEM constituted a complete serum-free media formulation. The temporary drop in culture viability in the test condition on days two and three was likely due to culture adaptation from low serum condition to serum-free condition (FIG. 3). FIG. 4 shows that comparable levels of production are achieved despite an overall drop in cell mass in the test condition, which is consistent with a shift in metabolic effort from growth in protein production. Productivity data of 10% FBS after day 8 was now shown because viability had decreased to less than 10%.

### Example 4

Use of EX-CYTE® to Boost Performance of Serum-Free Media

Methods

MK2.7 hybridoma cells were used. For each experiment, seed inoculum was adapted to each SFM according to the media manufacturers' recommendations. To begin the experiment, SFM adapted culture was seeded at 1×10⁵ cells/mL in each condition, Batch cultures were sampled daily to monitor cell density and viability until culture viability was below 10%. Daily samples of culture supernatant were taken and processed to measure antibody production by ELISA.

Results

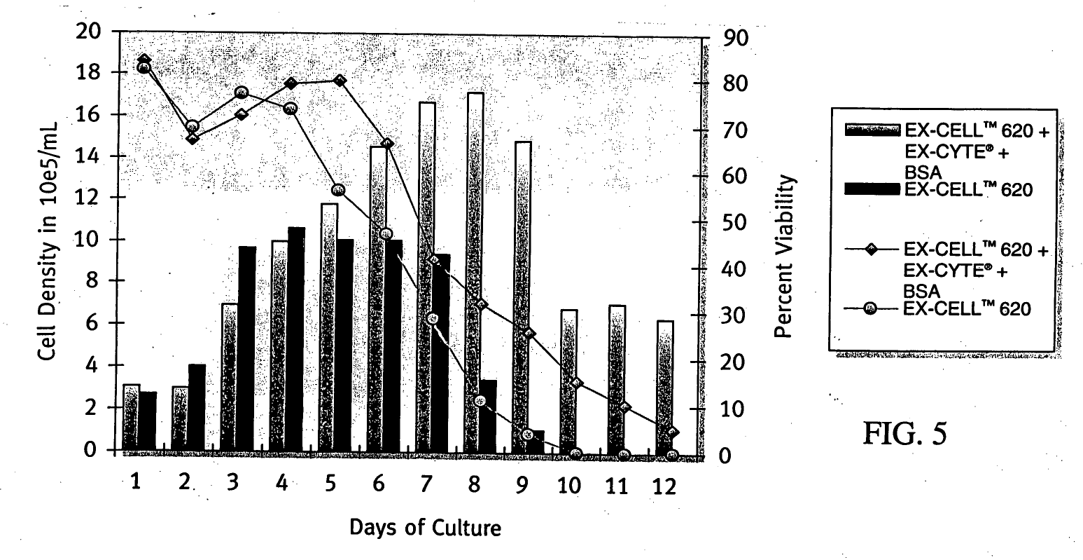

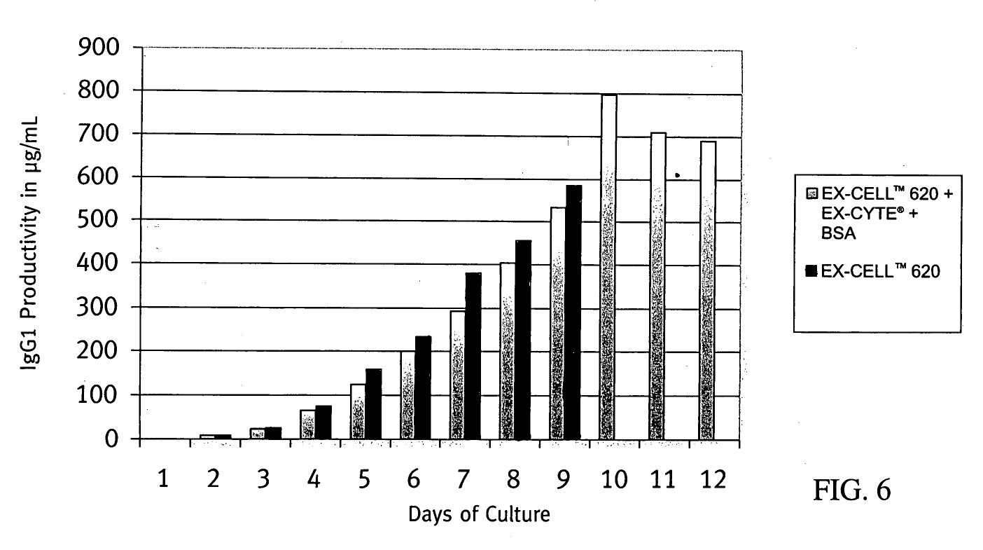

As shown in FIG. 5, the addition of 0.2% BSA and 0.5% EX-CYTE® to EX-CELL™ 620 Serum-free Media doubled the total cell mass over the life of the culture. The addition of EX-CYTE® and BSA significantly prolonged the life of the culture from eight days to greater than twelve days with increased viability. The peak IgG1 productivity in EX-CELL™ 620 supplemented with EX-CYTE® and BSA was 25% higher on day 10 compared with the EX-CELL™ 620 alone as shown in FIG. 6.

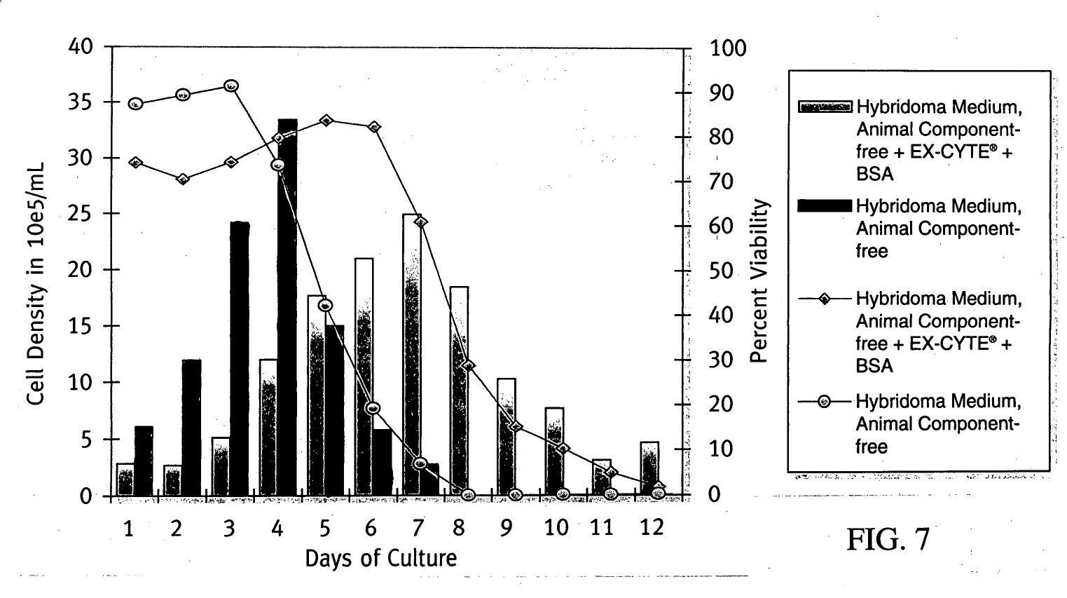

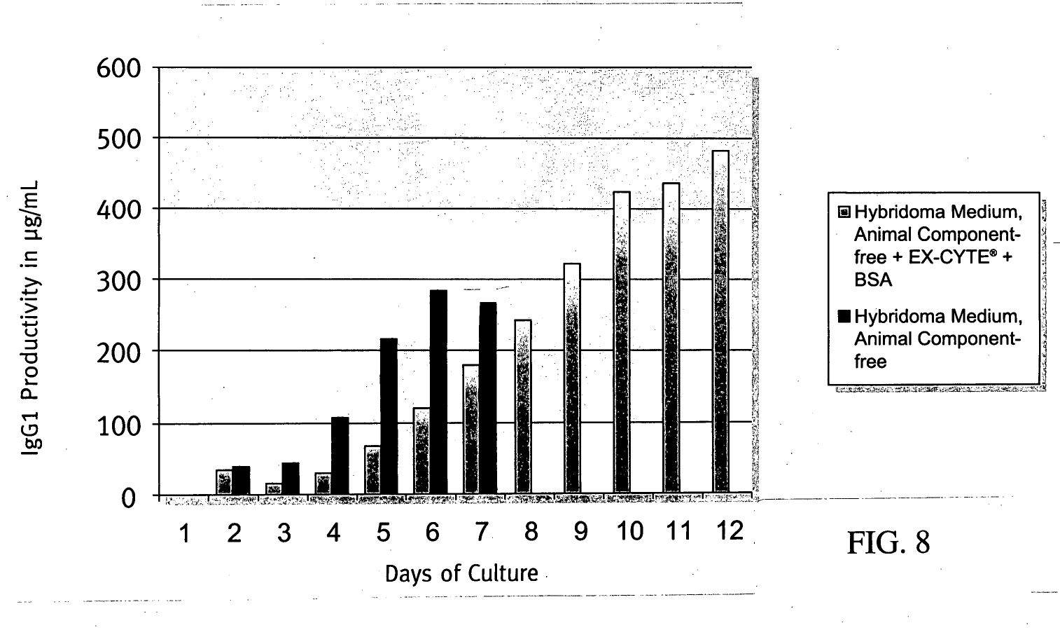

Similar effects were observed in Hybridoma Medium, Animal Component-free when supplemented with 0.5% EX-CYTE® AND 0.2% BSA. The life of the culture in Hybridoma Medium, Animal Component-free was prolonged from 7 days to greater than 12 days by the addition of EX-CYTE® and BSA (FIG. 7). The peak accumulated antibody level on day 12 in Hybridoma Medium, Animal Component-free was increased by 38% by the addition of EX-CYTE® and BSA (FIG. 8).

### Example 5

Use of XCF-1 Formulation to Boost Growth and Performance of Hybridoma Cell Lines

Methods

Six hybridoma cell lines were used, including MK2.7.4 hybridoma cells and 5C8 hybridoma cells. A composition termed XCF-1 was prepared containing DMEM (Gibco #11960-051), 4 mM glutamine (Gibco #25030-081), 2 % EX-CYTE ® (Serologicals Inc. # 81-129-081; Lot 420), 0.5% BSA (Serologicals Inc. #81-068; Lot 745), 10 mg/L human recombinant insulin (Serologicals Inc. #2002712), 1 mg/L human holo-transferrin (Serologicals, Inc. #4455-80), and 10 μM ethanolamine (Sigma # E-0135). Daily samples of culture supernatant were taken and processed to measure protein production by ELISA. Cell density was also measured.

Results

XFC-1 achieved cell density criteria in 5 out of 6 cell lines. XCF-1 also achieved protein production criteria in 4 of out of 6 cell lines. Data from these experiments in summarized below in Table 3. Data is normalized with 10% FBS representing 100%.

TABLE 3
XCF-1 Performance in Hybridoma Cells

| | Ave. Cell Density | Ave. Production |
|---|---|---|
| Hybridoma A | 1.15 | 0.923 |
| Hybridoma B | 1.28 | 0.9065 |
| Hybridoma C | 0.88 | 0.555 |
| Hybridoma D | 0.85 | 0.855 |
| 5C8.33 Hybridoma | 0.989 | 1.15 |
| MK2.7.4 | 0.979 | 0.986 |

Data for particular hybridoma cell lines is also given. As shown in Table 4, the addition of XCF-1 achieved cell density criteria and protein production criteria in MK2.7.4 cells.

TABLE 4
XCF-1 Performance in MK2.7.4 Cells

| | Cell Density | Protein Production |
|---|---|---|
| Experiment # 1 | 0.782 | 0.759 |
| Experiment # 2 | 1.22 | 1.24 |
| Experiment # 3 | 0.935 | 0.959 |
| Average: | 0.979 | 0.986 |
| Standard Deviation | +/−0.222 | +/−0.242 |

As shown in Figure Table 5, the addition of XCF-1 achieved cell density criteria and protein production criteria in 5C8 cells.

TABLE 5
XCF-1 Performance for 5C8 hybridoma cells

| | Cell Density | Protein Production |
|---|---|---|
| Experiment # 1 | 0.961 | 1.14 |
| Experiment # 2 | 1.078 | 1.47 |
| Experiment # 3 | 0.919 | 1.17 |
| Experiment # 4 | 0.999 | 0.80 |
| Average: | 0.989 | 1.15 |
| Standard Deviation | +/−0.068 | +/−0.272 |

### Example 6

Use of XCF-2 Formulation to Boost Growth and Performance of Cancer Cell Lines

Methods

Three cancer cell lines were used, including K562, Jurkat and EL-4. A composition termed XCF-2 was prepared containing RPMI 1640 (Sigma # R5886), 4 mM glutamine (Gibco #25030-081), 0.1% EX-CYTE® (Serologicals Inc. # 81-129-2; Lot 420), 1% (Serologicals Inc. # 81-068), 10 mg/L human recombinant insulin (Serologicals #2002712), 1 mg/L human holo-transferrin (Serologicals Inc. # 4455-80); 0.1% peptone (such as Primatone RL (Quest)), 12.5 micrograms/ml fetuin (Pedersen) (Serologicals Inc. # 4570-01), and 5 micromolar vitamin E (Sigma # T3251-5G). Cell density was measured.

Results

XCF-2 achieved cell density criteria in 3 out of 3 cell lines. Data from one cell line, 562, is summarized in Table 5. Data is normalized with 10% FBS representing 100%.

TABLE 6
XCF-2 Performance in K562 Cell Line

| | Cell Density |
|---|---|
| Experiment # 1 | 0.939 |
| Experiment # 2 | 0.796 |
| Experiment # 3 | 0.850 |
| Experiment # 4 | 0.903 |
| Experiment # 5 | 0.897 |
| Average | 0.877 |
| Standard Deviation | +/−0.055 |

Many modifications and other embodiments of the invention come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### Example 7

TABLE 7
Representative XCF2 Formulation

| XCF2 Constituents | Source | Catalog Number |
|---|---|---|
| EXCYTE (0.1%) | Serologicals | 81-129-2 |
| BSA (1%) | Serologicals | 81-068 (lot 745) |
| rHuman Insulin (10 mg/L) | Serologicals | 2002712 |
| Hu Holo-Transferin (1 mg/L) | Serologicals | 4455-80 |
| Pedersen Fetuin (12.5 mg/ml) | Serologicals | 4570-01 |
| Primatone RL (0.1%) or Soy | Quest | |
| Peptone | | |
| Sodium Selenite (25 nM) | Sigma | S-5261 |
| Vitamin E (5 mM) | Sigma | T3251-5G |

XCF2 Cell Lines

Numerous representative cell lines were been tested for growth with the XCF2 formulation in plate culture experiments. The following cell lines were used: K562—human hematopoietic, EL-4—mouse T-Lymphocyte, HL-60—human promyelocyte, Daudi—human B lymphoblast, HeLa—human cervical adenocarcinoma, THP-1—human monocyte and Jurkat—human T-Lymphocyte. All cell lines were adapted to 0.5% FBS, 1× Gibco ITS in RPMI with 4 mM Glutamine prior to testing. Some cell lines (Daudi and THP-1) were maintained and tested in XCF2 with the ATCC recommended additive of sodium pyruvate.

Experimental Design for Plate Studies

Cells were harvested from expansion growth in low serum adapted culture, washed and then plated at a density of 100,000 cells per well in each treatment. For all plate cultures, 24 well non-tissue culture treated plates were used. Cell viability and density were measured by propidium iodide incorporation and flow cytometry.

Surface Marker Expression Analysis by Flow Cytometry

K562 cells were used as a representative cell type for cancer/suspension cell lines. Investigations were designed to test expression of cell surface markers that have been identified as useful in research investigations. Two experimental designs were utilized to assess the expression of cell surface markers of b1-integrin (CD29), Fc receptor (CD32) and sialoglycoprotein (CD43) on K562 cells. In one design, expression after four days in culture in either low serum conditions (0.5% FBS), normal serum conditions (10% FBS) or XCF2 was measured. In the second experimental design, cells can be continually passaged in either XCF2 or 10% FBS and expression can be compared over a two week time period.

Results

EX-CYTE and BSA Optimization:

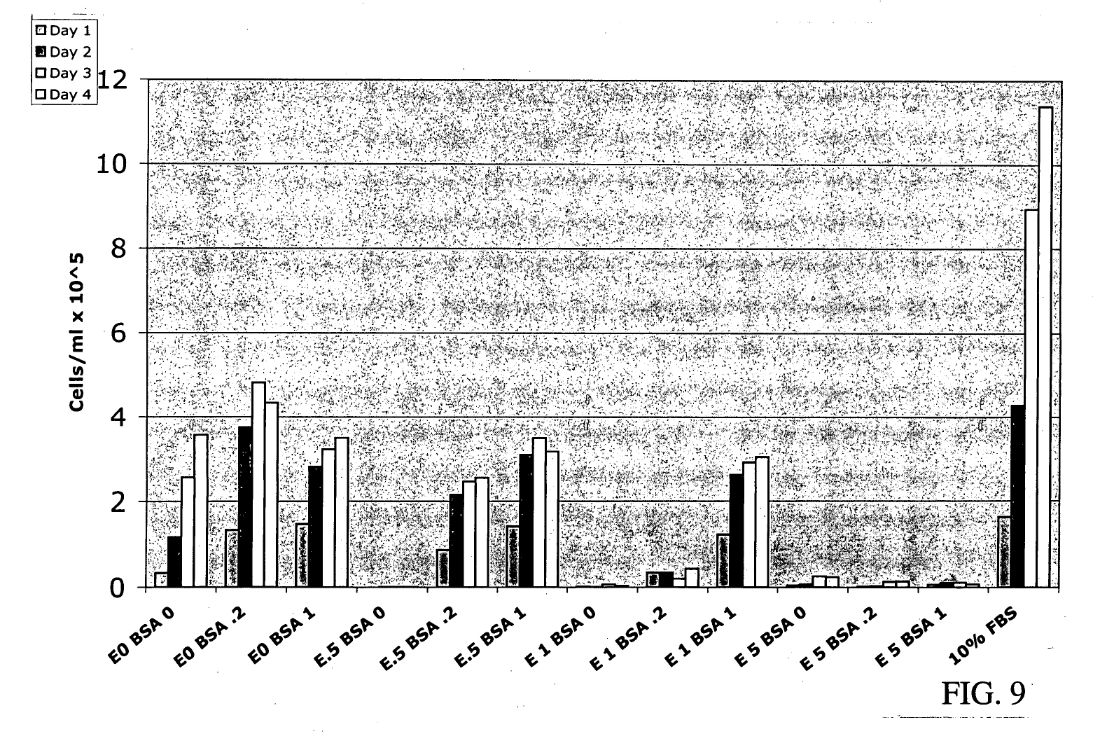

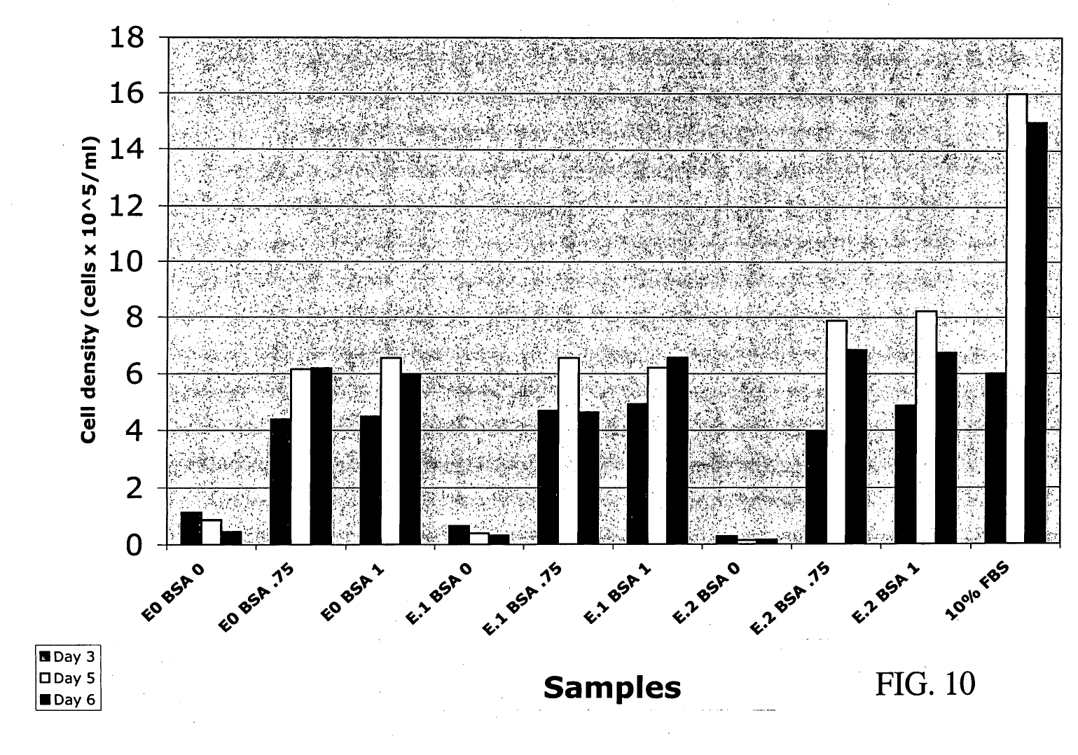

Initial studies were conducted with K562 cells in RPMI, Glutamine, and varying concentrations of EX-CYTE and BSA. FIG. 9 shows the gross range finding results and FIG. 10 demonstrates a refined range find that resulted in the current formulation of 0.1% EX-CYTE and 1% BSA to be selected.

Human Recombinant Insulin and Human Holo-Transferrin

Following the initial set of experiments, the concentration of Insulin and human holo-transferrin most beneficial was identified. From experiments conducted, 10 mg/L of Insulin and 1 mg/L of human holo-transferrin was determined to be an optimal amount.

Addition of Sodium Selenite and Ethanolamine

Sodium selenite was included at the level of 25 nM. Ethanolamine showed no effect and was excluded from this particular formulation.

Experimental Studies to Determine the Usefulness of Primatone, Fetuin and Vitamin E

Through a series of experiments including one to determine optimal concentrations and a second experiment investigating omission of these three components it was determined that Primatone at a concentration of 0.1%, Pedersen Fetuin at 12.5 mg/ml and Vitamin E at 5 mM would promote optimal cell density and growth. In the second set of experiments, omission studies showed that Primatone was a major contributing factor to growth promotion and viability. Pedersen Fetuin was important and contributed a promotion effect that boosted cell density approximately 10-20%.

Evaluation of XCF2 Performance When Primatone Rl is Substituted with Soy Peptone

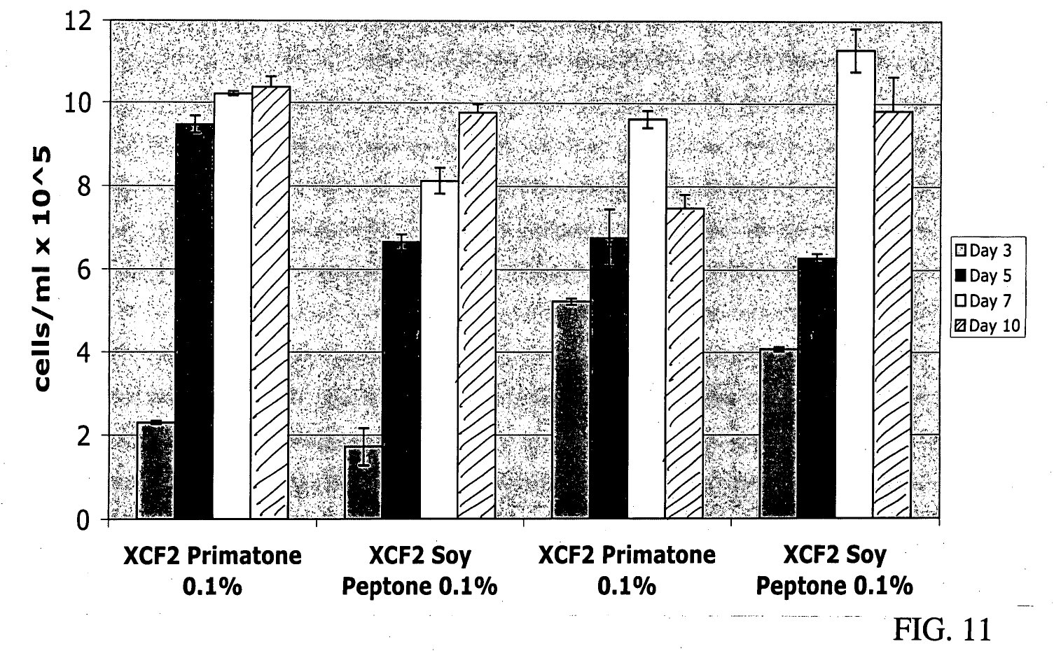

Soy Peptone can replace Primatone RL thus reducing the number of bovine derived constituents in the XCF2 formulation. In a growth promotion assay, K562 cells grown in XCF2 in RPMI containing either Primatone RL (0.1%) or Soy Peptone (0.1%) achieved equivalent cell density and viability. Performance from two replicate experiments are shown in FIG. 11.

Repeat Evaluations of XCF2 with K562 Cells

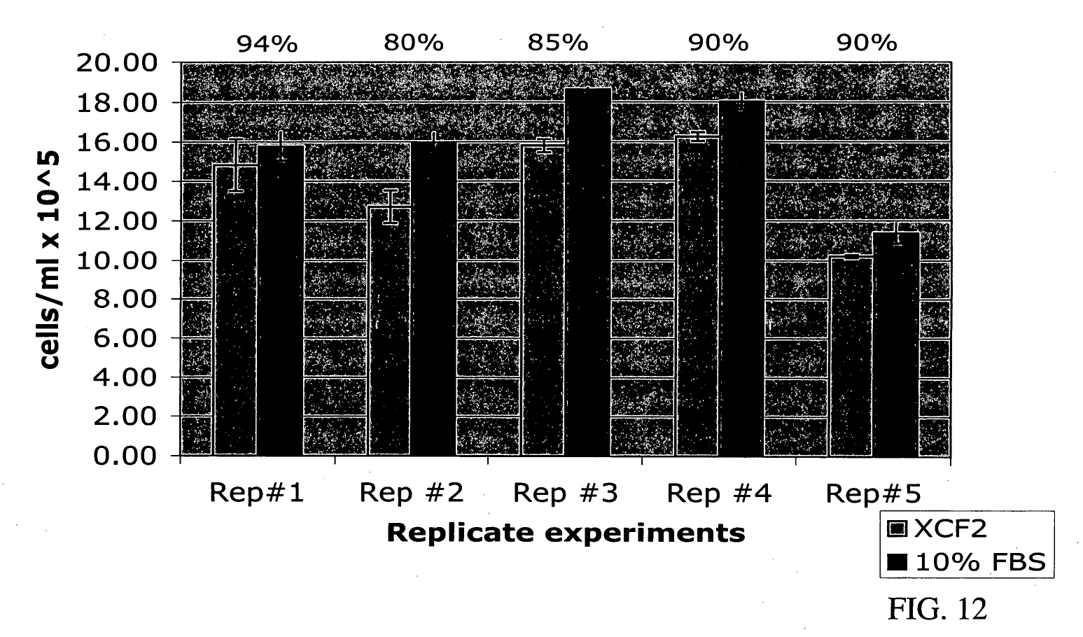

To ensure peak performance of the formulation, multiple replicates were tested to compare the final XCF2 formulation and the benchmark. In 4 of 5 experiments the criteria of 85% was met for cell density and viability as referenced to the 10% FBS benchmark. Cell density data for this set of experiments is shown in FIG. 12.

Evaluation of Xcf2 with Different Cell Lines

XCF2 has been tested in seven different cancer/suspension cell lines and the results from these tests have been presented here in tabular format. The cell lines include 6 human cell lines (K562, HL-60, Daudi, HeLa, THP-1 and Jurkat) and one mouse cell line (EL-4). Five of the seven cell lines matched the necessary performance criteria relative to the benchmark of 10% FBS. The minimal acceptable criteria was 0.85 for cell density and 0.85 for cell viability. Daudi, EL4, HeLa, HL-60 and K562 cells all performed at or above the minimal performance criteria. While Jurkat and THP-1 performance was above criteria for viability, it was below criteria for cell density. THP-1 cell density was 82% and Jurkat cell density was 73% of that achieved with 10% serum.

Cell morphology for six of the seven cell lines was as expected, with the one exception being HeLa cells. Although HeLa cell proliferation was approximately 2× greater in XCF2 than in 10% FBS, the cells were not adherent.

TABLE 8
Summary of results on XCF2 with different cancer cell lines

| Cell | Average Cell | Average Cell | Experimental | |
|---|---|---|---|---|
| Type | Density | Viability | Duration | #Replicates |
| Daudi | 0.91 | 0.95 | 7 Days | 2 |
| EL4 | 0.86 | 0.97 | 4 Days | 2 |
| HeLa | 2.02 | 1.22 | 10 Days | 2 |
| HL-60 | 1.26 | 1.10 | 14 Days | 1 |
| K562 | 0.88 | 0.95 | 7 Days | 5 |
| Jurkat | 0.73 | 0.96 | 7 Days | 2 |
| THP-1 | 0.82 | 0.97 | 7 Days | 2 |

XCF2 Performance in Basal Media

Two basal media (DMEM and RPMI) were selected for comparison of XCF2 performance. XCF2 performed well in both media.

K562 Growth in Continuous Culture and in Direct Adaptation from LN2 Storage into XCF2

Cells removed from LN2 storage and seeded directly into XCF2 achieved a doubling time of 20 hours within 10 days. The standard doubling time for K562 cells as reported in the literature is ˜20-21 hours. Using the K562 cells, the data demonstrates that the adaptability and doubling time for cells grown in XCF2 can achieve normal rates within the prescribed 14 day time-frame.

Surface Marker Expression on K562 Cells Grown in XCF2

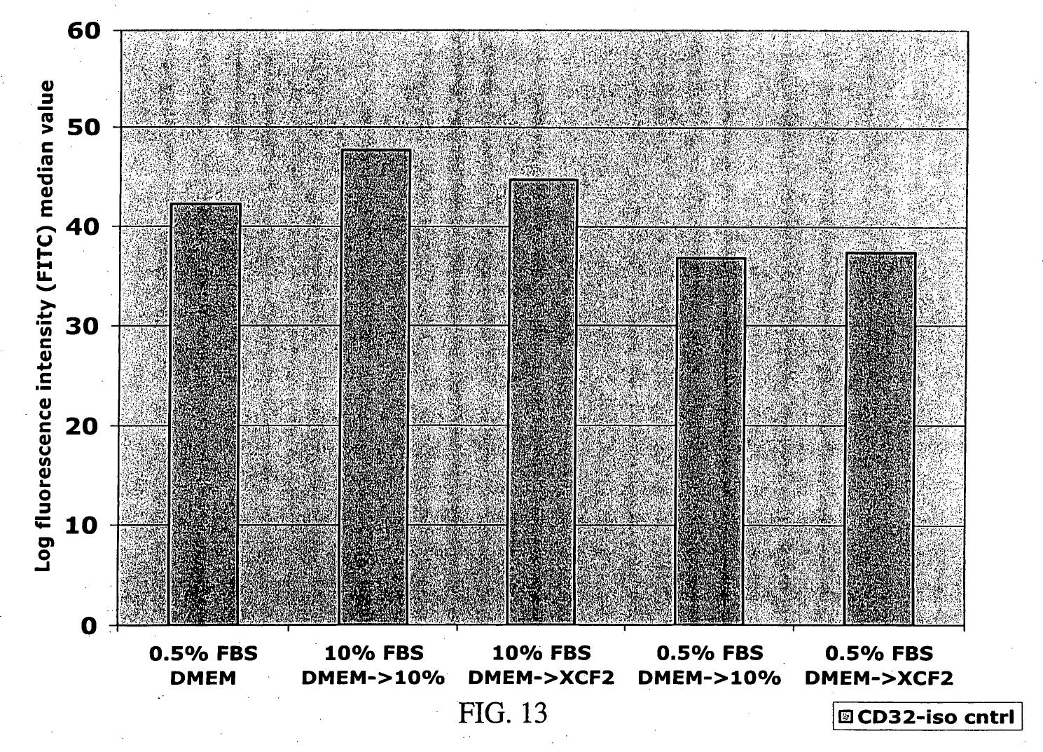

All three surface markers were expressed at comparable levels on K562 cells grown in XCF2 or 10% FBS in RPMI and DMEM. The graph in FIG. 13 shows the expression of CD32 minus the isotype control. Cells were grown in either 10% FBS or XCF2 for four days and then the surface marker expression was measured by flow cytometry.

TABLE 1
SERUM-FREE CULTURE MEDIA

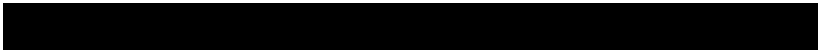

| | | | |
|---|---|---|---|
| PRODUCT | COMPOSITION | APPLICATIONS | CELLS |

ATHENA ENVIRONMENTAL SCIENCES
www.athenaes.com
UK suppliers Stratech at www.stratech.co.uk

| | | | | |
|---|---|---|---|---|
| 1 | BRFF-BMZERO™ | Complete serum-free medium | Designed for growing bitten | Human breast cells |
| | | | breast cells. Used for | |
| | | | establishing new epithelial cell | |
| | | | lines from explants of human | |
| | | | breast tissue and growing | |
| | | | immortalized normal breast | |
| | | | cell lines. | |
| 2 | BRFF-EPM2™ | Complete serum-free medium | Designed to grow human | Human epidermal cells |
| | | | epidermal-like cells | Human oesophageal epithelial cells |
| | | | Originally optimised for | Human skin explants |
| | | | human oesophageal epithelial | Human cancer cell lines |
| | | | cells. Explant cultures of | |
| | | | human skin in BRFF-EPM2 | |
| | | | yield outgrowths of epithelial | |
| | | | cells. Also useful for culturing | |
| | | | some human cancer cell lines. | |
| 3 | BRFF-HPC1™ | Complete serum-free medium. | Designed for establishing new | Human prostate tissue |
| | | Contains dihydrotesterone. | cell lines from human prostate | |
| | | | tissue. Epithelial cell lines, | |
| | | | from both benign prostatic | |
| | | | hyperplasia and prostatic | |
| | | | carcinoma have been | |
| | | | established and maintained in | |
| | | | this medium. | |
| 4 | BRFF-P4-8F™ | Complete serum-free medium. | Designed to grow | Normal prostatic cell line 267-B1 |
| | | | immortalized normal prostatic | Prostatic cancer cell lines, e.g. |
| | | | cell line 267-B1. Also supports | PC-3 |
| | | | the growth of certain | |
| | | | established human prostatic | |
| | | | cancer cell lines such as PC-3. | |
| 5 | Serum-free Media Screening Kit | Kit contains a 100 ml sample of | Screening kit is intended for | |
| | | each of five serum-free media, | researchers seeking to identify | |
| | | ready for use. These are BRFF- | the most appropriate serum- | |
| | | BMZERO™, BRFF-EMP2™, | free medium for a specific cell | |
| | | BRFF-P4-8F™, DMEM/F12, | type. | |
| | | and IMDM. | | |

ATLANTA BIOLOGICALS
www.atlantabio.com

| | | | | |
|---|---|---|---|---|
| 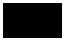 | 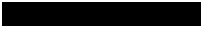 | 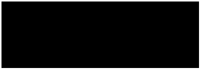 | 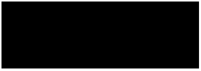 | 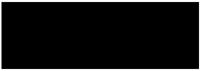 |
| 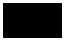 | 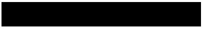 | 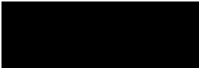 | 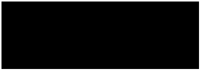 | 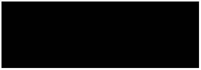 |

BD BIOSCIENCES/BECTON, DICKINSON & COMPANY
www.bdbiosciences.com

| | | | | |
|---|---|---|---|---|
| 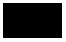 | 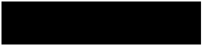 | 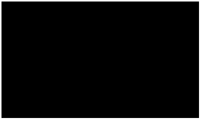 | 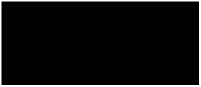 | 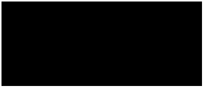 |
| 9 | BD Cell™ MAb Media, Serum | Complete serum-free HEPES | Supports a wide variety of | Various myeloma fusion partners |
| | Free | based medium. Contains | myeloma fusion partners and | and hybridomas including Sp2/0, |
| | | bovine serum albumin, L- | hybridomas. Designed to | NS-1, P3X63Ag9, and FOX-NY. |
| | | Glutamine and phenol red. | enhance monoclonal antibody | as well as secreting cell lines |
| | | Without pluronic acid or other | production. | such as CHO. |
| | | surfactants. | | |
| 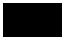 | 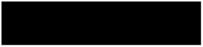 | 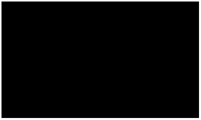 | 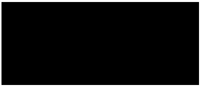 | 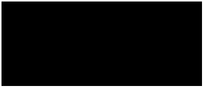 |
| 11 | BD Hepato-STIM™ | Serum-free, fully defined | | Hepatocytes |
| | | culture medium. | | |

BIOCHROM
www.biochrom.de
Products also supplied by Autogen-Bioclear www.autogen-bioclear.com

| | | | | |
|---|---|---|---|---|
| 12 | HybridomaMed DIF 1000 | Serum-free medium based on a | Developed for growth of | Hybridoma |
| | | 1:1 mixture of Iscove's | hybridoma, but also suitable | YAC-1 (mouse T-cell Lymphoma) |
| | | medium and Ham's P12, | for other cell lines. | HeLa (human epitheloid cervical |
| | | supplemented with transferrin, | | carcinoma) BJA-B (human EBV- |
| | | insulin and a BSA/oleic acid | | negative carcinoma) |
| | | complex. | | BHK-21 (Syrian hamster kidney) |
| | | | | L-psv 129 mouse L-fibroblast |
| 13 | MCDB 153 | Serum-free basal medium, | Supports the growth of human | Keralinocytes |
| | | requires supplementation with | keratinocytes. | |
| | | EGF, insulin, hydrocortisone, | | |
| | | ethanolamine and | | |
| | | phosphethanolamine. | | |
| 14 | MCDB 153 complete medium | Serum-free ready to use | Supports the growth of human | Keratinocytes |
| | | medium | keratinocytes. | |
| 15 | Octomed | Serum-free and protein-free | Designed for growth of | CHO cells |
| | | medium. | Chinese Hamster Ovary cells. | |
| 16 | PFEK-1 | Serum-free and protein-free | For proliferation of VERO | VERO cells |
| | | medium. | cells, for propagation of | |
| | | | human pathogenic viruses | |
| | | | (Coxsackie B4, herpes simplex | |
| | | | type 1 and 2, measles, polio | |
| | | | type 1-3). | |
| 17 | Sebomed Complete medium | Serum-free. Modified | Optimised for growth of | Human sebaceous gland cell line |
| | | DMEM/Ham's F-12 (1:1) | human sebaceous gland cell | SZ9S |
| | | Supplemented with EGF rh, | line SZ95 in vitro | |
| | | BPE, BSA, linoleic acid. | | |
| | | Formulation available on | | |
| | | request. | | |
| 18 | TNB 100 Medium | Serum-free medium | Optimised for cultivation of | Neuronal cell lines (mouse, chick |
| | | | neuroblastoma x glioma cells. | and rat). Human cytotoxic T-cells. |
| | | | Also useful for study of non- | |
| | | | neuronal cells such as human | |
| | | | cytotoxic T-cells. | |

BIOLOGICAL INDUSTRIES
www.bioind.com

| | | | | |
|---|---|---|---|---|
| 19 | BIOTARGET-1 | Serum-free medium | For use with mononuclear | Mononuclear cells (lymphocytes |
| | | | cells from peripheral blood. | and monocytes) |
| | | | Applications include | |
| | | | activation of mononuclear | |
| | | | cells with mitogens; activation | |
| | | | with lymphoid cells; | |
| | | | production of IL-2 and IL-3; | |
| | | | generation of LAX and TIL; | |
| | | | generation of natural killer | |
| | | | cells and cytotoxic T cells. | |
| | | | activation of macrophages; | |
| | | | proliferation of HIV, | |
| | | | retroviruses in T cells for | |
| | | | vaccine development. | |
| 20 | BIO-MPM-1 | Serum-free media. Contains | Multi-purpose media for | Various |
| | | insulin | adherent cells | |
| | | Without albumin. growth | | |
| | | factors, or hormones (other | | |
| | | than insulin) | | |
| 21 | BIOCHO-1 SFM Base | Serum-free base media, | BIOCHO-1 for adherent CHO | CHO cells of various kinds |
| | & BIOCHO-2 SFM Base | contains amino acids, vitamins. | cells and BIOCHO-2 for | |
| | BIOGRO-CHO SFM | salts, lipids, trace elements. | suspension cultures. | |
| | Supplement | Supplement for above base. | | |
| | | containing proteins. | | |
| | | The complete medium does not | | |
| | | contain albumin, growth | | |
| | | factors, or hormones (other | | |
| | | than insulin) | | |
| 22 | BIOINSECT-1 | Serum-free medium | Culture of lepidopteran insect | SF-9 |
| | | | cells | |
| 23 | Serum-free Cell Freezing | Serum-free medium for | Cryopreservation medium | 3T3, BGM, Vero, Hep2, BSC1 |
| | Medium | freezing cells. Contains | | |
| | | methylcellulose and DMSO | | |

BIO MEDIA
www.biomediaworld.com

| | | | | |
|---|---|---|---|---|
| 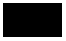 | 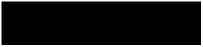 | 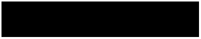 | 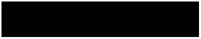 | 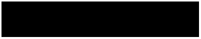 |

CAMBREX
www.cambrex.net

| | | | | |
|---|---|---|---|---|
| 25 | BioPro 1 | Low protein, serum-free liquid | Supports CHO cell lines | CHO |
| | | medium, without L-glutamine | | |
| | | and without glucose. | | |
| 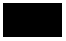 | 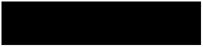 | 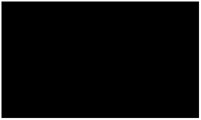 | 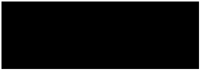 | 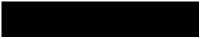 |
| 27 | UltraCHO™ | Serum-free modified DMEM: | Optimised to support the | CHO lines |
| | | F12 base, supplemented with | growth of transfected and non- | HeLa cells (suspension or attached) |
| | | insulin, transferring, and | transfected CHO cells | Human leukaemia cell lines |
| | | proprietary purified proteins. | | |
| | | Contains L-glutamine. | | |
| 28 | PC-1™ | Complete liquid media system | All purpose medium for | Cell lines: HeLa, MRC-5, |
| | | with frozen supplement. Low | culture of primary cells and | BHK-21, WI-38, NRK, 3T3, |
| | | protein and serum-free, | anchorage dependent cell lines | CHO-K1, HTB-72, HRB-4, WISH, |
| | | Modified DMEM/F12 base, | | VERO, MDCK, STO, Hep-2, |
| | | contains HEPES buffering | | SIRC, C6, T9, ARL6T. |
| | | system, insulin, transferrin and | | Primary cells: |
| | | fatty acids. | | Human neuroblastoma, foreskin |
| | | | | fibroblast, bladder carcinoma, renal |
| | | | | papillary collecting tubule, colon |
| | | | | epithelium, colon carcinoma. Rat |
| | | | | dermal fibroblast, mammary |
| | | | | carcinoma, neonatal normal cardiac |
| | | | | muscle, astrocytes, thyroid |
| | | | | epithelium. Baboon spinal ganglia, |
| | | | | swine testes cell, bovine kidney. |
| 29 | UltraCULTURE™ | Complete serum-free media | All purpose medium to | Human cells: HEL, N-10, HeLa, |
| | | consisting of a DMEM: F12 | support he growth of a wide | HuL-1,2, HuK-1, HuS-1 AT, HEC, |
| | | base, supplemented with | variety of both adherent and | HuL-1,2, K-562, HNK, HTC29, |
| | | bovine insulin, bovine | non-adherent cell lines. Has | TT, MB231, U138. |
| | | transferrin and purified mixture | been used to grow cells of | Mouse cells: FM3A, NS-1, L, |
| | | of bovine serum proteins | primary origin and established | P388D1, P815, T3, B32. |
| | | including albumen | cell lines. Used to grow cells | Rat cells: RPL-1, RSP-2, RLG-1, |
| | | | of lymphoid origin, including | Lynn-1, RCR-1, 235-1, MMQ, GC |
| | | | monocyte and macrophage cell | GH3, CA77, Ras-1. |
| | | | lines; epithelial and | Monkey cells: JTC-12, COS1, and |
| | | | fibroblastic cells; fusion of | CO57. |
| | | | cells during hybridoma | |
| | | | formation, and generation of | |
| | | | viral particles for use in | |
| | | | vaccine production. | |
| 30 | CellGro® SCGM | Serum-free medium containing | To sustain the growth of | Human hematopoietic progenitor |
| | | only human derived or | human hematopoietic | cells |
| | | recombinant human proteins | peripheral blood progenitor | |
| | | Without cytokines. or other | cells for gene therapy research. | |
| | | growth factors. | | |
| 31 | UltraDOMA™ | Serum-free medium composed | Supports the growth of | Murine hybridomas |
| | | of RPMI-1640 base, | murine, human and chirneric | NS-1 derived myelomas |
| | | supplemented with bovine | hybridomas for monoclonal | SP-2 derived myelomas |
| | | insulin, bovine transferring and | antibody production in batch | |
| | | bovine albumen, | culture and in hollow fibre | |
| | | Without L-glutamine. | bioreactors. | |
| 32 | UltraDOMA-PF™ | Serum-free and protein-free | Supports the growth of | Murine hybridomas |
| | | medium. Contains L-glutamine | murine, human and chimeric | NS-1 derived myclomas |
| | | | hybridomas for monoclonal | SP-2 derived myclomas |
| | | | antibody production. Designed | Rat hybridoma |
| | | | for lab or industrial scale use. | Some transfected CHO cell lines |
| | | | | Human lymphoid origin cells |
| | | | | Murine lymphoid origin cells |
| 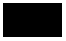 | 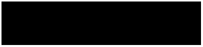 | 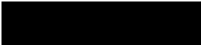 | 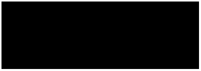 | 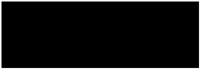 |
| 34 | Nephros™ -LP | Serum-free medium. | Optimized to support growth | Feline kidney cells (CREK) |
| | | supplemented with growth | of kidney cells. | Porcine kidney cells (PK-15) |
| | | factors and | | Buffalo African Green Monkey |
| | | | | kidney cells (BGMK) |
| 35 | HL-1 | Serum-free and chemically | Supports growth of many | Human cell lines U937, Raji, |
| | | defined medium. Ingredients | hybridomas and other cell | MCF-7 (NIH), MCF-7 (MCF), |
| | | include water, a modified | types of lymphoid origin. | NIH ZR-75, COLO 302 HSR, J82, |
| | | DMEM/F12 base, HEPES | | SW1738 SW780, CCL 119, CCL |
| | | buffer, known amounts of | | 213, C91/PL, astrocytoma, |
| | | insulin, transferrin, | | hepatoma, MOLT-3, |
| | | testosterone, sodium selenite, | | MOLT-4, NAMALWA, THP-1 |
| | | ethanolamine, saturated and | | Murine cell lines: |
| | | unsaturated fatty acids and | | BB88, P815, P388D1, WeNi3, |
| | | proprietary stabilizing proteins. | | JLS-V5, GCL2, 70Z-3, 70Z/3.12, |
| | | Without bovine serum albumen | | S49 and variants, RAW309F1.1, |
| | | or other undefined protein | | WeHi7, L5178Y, 1-10, EL4, RL1, |
| | | mixtures. | | BW5147.3, LBRM-3 3, Friend |
| | | | | leukaemia, C57BL6. |
| | | | | Other species cell lines: |
| | | | | CHOK, VERO, MDMK. |
| | | | | Various Hybridomas |
| | | | | Primary cells: |
| | | | | Human peripheral blood T |
| | | | | lymphocytes, |
| | | | | Human blood monocytes |
| | | | | Human fetal adrenal, Mink |
| | | | | lymphocytes. |
| 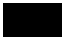 | 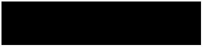 | 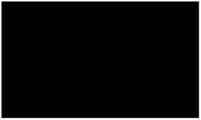 | 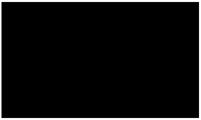 | 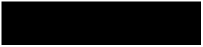 |
| 37 | UltraMDCK™ | Serum-free and low protein | Designed to support growth of | Madin-Darby Canine Kidney cells |
| | | basal medium supplemented | MDCK cells at low and high | (MDCK) |
| | | with only two proteins: bovine | plating densities. Suitable for | |
| | | insulin and bovine transferrin. | large scale bioprocessing and | |
| | | | for in vitro diagnostic use. | |
| 38 | Insect-XPRESS™ | Serum-free and protein-free | Designed to support the | Sf-9 and Sf-21 cells, attached |
| | | medium. Contains L-Glutamine | growth of invertebrate cell | and suspension culture |
| | | | lines derived from Fall Army | |
| | | | worm, Spodoptera frugiperda | |
| | | | (Sf), under attachment | |
| | | | dependent or attachment | |
| | | | independent conditions. | |
| | | | Supports production of | |
| | | | recombinant proteins by cells | |
| | | | infected with viral vectors | |
| | | | such as BEVS. | |

CASCADE BIOLOGICS
www.cascadebio.com
UK suppliers: Patricell Ltd at www.patricell.com

| | | | | |
|---|---|---|---|---|
| 39 | Epilife Medium | Serum-free HEPES based | Primary isolation and long- | Human corneal epithelial cells |
| | | medium. Requires supplementation | tem culture of normal human | Human epidermal keratinocytes. |
| | | with one of the following: | corneal epithelial cells and | |
| | | Epilife Defined Growth | human epidermal keratinocytes. | |
| | | Supplement-contains BSA, | For lab research only. | |
| | | bovine transferrin, | | |
| | | hydrocortisone, rh insulin-like | | |
| | | growth factors. | | |
| | | Human Corneal Growth | | |
| | | Supplemen-contains bovine | | |
| | | pituitary extract, bovine | | |
| | | insulin, hydrocortisone, bovine | | |
| | | transferrin, and mouse | | |
| | | epidermal growth factor. | | |
| | | Human Keratinocyte Growth | | |
| | | factor,-bovine pituitary | | |
| | | extract, bovine insulin, | | |
| | | hydrocortisone, bovine | | |
| | | transferrin, and human EGF. | | |

GIBCO™ INVITROGEN CORPORATION
www.invitrogen.com

| | | | | |
|---|---|---|---|---|
| 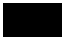 | 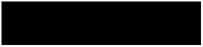 | 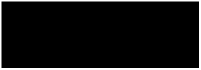 | 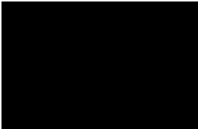 | 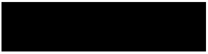 |
| 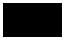 | 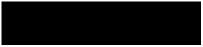 | 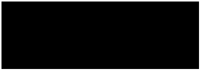 | 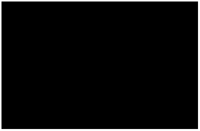 | 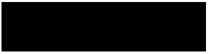 |
| 42 | AIM V | | Ex vivo activation of cytotoxic | Lymphocytes |
| | | | lymphocytes with IL-2 | |
| | | | supplementation Growth of | |
| | | | tumour infiltration (TIL cells) | |
| | | | or cytotoxic T-cells. HIV virus | |
| | | | production. | |
| 43 | CHO-S-SFM II | Low protein (>75 μg/ml) | Growth and production of | Chinese Hamster Ovary (CHO) |
| | | serum-free medium | recombinant proteins in | suspension cultures |
| | | | suspension culture. | |
| 44 | CHO III PFM | Protein-free medium | Growth and production of | CHO cells |
| | | | recombinant proteins in | suspension cultures |
| | | | suspension culture. | |
| 45 | CHO III A PFM | Protein-free medium | Growth and production of | CHO cells |
| | | | recombinant proteins in | |
| | | | adherent culture. | |
| 46 | CHO-A-SFM | Low protein (<250 μg/ml) and | Growth and production of | CHO cells |
| | | serum-free | recombinant proteins in | |
| | | | adherent culture. | |
| 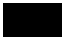 | 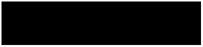 | 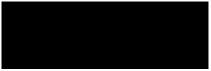 | 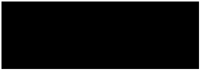 | 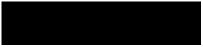 |
| 48 | Drosophila-SFM | Protein-free and serum-free | Growth and maintenance of | Drosophila melanogaster cells, |
| | | medium | adherent or suspension culture. | (D.Mel2, Schoader S2 cells) |
| 49 | Express Five SFM | Protein-free and serum-free | Growth and maintenance of | BT1.TN-5B1-4 insect cells. |
| | | | BT1-TN-5B1-4 insect cells | |
| | | | used for the baculovirus | |
| | | | expression vector system | |
| | | | (BEVS) for adherent or | |
| | | | suspension cultures. Large- | |
| | | | scale production of | |
| | | | recombinant protein expressed | |
| | | | by BEVS. | |
| 50 | Endothelial-SFM | | Growth and maintenance of | Bovine, ovine and porcine, |
| | | | endothelial cells for studying | vascular endothelial cells |
| | | | cell-cell interactions, injury | |
| | | | analysis and atherosclerosis. | |
| 51 | Human Endothelial-STM | Serum-free, supplied with | Growth and maintenance of | Primary and secondary human |
| | | growth factors and attachment | human endothelial cells to | umbilical venous (HUVEC), |
| | | factors (bFGF, EGF, | study signal transduction, | dermal microvascular and arterial |
| | | fibronectin) | cytokine production, and cell | endothelial cells |
| | | | adhesion. | |
| 52 | HepatoZYME-SFM | Serum-free | Maintenance of primary | Primasy human, rat and monkey |
| | | | hepatocyte cells (cytochromes | hepatocytes |
| | | | P450 induction maintained >9 | |
| | | | days) | |
| 53 | Hybridoma-SFM | Serum-free, low protein | Hybridoma growth and | Mouse, human and rat hybridomas |
| | | (<20 μg/ml as insulin and | monoclonal antibody | (AE-1, L5.1, L243, Sp2/0) |
| | | transferrin) medium. Contains | production. | |
| | | Phenol red and surfactant. | | |
| | | Supplementation with a | | |
| | | lipoprotein preparation required | | |
| | | for cholesterol dependent cell | | |
| | | lines (e.g. NSO and derivatives. | | |
| 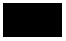 | 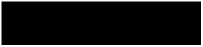 | 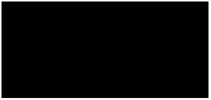 | 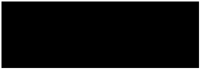 | 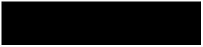 |
| 55 | PFHM-II | Protein-free, serum-free, | Growth and production | Mouse, human and rat hybridomas |
| | | medium. Contains Phenol red | medium for hybridoma cells. | |
| | | and inorganic iron carrier. | | |
| 56 | Defined Keratinocyte-SFM | Low protein serum-free | Growth of primnazy and | Human keratinocytes |
| | | medium, plus insulin, | secondary human | |
| | | epidermal growth factor, and | keratinocytes. | |
| | | fibroblast growth factor. | | |
| 57 | Keratinocyte-SFM | Basal serum-free medium, plus | Studies with dermal | Human keratinocytes |
| | | bovine pituitary extract, human | substitutes, in vitro toxicology | |
| | | recombinant epidermal growth | and gene therapy. Has been | |
| | | factor. | used to cultivate cervical | |
| | | | epithelial cells, and to study | |
| | | | human papillomavirus DNA | |
| | | | transfected cells. May be | |
| | | | suitable for cultivating other | |
| | | | epithelial cells such as human | |
| | | | bronchial epithelial cells. | |
| 58 | Macrophage-SFM | | Growth and maintenance of | Macrophages and monocytes |
| | | | macrophages sad monocytes | |
| | | | (addition of GM-CSF may be | |
| | | | necessary). Demonstration of | |
| | | | macrophage phagocytosis. | |
| | | | Activation of cells to kill | |
| | | | tumour cells with gamma | |
| | | | interferon or | |
| | | | lipopolysaccharide | |
| | | | supplementation. | |
| 59 | Neurobasal™ Medium | Basal medium lacking | Long-term growth of foetal | Foetal neurons |
| | | excitatory amino acids used in | neurons. | |
| | | conjunction with supplements | | |
| | | below to make a complete | | |
| | | serum-free medium. | | |
| 60 | Neurobasal™ A Medium | Basal medium lacking | Long-term growth of postnatal | Adult and postnatal neurons |
| | | excitatory amino acids used in | and adult neurons | (>1 week old) |
| | | conjunction with supplements | | |
| | | below to make a complete | | |
| | | serum-free medium. | | |
| 61 | Neurobasal Supplements | Supplement B27. A serum | Low-density plating and long- | Primary rat embryonic |
| | | substitute | term viability and growth of | hippocampal neurons Primary rat |
| | | | hippocampal and other CNS | neurons from striatum, substantia |
| | | | proliferation. | nigra, septum and cortex |
| | | Supplement B27 AO [B27 | Studies of oxidative damage | |
| | | without any cortex | and rescue, apoptosia, or age- | |
| | | antioxidants] | related neurodegenerative | |
| | | | diseases where free radical | |
| | | | damage to neurons occurs | |
| | | Supplement B-27 Without | Supports growth of CNS | Primary rat embryonic |
| | | Vitamin A | progenitor or stem cells/ | hippocampal neurons, tumour cell |
| | | | | lines of neural origin (PC12, |
| | | | | B104, NIE-115, NS20). |
| | | Supplement N2 | Maintenance and growth of rat | Primary glial cells, tumour cell |
| | | Chemically defined | embryonic hippocampal | lines of glial origin (U-251. Mgsp, |
| | | supplement, containing insulin | neurons. Growth and | C62BD, RN-22), astrocytes, |
| | | (bovine), human transferring | maintenance of tumour cell | microglia, oligodendrocytes. |
| | | (holo), progesterone, | lines of neural origin. | |
| | | putrescine, and selenite. | | |
| | | Supplement G5 | Growth and maintenance of | |
| | | Chemically defined additive | glial cells. | |
| | | containing insulin, human | | |
| | | transferrin, selenite, biotin, | | |
| | | hydrocortisone, FGF and EGF. | | |
| 62 | HIBERNATE-E | With B27 supplement | Short-term maintenance (2 | Embryonic neural tissue |
| | | | days) of neurons in ambient | |
| | | | CO₂ if precultured in | |
| | | | Neurobasal medium with B27. | |
| | | | Long-term storage (1 month) | |
| | | | at 4° C. of prenatal brain tissue. | |
| 63 | HIBERNATE-A | With B27 supplement | Short-term culture (2 days) of | Postnatal and adult neural tissue |
| | | | neurons in ambient CO2 if | |
| | | | precultured with Neurobasal | |
| | | | Medium with B27. Storage of | |
| | | | postnatal and adult brain | |
| | | | tissue | |
| 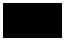 | 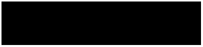 | 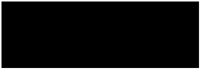 | 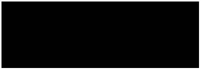 | 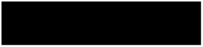 |
| 65 | OPTI-MEM® I reduced Serum | Reduced serum medium, a | Most cells routinely cultured | Various |
| | media | modification of Eagle's | in serum-supplemented | |
| | | Minimum essential medium | medium may be transferred | |
| | | buffered with HEPES and | directly into OPTI-MEM® I | |
| | | sodium bicarbonate, | with a minimum of 50% | |
| | | supplemented with | reduction in serum. Very low | |
| | | hypoxanthine, rhymidine | serum supplement achieved | |
| | | sodium pyruvate, L-glutamine, | with myelomas and derived | |
| | | trace elements and growth | hybridomas, fibroblasts, | |
| | | factors. Phenol red reduced to | epithelial cells or normal and | |
| | | 1.1 mg/L | tumour origins. | |
| 66 | Sf-900 II SFM | Protein-free and serum-free | Growth and maintenance of | Sf9, Sf21, TN368 cells |
| | | | cells used for baculovirus | (Spodoptera frugiperda) |
| | | | expression vector system | |
| | | | (BEVS) for adherent or | |
| | | | suspension culture. Large- | |
| | | | scale production of | |
| | | | recombinant protein expressed | |
| | | | by BEVS. | |
| 67 | STEMPRO-34® SFM | Serum-free medium. Requires | Supports growth of human | Human hematopoietic progenitor |
| | | addition of | hematopoietic progenitor cells | cells (CD34+) from bone marrow, |
| | | L-Glutamine, and Stempro | (CD34+). Optimized using | peripheral blood or neonatal cord |
| | | supplement at time of use. | freshly isolated CD34+ bone | blood |
| | | Extended cell support requires | marrow cells from normal | |
| | | addition of cytokines and | donors. | |
| | | growth factors. | | |
| 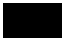 | 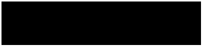 | 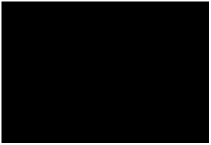 | 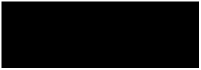 | 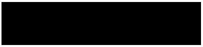 |

HYCLONE
www.hyclone.com

| | | | | |
|---|---|---|---|---|
| 69 | SFM4Mab™ -Utility | Serum-free medium, contains | Designed to support growth of | Multiple hybridoma cell types |
| | | cholesterol and L-Glutamine. | multiple hybridoma cell types. | |
| | | Without Phenol Red or | For production of monoclonal | |
| | | Pluronic F-68 | antibodies for academic and | |
| | | | industrial research, genomics | |
| | | | and proteomics, in vitro | |
| | | | diagnosis, drug target | |
| | | | screening and validation, and | |
| | | | manufacturing of preclinical | |
| | | | lots. | |
| 70 | SFM4Mab™ | Serum-free medium, contains | Designed to increase process | Engineered bybeidoam and |
| | | synthetic cholesterol and | yields for the industrialised | meombinam myelonm cell lines. |
| | | Pluronic F-68. Without Phenol | manufacture of human and | |
| | | Red. Available with or without | humanized recombinant | |
| | | L-Glutamine. | antibodies for therapeutic use | |
| | | | in a variety of engineered | |
| | | | hybridoma and recombinant | |
| | | | myeloma cell lines. | |
| 71 | SFM4CHO™ | Serum-free and protein-free | Designed to increase process | CHO |
| | | medium, with only minimal | yields for the industrial | |
| | | animal derived components | manufacturing of recombinant | |
| | | (cholesterol and cod liver oil), | proteins in a variety of CHO | |
| | | and no components of bovine | cells. Designed to support the | |
| | | origin. Contains Pluronic P-68, | DHFR election/amplification | |
| | | and 2.2 g/l NaHCO₃. Available | system, and to support the GS | |
| | | with or without L-Glutamine. | gene expression system. | |
| | | Without Phenol Red. | | |
| 72 | SFM4CHO™ -Utility | Serum-free and protein-free | Supports growth of multiple | CHO |
| | | medium. Contains Pluronic | CHO cell clones and | |
| | | F68. Available with or without | production of a variety of | |
| | | L-Glutamine. Without Phenol | recombinant proteins for | |
| | | | research, genomics and | |
| | | | proteomics, in vitro diagnosis, | |
| | | | drug target screening and | |
| | | | validation, and manufacturing | |
| | | | of preclinical lots. | |
| 73 | HyQ PF 293 | Serum-free expression | Expression medium for | Spodoptera (Sf9, Sf21) |
| | | medium. | baculovirus production and | Trichoplusia (High Five^{TM}, Tn) |
| | | | recombinant protein | Drosophila |
| | | | purification. Promotes growth | Heliothis |
| | | | of cells from Spodoptera and | Other insect cells |
| | | | Trichoplusia, also Drosophila, | |
| | | | Heliothis and other types of | |
| | | | insect cells. | |

ICN BIOMEDICALS
www.icnbiomed.com

| | | | | |
|---|---|---|---|---|
| 74 | Serum-free Insect Virus | Complete ready to we serum- | Formulated for production of | |
| | Production Medium | free medium. | insect viruses. | |
| | | With L-glutamine | | |
| 75 | Serum-free Medium, | Complete ready-to-use serum- | Formulated for mammalian | Various |
| | Mammalian | free medium. | cell cultures. | |
| | | With L-Glutamine. | | |
| 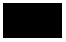 | 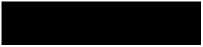 | 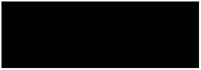 | 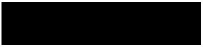 | 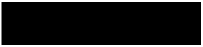 |
| 77 | ICN-CHO | Serum-free, low protein, | Specifically developed for | CHO |
| | | complete medium. | rapid growth to high densities | |
| | | | of CHO cells. | |
| 78 | ICN-Hybridoma | Serum-free, very low protein, | Developed for culture of | Hybridoma |
| | | complete medium. | hybridoma cells and | |
| | | | monoclonal antibody | |
| | | | production. | |
| 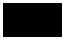 | 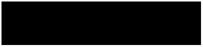 | 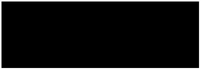 | 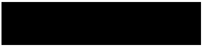 | 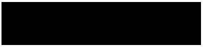 |
| 80 | ICN-MRC-5 | Serum-free, very low protein, | Developed for culture of | MRC-5 |
| | | medium. | MRC-5 cells. | |
| 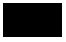 | 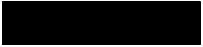 | 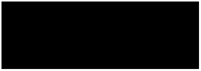 | 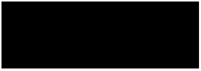 | 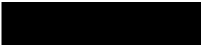 |
| 82 | Cellvation™ | Serum-free and DMSO-free | Cryopreservation. | Various |
| | | cryopreservation medium. | | |
| 83 | TCH™ | Completely defined serum | Developed primarily for | Human: 6T-CEM 20 (T-cell |
| | | replacement. Add to basal | human cells and production of | leukaemia), A375 (malignant |
| | | medium to replace serum. | cell-secreted proteins. Can | melanoma), A549 (lung |
| | | | also be used to culture | carcinoma), Burkitt's Lymphoma. |
| | | | mammalian cells from other | EBV transformed B cells, |
| | | | species. Long-term culture of | Epithelial-human placenta, |
| | | | both anchorage dependent and | malignant, Epithelia-human |
| | | | suspension cultures | placenta benign, HeLa (cervix), |
| | | | | Lymphocytes infected with HIV I |
| | | | | or HIV II, H9 (T-cell lymphoma), |
| | | | | H9/HTLV-IIIB (T-cell, HIV+), |
| | | | | JAR (placenta), Lesch-Nyhan |
| | | | | Syndrome Lymphoblast, Mo (T- |
| | | | | cell leukaemia), MRC-5 (lung), |
| | | | | MT4 (lymphocytes), PC3 (prostate |
| | | | | adenocarcinoma), Primary sensory |
| | | | | neuron precursors, Transformed |
| | | | | human epithelial, U-937 |
| | | | | (histiocytic lymphoma), WI 38 |
| | | | | (lung), WIL-2-729-HF-2 |
| | | | | (B lymphoblast) |
| | | | | Mouse & Hamster, |
| | | | | SP 2/0-Ag-14 (mouse myeloma) |
| | | | | Mouse hybridomas |
| | | | | CHO |
| | | | | CTLL-2 (T-cell mouse tumour) |
| 84 | TCM™ and TCM Insulin Free | Completely defined, serum- | Multi-purpose serum | Human: A375, A549, DU 145, |
| | | free, serum replacement. Add | replacement for a wide range | HEP-2, Human lymphocytes |
| | | to basal medium to replace | of cell types from a variety of | infected with HIV I and HIV II, |
| | | serum, | species. Also primary cell | HeLa, H9, Lesch-Nyhan Syndrome |
| | | | cultures. Supports long-term | Lymphoblast, LNCaP, MCF-7, |
| | | | culture of both anchorage | MEL-14, MRC-5, MT4, NCI-H69, |
| | | | dependant and suspension | neuroblastomas, P3HR-1, Raji, |
| | | | cultures. | SK-MES-1, THPI, WIL-2-729- |
| | | | | HF-2 |
| | | | | Rat & Mouse: B168L6, B16F10, |
| | | | | CTLL-2, PC-12, rat-2 fibroblast, |
| | | | | 3T3, transformed rat fibroblast, |
| | | | | UMR 106 and 108, myelomas and |
| | | | | derived hybridomas (P3X63 |
| | | | | Ag8.653, P3/NSI/1-Ag4-1, |
| | | | | SP 2/O-Ag14). |
| | | | | B95-8, Marmoset EDV |
| | | | | transformed leukocytes |
| | | | | MEL III (rhesus mammary gland) |
| | | | | Vero |
| | | | | Avian (chicken & turkey) |
| | | | | Chicken fibroblasts |
| | | | | QT6 (Quail) |
| | | | | Bovine adrenal cells |
| | | | | Bovine kidney |
| | | | | CHO Chinese hamster ovary |
| | | | | MDCK (canine kidney) |
| | | | | CRFK (feline kidney) |
| | | | | PK15 (porcine kidney) |
| | | | | Swine testicle |
| | | | | Primary Cells: Amphibian renal |
| | | | | cells, |
| | | | | Bovine adrenal cells, aorta |
| | | | | endothelial and kidney, Human |
| | | | | ventricle, Mussel cells, |
| | | | | freshwater glochidia, Neuronal |
| | | | | cultures, Embryonic chick heart, |
| | | | | Prostate carcinoma, |
| | | | | Rabbit cornea, Stomach epithelial |
| | | | | Type II epithelial-lung |

IN VITRO SYSTEMS AND SERVICES
www.ivss.de/en

| | | | | |
|---|---|---|---|---|
| 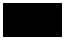 | 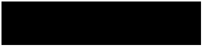 | 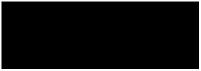 | 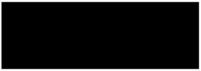 | 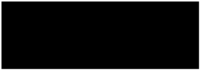 |
| 86 | ProSYSTEM | Serum-free medium based on | Developed for use with | Hybridoma |
| | | RPMI (proSYSTEM ROI) or | MiniPERM, and hollow fiber | |
| | | based on DMEM (proSYSTEM | systems for serum-free or | |
| | | DOI). | reduced serum culture of | |
| | | | hybridoma. Promotes high cell | |
| | | | densities and antibody | |
| | | | concentrations | |

IRVINE SCIENTIFIC
www,irvinesci.com
UK suppliers: Metachem Diagnostics Ltd at www.metachem.co.uk

| | | | | |
|---|---|---|---|---|
| 87 | IS-CHO™ | Serum-free medium, with L- | Optimised to promote long- | CHO cell lines |
| | | Glutamine. Contains bovine- | term, high-density growth of | |
| | | derived components. Without | CHO cells and expression of | |
| | | hypoxanthine or thymidine | recombinant proteins. For use | |
| | | Technical data sheets available | with dihydrofolate reductase | |
| | | on request. | and other selection systems. | |
| 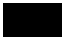 | 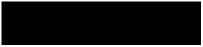 | 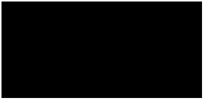 | 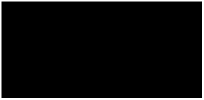 | 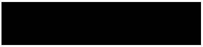 |
| 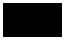 | 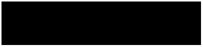 | 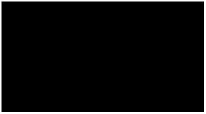 | 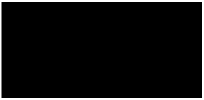 | 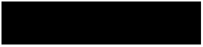 |
| 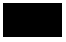 | 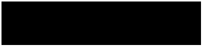 | 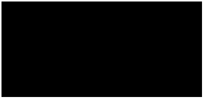 | 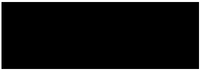 | 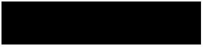 |
| 91 | IS-293 | Serum-free medium, with low | For long-term, high-density | 293 human embryonic kidney cells |
| | | protein content (human | culture of 293 cells, used for | HeLa cells |
| | | transferrin and recombinant | production of adenovirus or | Myelomas |
| | | human insulin). Technical data | recombinant protein | Hybridomas |
| | | sheets available on request. | expression. Optimised to limit | Human breast cancer cells |
| | | | cell aggregation in suspension | |
| | | | cultures. Can also be used for | |
| | | | growth of other cells. | |
| 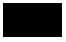 | 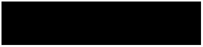 | 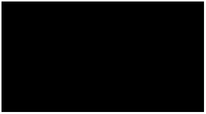 | 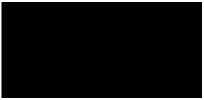 | 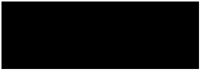 |
| 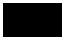 | 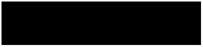 | 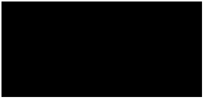 | 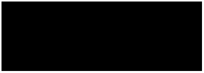 | 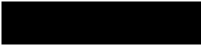 |
| 94 | IS-BAC | Serum-free and protein-free | Supports long-term growth of | Spodoptera frugiperda (SF9 & |
| | | medium | insect cells for production of | SF12) Tricoplusia (BTI-TN-5BI-4 |
| | | | insect virus and recombinant | and high Five^{TM} cells) |
| | | | DNA proteins. | |
| 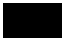 | 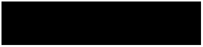 | 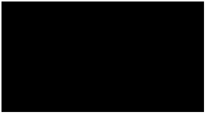 | 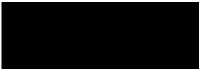 | 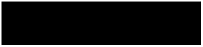 |
| 96 | HB-GRO | Serum-free medium. Technical | Dual formulation with HB- | Human and murine hybridomas |
| | | data available of request. | PRO to support growth and | |
| | | | production phases of | |
| | | | hybridoma culture. | |
| | | | HB-GRO is used for the initial | |
| | | | growth of human and murine | |
| | | | hybridomas from inoculation | |
| | | | to maximal cell density | |
| 97 | HB-PRO | Serum-free and very low | Dual formulation with HB- | Human and marine bybwidomas |
| | | protein medium. Technical date | GRO to support growth and | |
| | | available of request. | production of hybridoma | |
| | | | culture. HB-PRO supports | |
| | | | hybridoma growth during | |
| | | | antibody production phase. | |
| 98 | HB-101 | Serum-free medium kit | | Murine and human hybridomas, |
| | | consisting of basal media and | | myelomas, lymphoblastoid cells, |
| | | lyophilized supplement pack. | | SP2/0 hybrids, P3 hybrids, some |
| | | Technical data available of | | NS-1 cell lines, endothelial |
| | | request. | | cells and murine cell hybrids. |
| 99 | HB-104 | Serum-free kit consisting of | | Human hybridomas, human |
| | | basal media and lyophilized | | myelomas, lymphoblastoid cells, |
| | | supplement pack. Technical | | human T-cells, human B-cells, |
| | | data available on request. | | lymphocytes and LAK cells. |

JRH Biosciences
www.jrhbio.com

| | | | | |
|---|---|---|---|---|
| 100 | EX-CELL™ 301 | Low protein. serum-free | Supports growth and | CHO cells |
| | | medium, Contains L- | maintenance of genetically | |
| | | Glutamine, HEPES, sodium | engineered CHO cells in large- | |
| | | bicarbonate, Pluronic F-68, | scale suspension culture, and | |
| | | glucose, hypoxanthine, | when cells are attached to | |
| | | thymidine, Phenol red. | microcarriers. | |
| | | Animal-derived protein and | | |
| | | hydrolysate. | | |
| 101 | EX-CELL™ 302 | Very low protein, serum-free | Supports CHO cells in | CHO cells |
| | | medium. Contains HEPES, | suspension culture for | |
| | | sodium bicarbonate, Pluronic | expression of antibodies or | |
| | | F-68, glucose. Human | protein products. Appropriate | |
| | | recombinant protein and plant | for use with DHFR or GS | |
| | | derived hydrolysate. | selection systems. | |
| | | Without L-Glutamine, | | |
| | | hypoxanthine, and thymidine. | | |
| 102 | EX-CELL™ 325 PF CHO | Protein-free, serum-free | For growth of CHO cells and | CHO cells |
| | | medium. Contains HEPES, | expression of recombinant | |
| | | sodium bicarbonate, Pluronic | products. Appropriate for use | |
| | | F-68, glucose. Plant derived | with DHFR or GS selection | |
| | | hydrolysate. Without L- | systems. | |
| | | Glutamine, hypoxanthine, and | | |
| | | thymidine. | | |
| 103 | EX-CELL™ 400 | Protein-free and serum-free | Designed for growth of cell | Spodoptera frugiperda, including |
| | | medium. Hydrolysate source | lines from Spodoptera | Sf19 and Sf23 cells |
| | | yeast. Without hypoxanthine | frugiperda. Can be used to | |
| | | and thymidine. | propagate insect cells for | |
| | | | expressing recombinant | |
| | | | products. Can be used in both | |
| | | | suspension and adherent | |
| | | | culture systems. | |
| 104 | EX-CELL™ 405 | Protein-free and serum-free | Optimised for the nutrient and | Trichoplusia ni (High Five™ cells) |
| | | medium. | environmental needs of | |
| | | Hydrolysate source yeast | Trichoplusia ni (High Five™ | |
| | | Without hypoxanthine and | cells). Can be used for | |
| | | thymidine. | expressing recombinant | |
| | | | products using the BEV | |
| | | | system. Can be used in both | |
| | | | suspension and adherent | |
| | | | culture systems. | |
| 105 | EX-CELL™ 420 | Protein-free and serum-free | Optimised for growth of Sf9 | Spodoptera frugiperda Sf9 and |
| | | medium. Contains L- | and Sf21 cells. Can be used in | Sf21 c Schneider S2 (Drosophila) |
| | | Glutamine, Pluronic-F-68, and | both suspension and adherent | cells |
| | | Glucose. Hydrolysate source | culture systems. | |
| | | yeast. | | |
| | | Without hypoxanthine and | | |
| | | thymidine. | | |
| 106 | EX-CELL™ 520 | Serum-free medium containing | Developed for production of | HEK293 cell |
| | | bovine serum albumin, | proteins and adenoviral | |
| | | recombinant growth factors, | vectors using HEK293 cell | |
| | | Pluronic F-68, and glucose. | line in suspension culture. | |
| | | Without L-Glutamine. | | |
| 107 | EX-CELL™ VPRO | Serum-free medium, free of | Developed for long-term | PER.C6™ and related cell lines |
| | | animal protein. | growth of human embryo | |
| | | With Pluronic F-68 and | retinoblast cells (PER.C6™ | |
| | | glucose, hypoxanthine and | and related cell lines) for | |
| | | thymidine. | production of adenovirus. | |
| | | | Cells can be grown as | |
| | | | suspension cultures either in | |
| | | | shaker flasks or roller bottles. | |
| 108 | EX-CELL™ 293 | Serum-free medium, free of | For long-term growth of HEX | HEX 293 cells |
| | | animal protein. | 293 and related cells for | |
| | | With Pluronic F-68 and | adenovirus production in | |
| | | glucose. hypoxanthine and | suspension culture. | |
| | | thymidine. | | |
| 109 | EX-CELL™ MDCK | Serum-free medium, free of | For long-term growth or | MDCK cells |
| | | animal protein. | MDCK and related cells in | |
| | | With Pluronic F-68 and | attachment culture. | |
| | | glucose, hypoxanthine and | | |
| | | thymidine. | | |
| 110 | EX-CELL™ 610-HSF | Low protein, serum-free, | Supports a wide range of cells | Lymphoid and epithelial cells and |
| | | chemically defined medium. | including lymphoid and | B cell hybridomas of murine, rat |
| | | | epithelial cells and B cell | and human origin |
| | | | hybridomas of murine, rat and | |
| | | | human origin. Designed for | |
| | | | production of antibodies. | |
| 111 | EX-CELL™ 620-HSF | Low-protein, serum-free | Supports long-term growth of | Hybridoma and lymphoid cells |
| | | medium. | hybridoma and lymphoid cells, | |
| | | | and is suited for the expression | |
| | | | and isolation of monoclonal | |
| | | | antibodies another protein | |
| | | | products. Suitable for most | |
| | | | cholesterol-dependent | |
| | | | hybridoma cultures. | |

MD BIOSCIENCES
www.mdbiosciences.com

| | | | | |
|---|---|---|---|---|
| 112 | DCCM-1 & DCCM-2 | serum-free media, containing | Culture of myeloma and | Myeloma and hybridoma cells |
| | | BSA. | hybridoma cells, monoclonal | Human lymphocyte cells |
| | | | antibody production; culture of | |
| | | | human lymphocyte cells | |
| | | | (including stimulated and | |
| | | | transformed cells); and virus | |
| | | | production. | |
| 113 | BIOGRO-1 | Serum-free supplement, for | Culture of myeloma and | Myeloma and hybridonu cells |
| | | addition to basal medium. | hybridoma cells, monoclonal | Human lymphocyte cells |
| | | Contains albumin. | antibody production; culture of | |
| | | | human lymphocyte cells | |
| | | | (including stimulated and | |
| | | | transformed cells); and virus | |
| | | | production. | |
| 114 | BIOGRO-2 | Serum-free supplement, for | Culture of myeloma and | Myeloma and hybridonu cells |
| | | addition to basal medium. Very | hybridoma cells, monoclonal | Human lymphocyte cells |
| | | low protein and albumin-free. | antibody production; culture of | |
| | | | human lymphocyte cells | |
| | | | (including stimulated and | |
| | | | transformed cells); and virus | |
| | | | production. | |
| 115 | BIOCHO-1 SFM Base | Serum-free basal medium. | Formulation for adherent CHO | CHO cells |
| | | Contains amino acids, salts, | cells. | CHO-KI and transfected cells |
| | | vitamins, lipids, and trace | | |
| | | elements. Requires addition of | | |
| | | BIOGRO-CHO SFM. | | |
| 116 | BIOCHO-2 SFM Base | Serum-free basal medium. | Formulation for CHO cells in | CHO cells |
| | | Contains amino acids, salts, | suspension | CHO-KI and transfected cells |
| | | vitamins, lipids, and trace | | |
| | | elements. Requires addition of | | |
| | | BIOGRO-CHO SFM. | | |
| 117 | BIOGRO-CHO SFM | Serum-free supplement for | Supplement for addition to | CHO cells |
| | | addition to BIOCHO-1 and | BIOCHO-1 & 2 basal media. | CHO-KI and transfected cells |
| | | BIOCHO-2 SFM. Contains | | |
| | | proteins. | | |

MABIO
www.mabio.net

| | | | | |
|---|---|---|---|---|
| 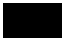 | 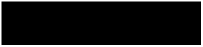 | 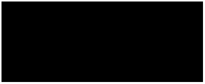 | 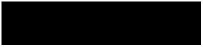 | 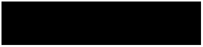 |
| 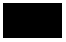 | 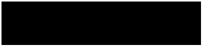 | 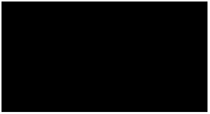 | 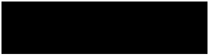 | 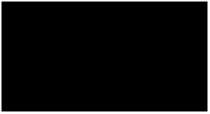 |
| 120 | 00201/RM-B00 | As above (00210/CTM-H00) | Expansion of human, murine, | For human, primate and murine |
| | | but with Bovine Serum | and simian hematopoietic stem | cells: Bone marrow |
| | | Albumin in place of human | and progenitor cells. | Umbilical cord blood |
| | | albumin. | | Normal or mobilized peripheral |
| | | | | blood Mononuclear cells |
| | | | | Selected cells [CD34+ and sub |
| | | | | populations] |
| 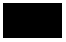 | 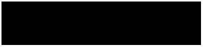 | 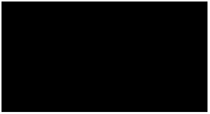 | 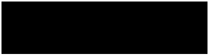 | 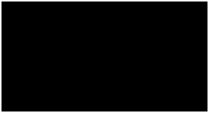 |
| 122 | 00202/RM-B00α | As above (00212/CTM-HOOα) | Expansion of human, murine | Human, primate and murine cells |
| | | but with Bovine Serum | and simian hematopoietic stem | Bone marrow |
| | | Albumin in place of human | and progenitor cells. | Umbilical cord blood |
| | | albumin. | | Normal or mobilized peripheral |
| | | | | blood Mononuclear cells |
| | | | | Selected cells [CD34+ and sub |
| | | | | populations] |
| 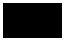 | 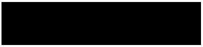 | 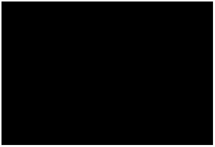 | 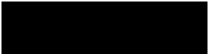 | 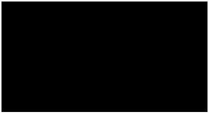 |
| 124 | 00203/RM-B01 | As above (00211/CTM-H01) | Expansion of human | Human cells: |
| | | but with Bovine Serum | hematopoietic primitive | Bone marrow |
| | | Albumin in place of human | progenitor cells. | Umbilical cord blood |
| | | albumin. | | Normal or mobilized peripheral |
| | | | | blood Mononuclear cells |
| | | | | Selected cells [CD34+ and sub |
| | | | | populations] |
| 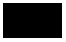 | 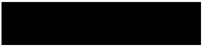 | 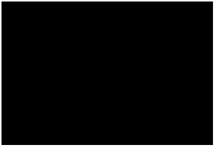 | 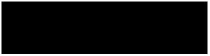 | 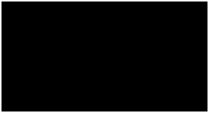 |
| 126 | 00205/RM-B03 | As above (00216/CTM-H03) | Expansion of human | Human cells: |
| | | but with Bovine Serum | megakaryocytic | Bone marrow |
| | | Albumin in place of human | progenitor cells. | Umbilical cord blood |
| | | albumin. | | Normal or mobilized peripheral |
| | | | | blood Mononuclear cells |
| | | | | Selected cells [CD34+ and sub |
| | | | | populations] |
| 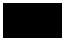 | 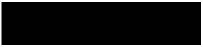 | 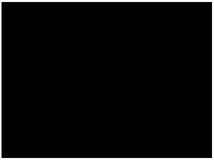 | 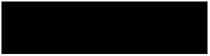 | 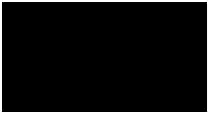 |
| 128 | 00206/RM-B04 | As above (00213/CTM-HO4) | Expansion of human | Human cells: |
| | | but with Bovine Serum | hematopoietic myeloid | Bone marrow |
| | | Albumin in place of human | progenitor cells. | Umbilical cord blood |
| | | albumin. | | Normal or mobilized peripheral |
| | | | | blood Mononuclear cells |
| | | | | Selected cells [CD34+ and sub |
| | | | | populations] |
| 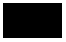 | 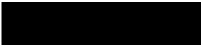 | 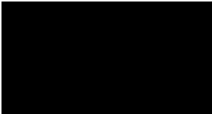 | 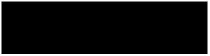 | 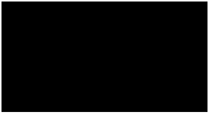 |
| 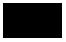 | 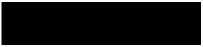 | 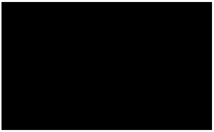 | 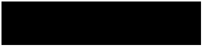 | 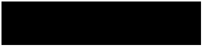 |
| 131 | 00207/RM-B05 | Defined liquid medium. serum- | Expansion of human | Human cells: |
| | | free. Contains bovine serum | hematopoietic erythoid | Bone marrow |
| | | albumin, synthetic iron carrier, | progenitor cells | Umbilical cord blood |
| | | rb-insulin, nucleosides, L- | | Normal or mobilized peripheral |
| | | glutamine, synthetic lipids, α- | | blood Mononuclear cells |
| | | monothioglycerol, synthetic | | Selected cells [CD34+ and sub |
| | | Iscove base medium. Cytokines | | populations] |
| | | rhSCF, rhEPO, RhIL-3, rhIL-6, | | |
| | | rhIL-9, rhIL-11. | | |
| 132 | 00204/RM-B02 | Defined liquid medium. serum- | Expansion of total human | Human cells: |
| | | free. Contains bovine serum | hematopoietic | Bone marrow |
| | | albumin, synthetic iron carrier, | progenitor cells | Umbilical cord blood |
| | | rb-insulin, nucleosides, L- | | Normal or mobilized peripheral |
| | | glutamine, synthetic lipids, α- | | blood Mononuclear cells |
| | | monothioglycerol, synthetic | | Selected cells [CD34+ and sub |
| | | Iscove base medium. Cytokines | | populations] |
| | | rhSCF, rhTPO, rhFLT3-ligand, | | |
| | | rhG-CSF, rhEPO, rhIL- | | |
| | | 1α, rhIL-3, rhIL-6, rhIL-11. | | |

MEDIATECH INC
www.cellgro.com

| | | | | |
|---|---|---|---|---|
| 133 | cellgro Complete™ Serum Free | Complete serum-free medium. | Designed for hybridoma and | Hybridoma |
| | Media | Based on 50/50 mix of | suspension cultures, but also | |
| | | DMEM/F12, with smaller | supports some anchorage | |
| | | percentage of RPMI 1640 and | dependent cell lines. | |
| | | McCoy's 5A. Contains trace | | |
| | | elements, carbohydrates. | | |
| | | vitamins, non-animal protein, | | |
| | | small amoumt of BSA (1 gm/L). | | |
| | | No insulin, transferrin, | | |
| | | cholesterol, growth or | | |
| | | attachment factors. | | |
| 134 | cellgro FREE™ Serum- | Serum and protein-free | Designed for growth of | Mammalian cells, CHO. |
| | free/Protein-free Media | medium, without hormones or | mammalian cell lines. Both | |
| | | growth factors. | adherent and suspension | |
| | | | culture. CHO K-1 require | |
| | | | minimum adaptation into | |
| | | | cellgro-FREE | |
| 135 | Insectgro™ | Serum-free and protein-free | For growth of drosophila S2 | Insect S2 cells |
| | | medium. | cells | |

MEDICULT
www.medi-cult.dk

| | | | | |
|---|---|---|---|---|
| 136 | RenCyte™ System | Completely synthetic, protein- | Consists of four products | Wide range of cells |
| | | free media system based on | RenCyte 1-4 that enable | |
| | | Medi-Cult's patented synthetic | researchers to design their own | |
| | | serum replacement technology. | cell specific culture. | |
| 137 | RenCyte™ BHK | Chemically defined, serum-free | Optimised for culture of baby | BHK |
| | | and protein-free medium. | hamster kidney cells (BHK) | |
| | | Based on modified | and expression of recombinant | |
| | | DMEM/Harris F12. | proteins. | |
| 138 | RenCyte™ Hybridoma | Serum-free and protein-free | Optimised fro culture of | Hybridoma |
| | | medium. | hybridoma and expression of | |
| | | | monoclonal antibodies | |
| 139 | RenCyte™ CHO | Chemically defined, protein- | For cultivation of various | CHO cell lines |
| | | free and serum-free media. | CHO cell lines, both | |
| | | Based on modified | suspension and adherent | |
| | | DMEM/Harris F12. Does not | strains. | |
| | | contain proteins of animal | | |
| | | origin. | | |
| 140 | RenCyte™ Fibroblast | Chemically defined, protein- | For cultivation of fibroblast | Fibroblast cells |
| | | free and serum-free media. | cells for the expression of | |
| | | Based on modified | recombinant and life viral | |
| | | DMEM/Harris F12. No | particles. | |
| | | proteins of animal origin. | | |
| 141 | RenCyte™ CNS | Serum-free and protein-free | Experimentation with | Mammalian brain cells, including |
| | | medium. | mammalian brain cells. | cerebella granule cells, cortical |
| | | | | neurones from rat brain, and PC12 |
| | | | | cells |
| 142 | RenCyte™ Freez | Chemically defined, protein- | Developed to facilitate | Range of cells |
| | | free and serum-free media. | freezing of cells in a serum- | |
| | | Based on modified | free environment. | |
| | | DMEM/Harris F12. | | |

PAN BIOTECH
www.pan-biotech.de/en

| | | | | |
|---|---|---|---|---|
| 143 | Panserin™ -401 | Defined serum-free medium. | All purpose medium for | All purpose medium. |
| | | Based on Iscove's medium, | cultivation of a variety of | |
| | | contains transferrin, bovine | cells. | |
| | | serum albumin, cholseterine, | | |
| | | lipids and trace elements. | | |
| 144 | Panserin™ -501 | Defined serum-free low protein | Monoclonal antibody | Hybridoma |
| | | medium. Based on Iscove's | production. | |
| | | medium, but without bovine | | |
| | | serum albumin. | | |
| 145 | Panserin™ -601 | Defined serum-free, protein- | Useful as a conservation and | Lymphoid cells |
| | | reduced medium. Only protein | production medium for | |
| | | is transferrin. | lymphoid cells. | |
| 146 | Panserin™ -701 | Serum-free medium, based on | Developed for the cultivation | Lymphocytes |
| | | Iscove's with BSA, transferrin, | of lymphocytes targeted for | |
| | | lipids, selected trace elements | the chromosome preparations. | |
| | | and phytohemaglutine. | | |
| 147 | Panserin™ -801 | Serum-free medium, based on | Designed for culture of | Kertatinocytes |
| | | MCDB-153. enriched with | keratinocytes, and to prevent | |
| | | supplements (epidermal growth | explosive growth of | |
| | | factor, insulin, hydrocortisone, | fibroblasts. | |
| | | ethanolamine, phosphate | | |
| | | ethanolamine, BPE) | | |
| 148 | Panserin™ -901 | Serum-free adaptation medium. | Specially designed for | |
| | | | demanding cells. | |

PGC SCIENTIFICS
www.pgcscientifics.com

| | | | | |
|---|---|---|---|---|
| 149 | COS Cell Serum-free System | Two media formulations - one | System consists of two media, | COS cells |
| | | serum-free growth medium and | a serum-free growth medium | |
| | | one protein-free expression | and a protein-free medium for | |
| | | medium. Growth medium | expression of recombinant | |
| | | requires addition of serum | proteins by COS cells. | |
| | | derived proteins (transferrin, | | |
| | | albumin, insulin and | | |
| | | lipoprotein, and other growth | | |
| | | factors) | | |

PAN BIOTECH
www.pan-biotech.de/en

| | | | | |
|---|---|---|---|---|
| 150 | CYTO-SFI | Serum-free supplement to | For cultivation of hybridoma | Hybridoma |
| | | replace or reduce serum | cells | |
| | | requirement. Contains insulin, | | |
| | | BSA, and complex mixture of | | |
| | | trace elements. | | |
| 151 | CYTO-SF2 | Serum-free and protein-free | Especially for protein-free | Hybridoma |
| | | supplement to support growth | cultivation of hybridoma. | |
| | | of hybridoma cells. Contains a | | |
| | | mixture of carbohydrates. | | |
| | | amino acids, vitamins, peptides | | |
| | | and trace elements. May be | | |
| | | used with any basal medium. | | |
| 152 | CYTO-SF3 | Serum-free medium | To support the attachment | NIH-3T3 |
| | | | dependent growth to NIH-3T3 | |
| | | | cells. | |
| 153 | CYTO-SF4 | Serum-free medium contains | Supports growth of human | Human leukaemia cell lines |
| | | insulin, albumin, and | leukaemia cells. | ATCC K562 |
| | | transferrin. Prepared using | | |
| | | Iscove's modified DMEM as a | | |
| | | base formulation. | | |
| 154 | CYTO-SF5 | Serum-free medium | Designed to support | HeLa |
| | | | attachment depended growth | |
| | | | of HeLa cells. For long-term | |
| | | | passage and cryopreservation. | |
| 155 | CYTO-SF7 | Serum-free medium | Designed for growing human | Caco-2 |
| | | | colon adenocarcinoma cell line | |
| | | | Caco-2 | |
| 156 | CYTO-SF10 | Serum-free medium. Contains | For cultivation of African | COS |
| | | all human proteins. | green monkey cell line COS. | |
| | | | Cells may be routinely sub- | |
| | | | cultured and cryopreserved in | |
| | | | the medium | |

PROMOCELL
www.promocell.com

| | | | | |
|---|---|---|---|---|
| 157 | MAM-PF | Chemically defined serum-free | Optimised for the growth of | CHO and BHK cells |
| | | medium, and proteins-free | CHO cells. BHK cells and | |
| | | medium. Without L-glutamate. | expression of recombinant | |
| | | | proteins in suspension culture. | |
| 158 | CHO Express Media | Protein-free & serum-free | For cultivation of CHO cells | CHO cells |
| | SFC-60 & SFC-70 | media (formulation is | producing recombinant | |
| | | confidential) | proteins. | |
| 159 | CHO Express Media | Serum-free media (formulation | For cultivation of CHO cells. | CHO cells |
| | SFC-20, SFC-30, SFC-40 & | is confidential) | SFC-20 & SFC-30 for | |
| | SFC-5O | | anchorage dependent cells. | |
| | | | and SFC-40 & SFC-50 | |
| | | | suspension cultures. | |
| 160 | Hybridoma Growth Medium 7 | Fully defined medium, protein- | Cultivation of hybridoma | Various hybridoma |
| | | free, with or without phenol red | | |
| 161 | Hybridoma Growth Medium 6 | Serum-free medium with L- | Cultivation of hybridoma | Various hybridoma |
| | | glutamate | | |
| 162 | SF-3 BaculoExpress ICM | Serum-free and protein free | Ready to use for cultivation of | Insect cells: |
| | | medium | insect cells | SF9, SF21, High Five™ and |
| | | | | D.Mel-2 |
| 163 | SF-1 BaculoExpress ICM | Serum-free media. Requires | For culture of insect cell lines | Sf9 and Sf21 |
| | | supplementation with lipids | for production of recombinant | |
| | | and Pluronic F68. | proteins in the baculo-virus | |
| | | | expression system | |
| 164 | IP301 BaculoExpress ICM | Chemically defined media, | For culture of insect cell lines | Sf9 and Sf21 |
| | | serum-free media. Requires | for production of recombinant | |
| | | supplementation with lipids | proteins in the baculo-virus | |
| | | and Pluronic P68. | expression system | |
| 165 | TB-1 Clonal Lymphocyte | Defined serum-free medium, | | Lymphocytes |
| | Medium | supplemented with albumin, | | |
| | | transferrin, lecithin, | | |
| | | ethanolamine and some fatty | | |
| | | acids. Contains selenium | | |
| | | pyruvate, potassium nitrate, | | |
| | | additional amino acids, | | |
| | | vitamins and HEPES buffer. | | |
| 166 | VPM-1 Virus Production | Serum-free and protein free | Growth of VERO cells. | VERO cells |
| | Medium | medium. | | |

QUALITY BIOLOGICAL, Inc.
www.qualitybiological.com
QBS media is also sold by Sigma-Aldrich Co.

| | | | | |
|---|---|---|---|---|
| 167 | QBSF-51 | Serum-free, low protein | Designed to support growth of | COS, DA-1, HL-60, Jurkat, K-562, |
| | | medium consisting of Iscove's | transformed cells in | MLA, 144, Molt 4, Namalwa, |
| | | (IMDM) base, with HEPES, | suspension. Has also been | Neuro 2A, NIH 3T3, Raji, Wehi. |
| | | sodium bicarbonate, BSA, | used for colony selection and | |
| | | human transferrin, rh insulin, | focus formation of NIH 3T3 | |
| | | L-Glutamine. | cells. | |
| 168 | QBSF-52 | Serum-free, low protein | Designed to support growth of | Hybridoma |
| | | medium of Iscove's (IMDM) | hybridomas and production of | ACT IV |
| | | base, with HEPES, sodium | monoclonal antibodies. | |
| | | bicarbonate, lithium chloride. | | |
| | | BSA, human transferrin, rh | | |
| | | insulin. L-Glutamine. | | |
| 169 | QBSF-55 | Serum-free, low protein | Designed to support growth of | Hybridoma |
| | | medium consisting of Iscove's | hybridomas and production of | Sp2/0-Ag14 |
| | | (IMDM) base, with HEPES, | monoclonal antibodies. | NS-1 |
| | | sodium bicarbonate, lithium | | |
| | | chloride, bovine serum | | |
| | | albumin, human transferrin, | | |
| | | human recombinant insulin, | | |
| | | cholesterol, L-Glutamine. | | |
| 170 | QBSF-56 | Serum-free, low protein | Designed to support human | Human blood lymphocytes |
| | | medium consisting of Iscove's | peripheral blood blastogenesis | Sp2/0-Ag14 |
| | | (IMDM) base, with HEPES, | and cell cultures. Has been | Sp2 SS1 |
| | | sodium bicarbonate, bovine | used to support proliferation of | Sp2 SA3 |
| | | serum albumin, human | primary cell cultures. Has been | Sp2 SA5 |
| | | transferrin, human recombinant | successfully used to grow a | Sp2 MAI |
| | | insulin, cholesterol. L- | number of immunoglobulin | U-937 |
| | | Glutamine. | secreting clones and other cell | |
| | | | lines. | |
| 171 | QBSF-58 | Serum-free, low protein | Designed for expansion of | Human and murine bone marrow |
| | | medium consisting of Iscove's | human and murine bone | cells |
| | | (IMDM) base, with HEPES, | marrow cells. | |
| | | sodium bicarbonate, bovine | | |
| | | serum albumin, human | | |
| | | transferrin, hr insulin, | | |
| | | cholesterol, L-Glutamine. | | |
| 172 | QBSF-59 & | Serum-free media | Designed to support human | CD34+ cells derived front normal |
| | QBSF-60 | | hematopoietic stem/progenitor | bone marrow, cord blood, and |
| | | | cells. | peripheral blood stern cells. |

ROCHE
www.roche-applied.science.com

| | | | | |
|---|---|---|---|---|
| 173 | Nutridoma-CS | Defined medium supplement | Optimised for freshly fused | Hybridomas derived from SP 2/0, |
| | | that can be used to completely | hybridomas during selection | P3X63Ag8.653 and NS-1 myeloma |
| | | replace serum in cell culture | and cloning procedures in | cell lines. |
| | | medium (e.g. RPMI 1640). | serum-free cell culture. | |
| | | Composed of albumin, insulin, | | |
| | | transferrin, cytokines, | | |
| | | cholesterol source and other | | |
| | | defined organic and inorganic | | |
| | | compounds. | | |
| 174 | Nutridoma-HU | Biochemically defined serum- | Supports the growth of most | Human myeloma and hybridoma |
| | | free supplement that can be | lymphoblastoid, myeloma and | cell lines and primary cultures of |
| | | used to completely replace | hybridoma cell lines, as well | human lymphoid cells. |
| | | serum in cell culture medium | as primary lymphoid cell | |
| | | (e.g. DMEM/RPMI 1640). | cultures. | |
| | | Composed of albumin, insulin, | | |
| | | transferrin, and other defined | | |
| | | organic can inorganic | | |
| | | compounds. Most proteins are | | |
| | | of human origin. | | |
| 175 | Nutridoma-NS | Biochemically defined, serum- | Supports the growth of most | NS-1 and P3X63-Ag8.653 |
| | | free supplement that can be | lymphoblastoid. myeloma and | myeloma cell lines and their fusion |
| | | used to completely replace | hybridoma cell lines, as well | derived hybridomas. CHO cells |
| | | serum in cell culture medium | as primary lymphoid cell | |
| | | (e.g. high glucose | cultures. Occasionally | |
| | | DMEM/Ham's F12). | successful with non-lymphoid | |
| | | Composed of albumin, insulin, | cell lines. | |
| | | transferrin, and other defined | This NS formulation meets | |
| | | organic can inorganic | nutritional requirements for | |
| | | compounds. Plus a cholesterol | cell lines having a deficiency | |
| | | source. | in the biosynthetic pathway for | |
| | | | cholesterol | |
| 176 | Nutridoma-SP | Biochemically defined, serum- | Supports murine myelomas | Murine Sp/2/0 myeloma cell lines |
| | | free supplement that can be | and hybridomas that have | and their fusion derived |
| | | used to completely replace | intact cholesterol biosynthesis | hybridomas. Neural explants |
| | | serum in cell culture medium | pathway. Also for culture of | |
| | | (e.g. DMEM/RPMI 1640). | various other cell types, | |
| | | Composed of albumin, insulin, | including neural explants. | |
| | | transferrin, and other defined | | |
| | | compounds. | | |

STEMCELL TECHNOLOGIES
www.stemcell.com

| | | | | |
|---|---|---|---|---|
| 177 | StemSpan™ SFEM | Serum-free expansion medium. | Developed for the culture and | Human hematopoietic progenitor |
| | | Components include bovine | expansion of human | cells |
| | | serum albumin, human | hematopoietic progenitor cells. | |
| | | recombinant insulin, human | Optimised and tested using | |
| | | transferrin (iron-saturated), 2- | CD34+ enriched cell | |
| | | Mercaptoehtanol, L-Glutamine, | populations from normal | |
| | | Iscove's MDM. | donors. | |
| 178 | StemSpan™ H3000 | Serum-free defined medium | Developed for culture of | Human hematopoietic cells |
| | | containing only pre-tested | human hematopoietic cells | |
| | | human-derived or recombinant | | |
| | | human proteins. Requires | | |
| | | supplementing with | | |
| | | recombinant cytokines. | | |
| 179 | StemSpan™ BIT 9500 | Serum substitute for use in | For use where media of | Human and murine hematopoietic |
| | | place of fetal bovine serum. | defined composition is | progenitor cells |
| | | Contains pre-tested batches of | required. For use in | |
| | | bovine serum albumin, human | methylcellulose-based colony | |
| | | recombinant insulin and human | assay of for expansion | |
| | | transferrin (BIT). Plus | medium for human or murine | |
| | | Iscove's MDM. | hematopoietic progenitors. | |

SIGMA-ALDRICH Co.
www.sigmaaldrich.com

| | | | | |
|---|---|---|---|---|
| 180 | CHO Serum-free Medium | Serum-free medium containing | Optimized for use in | CHO cells |
| | | inorganic salts, HEPES, | recombinant protein | |
| | | sodium bicarbonate, essential | expression and production in | |
| | | and non-essential amino acids, | Chinese hamster Ovary (CHO) | |
| | | vitamin, bovine serum | cell systems. | |
| | | albumin, human transferrin, | | |
| | | fetal bovine fetuin (USA | | |
| | | source) trace elements, phenol | | |
| | | red, Pluronic F-68, and other | | |
| | | organic compounds. | | |
| | | Without L-glutamine, | | |
| | | antibiotics, and antimycotics. | | |
| | | Also no hypoanthine or | | |
| | | thymidine. | | |
| 181 | Hybridoma Medium, Serum- | Serum-free medium containing | Supports high viable cell | Hybridoma |
| | free | inorganic salts, essential and | densities and high antibody | |
| | | non-essential amino acids, | productivity over extended | |
| | | vitamins, sodium bicarbonate, | culture periods of 60 days or | |
| | | HEPES, trace elements, fatty | greater. Suitable for cloning | |
| | | acids and other organics. | and fusion applications. | |
| | | Contains low concentrations of | | |
| | | bovine serum albumin and | | |
| | | human transferrin. Does not | | |
| | | contain phenol red, L- | | |
| | | glutamine, antibiotics, and | | |
| | | antimycotics. | | |
| 182 | Serum-free and Protein-free | Based on a modification of | Designed to support growth of | Hybridoma |
| | Hybridoma Medium | Ham's Nutrient Mixture F-12 | hybridomas, but may not | |
| | | containing additional | support myelomas which | |
| | | components and MOPS | require cholesterol | |
| | | buffering system. Full formula | | |
| | | available. | | |
| 183 | MDBK-GM-SF | Serum-free, low protein | This medium together with the | MDBK cells |
| | Madin-Darby Bovine Kidney | medium containing inorganic | protein-free version below, are | |
| | Growth Medium Serum-free | salts, HEPES and sodium | designed to support optimal | |
| | | carbonate buffers, essential and | growth of MDBK cells for | |
| | | non-essential amino acids, | production of viruses useful | |
| | | vitamins, recombinant human | for vaccines, in two phases: | |
| | | insulin and growth factors, | the growth phase and the high- | |
| | | fetuin, transferrin, other | density maintenance and virus | |
| | | organic compounds and trace | production phase. | |
| | | elements. Without L-glutamine. | MDBK-GM-SF is designed to | |
| | | | support growth of MDBK | |
| | | | cells before transfer into the | |
| | | | protein-free medium below. | |
| | | | Recommended for use with | |
| | | | roller bottle and microcarrier | |
| | | | bead based bioreactor system. | |
| 184 | MDBK-MM-PF | Serum-free, protein-free | Designed far maintenance of | MDBK cells |
| | Madin-Darby Bovine Kidney | medium containing inorganic | MDBX cells in high densities | |
| | Maintenance Medium Protein- | salts, HEPES and sodium | over extended periods of time, | |
| | free | carbonate buffers, essential and | and for virus production. | |
| | | non-essential amino acids, | Recommended for use with | |
| | | vitamins, recombinant human | roller bottle and micro carrier | |
| | | insulin and growth factors, | bead based bioreactor system. | |
| | | fetuin, transferrin, other | | |
| | | organic compounds and trace | | |
| | | elements. Without L-glutamine. | | |
| 185 | MDCK-SF | Serum-free medium containing | Designed to support the | MDCX cells |
| | Madin-Darby Canine Kidney | inorganic, salts, essential and | growth of Madin-Darby canine | |
| | Medium, Serum-free | non-essential amino acids, | kidney cells that support the | |
| | | vitamins, recombinant human | production of viruses useful as | |
| | | insulin and growth factors, | vaccines. | |
| | | other organic compounds and | | |
| | | trace elements. Contains bovine | | |
| | | serum albumin. Without L- | | |
| | | glutamine | | |
| 186 | MDCK-PF | Serum-free medium containing | Designed to support the | MDCK cells |
| | Madin-Darby Canine Kidney | inorganic salts, essential and | growth of Madin-Darby canine | |
| | Medium, Protein-free | non-essential amino acids, | kidney cells that support the | |
| | | vitamins, recombinant human | production of viruses useful as | |
| | | insulin and growth factors, | vaccines. | |
| | | other organic compounds and | | |
| | | trace elements. Without L- | | |
| | | glutamine. | | |
| 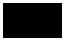 | 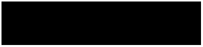 | 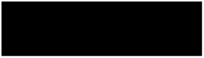 | 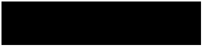 | 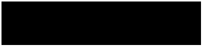 |
| 188 | Serum-free Insect Medium-1 | Serum-free and protein-free | Developed for production of | Insect cells |
| | Protein-free | medium with L-glutamine and | recombinant proteins. | |
| | | sodium bicarbonate. Based on | | |
| | | IPL-41 medium originally | | |
| | | developed by Weiss et al at the | | |
| | | USDA Insect Pathology | | |
| | | Laboratory. Contains inorganic | | |
| | | salts, amino acids, vitamins, | | |
| | | other organic compounds and | | |
| | | trace elements. | | |
| 189 | Serum-free Insect Medium-2 | Serum-free and low-protein | Developed for production of | Insect cells |
| | Low-protein | medium with L-glutamine and | recombinant proteins. | |
| | | sodium bicarbonate. Based on | | |
| | | IPL-41 medium originally | | |
| | | developed by Weiss et al at the | | |
| | | USDA Insect Pathology | | |
| | | Laboratory. Contains inorganic | | |
| | | salts, amino acids, vitamins, | | |
| | | other organic compounds and | | |
| | | trace elements. | | |
| 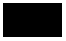 | 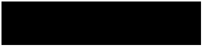 | 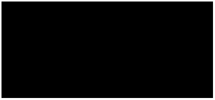 | 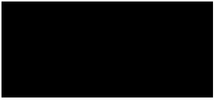 | 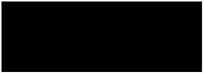 |
| 191 | Stemline™ Serum-free | Semi-solid serum-free medium | For assay of human | Human hematopoietic cells |
| | Methylcellulose Medium | consists of Iscove's Modified | clonogenic hematopoietic | |
| | Without Growth Factors, | Dulbecco's Medium, | progenitor cells isolated from | |
| | Human | methylcellulose, bovine serum | bone marrow or other | |
| | | albumin, L-Glutamine, 2- | hematopoietic cells. For | |
| | | Mercaptoethanol, rh insulin, | enumeration and evaluation of | |
| | | human transferrin, iron- | stem cell derived progeny | |
| | | saturated. Does not contain | characterised as colony | |
| | | erythropoietin or other | forming units. | |
| | | cytokines. | | |
| 192 | Stemline™ Serum-free | Semi-solid serum-free medium | For assay of murine | Mouse hematopoietic cells |
| | Methylcellulose Medium | consists of Iscove's Modified | clonogenic hematopoietic | |
| | Without Growth Factors, | Dulbecco's Medium, | progenitor cells isolated from | |
| | Mouse | pharmaceutically grade | murine bone marrow or other | |
| | | methylcellulose, bovine serum | hematopoietic cells. For | |
| | | albumin, L-Glutamine, 2- | enumeration and evaluation of | |
| | | Mercaptoethanol, rh insulin, | stem cell derived progeny | |
| | | human transferrin, iron- | characterised as colony | |
| | | saturated. Does not contain | forming units. | |
| | | erythropoietin or other | | |
| | | cytokines. | | |
| 193 | Cell Freezing Medium-DMSO, | Serum-free medium for | Designed to protect and | |
| | Serum-Free | cryopreservation, containing | preserve cells during frozen | |
| | | 8.7% DMSO in MEM | storage. | |
| | | supplemented with | | |
| | | methylcellulose. | | |
| 194 | Cell Freezing Medium Serum- | Serum-free medium for | Designed to protect and | |
| | Free | cryopreservation, without | preserve cells during frozen | |
| | | DMSO | storage. | |
| 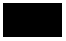 | 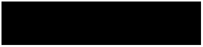 | 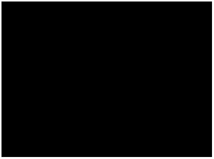 | 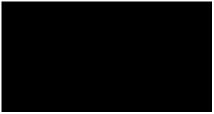 | 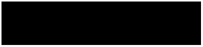 |
| 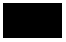 | 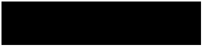 | 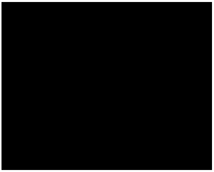 | 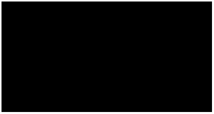 | 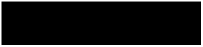 |

BSA = bovine serum albumin
EGF = epidermal growth factor
rhEGF = human recombinant epidermal growth factor
FGF = fibroblast growth factor
hrbFGF = human recombinant basic fibroblast growth factor
GM-CSF = granulocyte-macrophage colony-stimulating factor (can be human recombinant)

TABLE II

| | Species of | | | |
|---|---|---|---|---|
| Antigenic Determinant | Hybridoma | Isotype | Name | Catalog No. |
| Abelson murine leukemia virus, 16-kDa antigen | rat/mouse | IgG2a | CDR1 | HB-213 |
| abl oncogene peptide, synthetic | mouse | IgG1 and IgG2b | 310-29F7 | CRL-2656 |
| abl oncogene peptide, synthetic | mouse | IgG1 and IgG2b | 311-3D4 | CRL-2657 |
| abl oncogene peptide, synthetic | mouse | IgG1 | 312-13E8 | CRL-2658 |
| Acetylcholine receptor (AChR) alpha subunit | rat | IgG2a | mAb64 | HB-8987^{†} |
| Acetylcholine receptor (AChR) alpha subunit | rat/mouse | IgG1 | mAb 35 | HB-8857^{†} |
| Acetylcholine receptor (AChR) alpha subunit | rat/mouse | IgG1 | mAb35 | TIB-175 |
| Acetylcholine receptor, neuronal, chicken | rat/mouse | IgG2a | mAb 270 | HB-189 |
| Acetylcholine receptor, neuronal, rat | rat/mouse | IgG2a | mAb 270 | HB-189 |
| Acetylcholinesterase, human | mouse | IgG1 | AE-1 | HB-72 |
| Acetylcholinesterase, human | mouse | IgG1 | AE-2 | HB-73 |
| Acid phosphatase, prostatic (PAP), human | mouse | IgG1 | RLTM01 | HB-8526^{†} |
| Acid phosphatase, prostatic (PAP), human | mouse | IgG1 | RLTM02 | HB-8523^{†} |
| Actin | mouse | IgG1 | ACT I | HB-80 |
| Actin | mouse | IgG1 | ACT IV | HB-81 |
| Actinin, alpha | mouse | IgM | G-3-5 | CRL-2252 |
| Addressin, mucosal vascular, mouse | rat/mouse | IgG2a | MECA-89 | HB-292 |
| Addressin, peripheral node, mouse | rat/mouse | IgM | MECA-79 | HB-9479^{†} |
| Adenocarcinoma, colon, human | mouse | IgG1 | CLT 85 | HB-8240^{†} |
| Adenocarcinoma, colon, human | mouse | IgG3 | HT 29/36 | HB-8248^{†} |
| Adenovirus group-specific antigen | mouse | IgG2a | 2Hx-2 | HB-8117^{†} |
| Agrobacterium tumefaciens biovar 3 | mouse | IgG1 | F21-1D3G7C8 | HB-9463^{†} |
| Aldosterone | mouse | IgG1 | A2E11 | CRL-1846 |
| Alpha fetoprotein (AFP), human | mouse | IgG1; kappa | OM 3-1.1 | HB-134 |
| Alpha-1,3-dextran | mouse | IgA; lambda | J558 | TIB-6 |
| Alveolar surfactant protein (ASP) | mouse | IgG | DS-1 | HB-8906^{†} |
| Alveolar surfactant protein (ASP) | mouse | IgG1 | DS-3 | HB-8651^{†} |
| Alveolar surfactant protein (ASP) | mouse | IgG1 | DS-5 | HB-8653^{†} |
| Alveolar surfactant protein (ASP) | mouse | IgG1 | DS-6 | HB-8652^{†} |
| Amylase, salivary, human | mouse | IgG2a | 110-5 | HB-8984^{†} |
| Angiotensin-converting enzyme (ACE) | mouse | IgM | α-ACE 3.1.1 | HB-8191^{†} |
| Annexin I, human | mouse | IgG1 | EH17a | CRL-2209 |
| Annexin I, human | mouse | IgG1 | EH7a | CRL-2194 |
| Annexin II, human | mouse | IgG1 | EH7a | CRL-2194 |
| Antigen-dependent killer (K) cells, human | mouse | IgM; kappa | HNK-1 | TIB-200 |
| AP-2 adaptor protein of clathrin coated vesicles | mouse | IgG1 | AP.6 | CRL-2227 |
| Apolipoprotein A-I (Apo-A-I), human | mouse | IgG1 | A5.4 | CRL-2275 |
| Apolipoprotein E (ApoE), human | mouse | IgG1 | WU E-14 | CRL-2255 |
| Apolipoprotein E (ApoE), human | mouse | IgG1 | WU E-4 | CRL-2247 |
| Asialo GM1 | mouse | IgM | SH-34 | CRL-2405 |
| Asialo GM2 | mouse | IgM | 2D4 | TIB-185 |
| Astrocyte protein, human | mouse | IgM | J1-31 | CRL-2253 |
| Astrocyte, rat | mouse | IgG2a | RAN-2 | TIB-119 |
| Astrocytoma cell line, human | mouse | IgG2a | G253 | HB-9706^{†} |
| Astrocytoma cell line, human | mouse | IgG1 | K117 | HB-8553^{†} |
| Astrocytoma cell line, human | mouse | IgG1 | S5 | HB-9255^{†} |
| Astrovirus group antigen | mouse | IgG1 | 7F2-6D4-8E7 | HB-11945^{†} |
| ATPase, rat (Na, K dependent) | mouse | IgG1 | 9-A5 | CRL-1844 |
| ATPase, rat (Na, K dependent) | mouse | IgG1 | 9-B1 | CRL-1845 |
| Autocrine growth factor, 15 kDa, human | mouse | IgM | CBL-1 | HB-8214^{†} |
| B cell antigen (p50), mouse | rat/mouse | IgM | RA3-2C2/1 | TIB-145 |
| B cell derived malignancies, human | mouse | IgG2a | Lym-1 | HB-8612^{†} |
| B cell growth factor 1, mouse | rat/mouse | IgG1 | 11B11 | HB-188 |
| B cell precursors, mouse | rat/mouse | IgG2b | 14.8 | TIB-164 |
| B cell stimulatory factor 1, mouse | rat/mouse | IgG1 | 11B11 | HB-188 |
| B cells, bovine | mouse | IgG2a | CC56 | HB-273 |
| B cells, human | mouse | IgG2a | Lym-1 | HB-8612^{†} |
| B lymphocytes, mouse | rat/mouse | IgM | J11d.2 | TIB-183 |
| B220, mouse | rat/mouse | IgM | RA3-3A1/6.1 | HB-146 |
| B7.1, mouse | hamster/mouse | IgG | 16-10A1 | HB-301 |
| B7.1, mouse | rat/mouse | IgG2a | IG10 | CRL-2223 |
| B7.2, mouse | rat/mouse | IgG2b | 2D10 | CRL-2226 |
| B7.2, mouse | rat/mouse | IgG2a | GL1 | HB-253 |
| Basal cells (skin), human | mouse | IgG1 | VM-2 | HB-8530^{†} |
| BCGF-1, mouse | rat/mouse | IgG1 | 11B11 | HB-188 |
| Bicoid (bcd) protein, Drosophila melanogaster | mouse | IgG1 | bcd mab23 | CRL-2107 |
| Blood group A antigen | human | IgM | HAA1 | HB-8534^{†} |
| Bluetongue virus VP7 | mouse | IgG2b | 7D3A.2 | CRL-1886 |
| Bluetongue virus VP7 | mouse | IgG2a | 8A3B.6 | CRL-1875 |
| Bluetongue virus VP7 | mouse | IgG2b | 8B1B.1 | CRL-1877 |
| Bovine herpesvirus 1 (BHV-1) | bovine/mouse | IgG1 | αBL5C2.870005 | HB-9907^{†} |
| Bovine herpesvirus 1 (BHV-1) | bovine/mouse | IgG1 | αBL5C2.870009 | HB-9908^{†} |
| Bovine herpesvirus 1 (BHV-1) | bovine/mouse | IgG1 | αBL5C2.870016 | HB-9909^{†} |
| Bovine herpesvirus 1 (BHV-1) | mouse | IgG1 | 1B8-F11 | CRL-1852 |
| Bovine herpeavirus 1 (BHV-1) | mouse | IgG2b | 2H6-C2 | CRL-1853 |
| Bp35 (B cell antigen), human | mouse | IgG2a | IF5 | HB-9645^{†} |
| Bp50 (B cell antigen), human | mouse | IgG1 | G28-5 | HB-9110^{†} |
| Breast cancer cells, human | mouse | IgG1 | 317G5.C1D3 | HB-8691^{†} |
| Breast cancer cells, human | mouse | IgG2a | 454C11 | HB-8484^{†} |
| Breast cancer cells, human | mouse | IgG1 | 520C9 | HB-8696^{†} |
| Breast cancer cells, human | mouse | | 650E2-2B12 | HB-10812^{†} |
| BSF-1, mouse | rat/mouse | IgG1 | 11B11 | HB-188 |
| Bubonic plague bacillus | mouse | IgA | F1-3G8-1 | HB-192 |
| C3d receptor (CR2), human | mouse | IgG2a; kappa | THB-5 | HB-135 |
| Canine adenovirus type 1 (CAV-1) | mouse | IgG1 | 2E10-H2 | CRL-1812 |
| Canine adenovirus type 2 (CAV-2) | mouse | IgG2a | 4H1-A7 | CRL-1813 |
| Canine distemper virus (CDV) | mouse | IgG1 | CDC 1C42H11 | HB-216 |
| Carcinoembryonic antigen (CEA) | mouse | IgM | 1116NS-3d | CRL-8019 |
| Carcinoembryonic antigen (CEA) | mouse | IgG1; kappa | T84.66A3.1A.1F2 | HB-8747^{†} |
| Carcinoma cells, human | mouse | IgG3 | KC-4G3 | HB-8709^{†} |
| Carcinoma cells, human | mouse | IgM | KC-4M1 | HB-8710^{†} |
| Carcinoma associated antigen, heat stable, human | mouse | IgG2a | AS 33 | HB-8779^{†} |
| CD1, bovine | mouse | IgG2a | CC20 | HB-267 |
| CD1, human | mouse | IgG1 | OKT 6 | CRL-8020 |
| CD1, pig | mouse | IgG2a; kappa | 76-7-4 | HB-140 |
| CD1.1, mouse | rat/mouse | IgG2b; kappa | 15C6 | HB-326 |
| CD1.1, mouse | rat/mouse | IgG2b; kappa | 15F7 | HB-322 |
| CD1.1, mouse | rat/mouse | IgG2b; kappa | 19F8 | HB-321 |
| CD1.1, mouse | rat/mouse | IgG1; kappa | 20H2 | HB-323 |
| CD1.1, mouse | rat/mouse | IgG2b | 4C4 | HB-327 |
| CD1w2, bovine | mouse | IgG2a | CC20 | HB-267 |
| CD2, bovine | mouse | IgG1 | CC42 | HB-272 |
| CD2, bovine | mouse | IgG2a | IL-A42 | CRL-1870 |
| CD2, human | mouse | IgG2a | 35.1 | HB-222 |
| CD2, human | mouse | IgG1 | OKT 11 | CRL-8027 |
| CD2, human | mouse | IgG1 | TS2/18.1.1 | HB-195 |
| CD2, sheep | mouse | IgG2a | 36F-18C | HB-285 |
| CD3 epsilon chain, human | mouse | IgG2b | BC3 | HB-10166^{†} |
| CD33, human | mouse | IgG2a | M195 | HB-10306^{†} |
| CD34, human | mouse | IgG1; kappa | AC133.1 | HB-12346^{†} |
| CD35, human | mouse | IgG1; kappa | Mab 543 | HB-8592^{†} |
| CD38, human | mouse | IgG1 | OKT 10 | CRL-8022 |
| CD38, human | mouse | IgG1 | THB-7 | HB-136 |
| CD40 ligand (CD154, CD40L), human | mouse | IgG1 | hCD40L-M90 | HB-12055^{†} |
| CD40 ligand (CD154, CD40L), human | mouse | IgG1 | hCD40L-M91 | HB-12056^{†} |
| CD40 ligand, human | mouse | IgG2a | 5c8 | HB-10916^{†} |
| CD40 ligand, mouse | hamster/mouse | IgG | MR1 | CRL-2580 |
| CD40, human | mouse | IgG2b | 3A8 | HB-12024^{†} |
| CD40, human | mouse | IgG1 | G28-5 | HB-9110^{†} |
| CD44, human | mouse | IgG2a | Hermes-3 | HB-9480^{†} |
| CD44, mouse | rat/mouse | IgG1 | KM114 | TIB-242 |
| CD44, mouse | rat/mouse | IgG1 | KM201 | TIB-240 |
| CD44, mouse | rat/mouse | IgG2a | KM703 | CRL-1896 |
| CD44, mouse | rat/mouse | IgG2a | KM81 | TIB-241 |
| CD44, mouse | rat/mouse | IgG2a | LYK-12 | HB-316 |
| CD44, mouse | rat/mouse | IgG2a | LYK-16 | HB-319 |
| CD44, mouse | rat/mouse | IgG1 | LYK-5 | HB-310 |
| CD44, mouse, isoforms expressing variable | rat/mouse | IgG1 | LYK-1 | HB-306 |
| exon V10 | | | | |
| CD44, mouse, isoforms expressing variable | rat/mouse | IgG1 | LYK-7 | HB-311 |
| exon V10 | | | | |
| CD44, mouse, isoforms expressing variable | rat/mouse | IgG2a | LYK-8 | HB-312 |
| exon V10 | | | | |
| CD44, mouse, isoforms expressing variable | rat/mouse | IgG2a | LYK-9 | HB-313 |
| exon V10 | | | | |
| CD44, v4 variant, human | mouse | IgG2a | FW11-10-3 | HB-257 |
| CD44, v6 variant, human | mouse | IgG2a | FW11-9-2 | HB-256 |
| CD44, v9 variant, human | mouse | IgG1 | FW11-24-17-36 | HB-258 |
| CD45, human | mouse | IgG2a | 4B2 | HB-196 |
| CD45, human | mouse | IgG2a | 9.4 | HB-10508^{†} |
| CD45, human | mouse | IgG2a; kappa | GAP 8.3 | HB-12 |
| CD45, mouse | rat/mouse | IgG2b | M1/89.18.7.HK | TIB-124 |
| CD45, mouse | rat/mouse | IgG2a | M1/9.3.4.HL.2 | TIB-122 |
| CD45, mouse | rat/mouse | IgG2a | MB23G2 | HB-220 |
| CD45, mouse | rat/mouse | IgG2a | MB4B4 | HB-223 |
| CD45, pig | mouse | IgM; kappa | 74-9-3 | HB-156 |
| CD45R, mouse | rat/mouse | IgM | RA3-3A1/6.1 | TIB-146 |
| CD45RA, mouse | rat/mouse | IgG2b | 14.8 | TIB-164 |
| CD45RC, mouse | rat/mouse | IgM | I/24.D6 | HB-251 |
| CD47, human | mouse | IgG1 | B6H12.2 | HB-9771 |
| CD49a, human | mouse | IgG1 | TS2/7.1.1 | HB-245 |
| CD49d, sheep | mouse | IgG2b | FW3-218-1 | HB-261 |
| CD54, mouse | rat/mouse | IgG2a | BE29G1 | HB-233 |
| CD57, human | mouse | IgM; kappa | HNK-1 | TIB-200 |
| CD58, human | mouse | IgG1 | TS2/9.1.4.3 | HB-205 |
| CD62E, human | mouse | IgG2a; kappa | CL2 | CRL-2514 |
| CD62E, human | mouse | IgG1; kappa | CL3 | CRL-2515 |
| CD62E, human | mouse | IgG1; kappa | CL37 | CRL-2516 |
| CD62E, human | mouse | IgG2a | H18/7 | HB-11684^{†} |
| CD62L, human | mouse | IgG2a | 1H3 | HB-284 |
| CD62L, human | mouse | IgG1 | DREG200 | HB-302 |
| CD62L, human | mouse | IgG1 | DREG56 | HB-300 |
| CD62L, mouse | rat/mouse | IgG2a | MEL-14 | HB-132 |
| CD62L, sheep and bovine | mouse | IgG1 | DU1-29 | HB-263 |
| CD62P, human | mouse | IgG1 | WAPS 12.2 | HB-299 |
| CD80, mouse | hamster/mouse | IgG | 16-10A1 | HB-301 |
| CD117, human | mouse | IgG2a | BA7.3C.9 | HB-10716^{†} |
| CDw128, human | mouse | IgG2a | 10H2.12.1 | HB-11494^{†} |
| CDw128, human | mouse | IgG2a | 4D1.5.7 | HB-11495^{†} |
| CD151, human | mouse | IgG1 | 41-2 | CRL-2695 |
| CD151, human | mouse | IgG1 | 50-6 | CRL-2696 |
| CD152, mouse | hamster/mouse | IgG | UC10-4F10-11 | HB-304 |
| CD154, human | mouse | IgG2a | 5c8 | HB-10916^{†} |
| CD154, mouse | hamster/mouse | IgG | MR1 | CRL-2580 |
| Cell surface antigen on bovine periodontal | mouse | IgM | PDL-1 | CRL-1882 |
| ligament cells | | | | |
| Cell surface antigen on human myeloma cells | mouse | IgG1 | D 14 | HB-8439^{†} |
| (M-8 antigen system) | | | | |
| Centromere protein B (CENP-B), human | mouse | IgG1 | 2D-7 | HB-9667^{†} |
| Cervical carcinoma, human | human | IgG1; kappa | CLN H11.4 | HB-8307^{†} |
| Cervical carcinoma, human | human | IgM | CLNH5.5 | HB-8206^{†} |
| Channel catfish immunoglobulin | mouse | IgG1; kappa | E-8 | HB-10179^{†} |
| Chlamydia genus-specific antigen | mouse | IgG2b; kappa | 89MS30 | HB-11300^{†} |
| Cholesterol | mouse | IgM | 2C5-6 | HB-8995^{†} |
| Choriocarcinoma tumor cell antigen, human | mouse | IgG2a | K66 | HB-8767^{†} |
| Choriocarcinoma tumor cell antigen, human | mouse | IgG1 | SV63 | HB-8766^{†} |
| Choriocarcinomas, human | mouse | IgG1 (Igh-4a allotype) | 162-46.2 | HB-187 |
| Chronic lymphocytic leukemia (CLL) | mouse | IgG1 | Lym-2 | HB-8613^{†} |
| Class II antigen, beta chain, mouse | hamster/mouse | IgG | KL277 | CRL-2030 |
| Class II antigen, beta chain, mouse | mouse | IgG1 | KL295 | CRL-1996 |
| Class II antigen, beta chain, mouse | mouse | IgG2b | KL304 | CRL-2027 |
| Clathrin, bovine (brain) | mouse | IgM | CVC.4 | TIB-137 |
| Clathrin, heavy chain, human | mouse | IgG1 | TD.1 | CRL-2232 |
| Clathrin, heavy chain, human | mouse | IgG1 | X22 | CRL-2228 |
| Clathrin, light chain, bovine (brain) | mouse | IgG1 | CVC.1 | TIB-135 |
| Clathrin, light chain, bovine (brain) | mouse | IgG2a | CVC.7 | TIB-138 |
| Clathrin, light chain, human | mouse | IgG2b | CON.1 | CRL-2229 |
| Colchicine | mouse | IgG2a | C44 | CRL-1943 |
| Collagen, bone type 1 | mouse | IgG1 | 1H11 | HB-10611^{†} |
| Colon carcinoma-associated antigens (CCAA), | mouse | IgG1; kappa | PCA 31.1 | HB-12314^{†} |
| human | | | | |
| Colon carcinoma-associated antigens (CCAA), | mouse | IgG2a; kappa | PCA 33.28 | HB-12315^{†} |
| human | | | | |
| Colon cells, 29-kDa glycoprotein, human | mouse | IgG2a | HT 29/26 | HB-8247^{†} |
| Colon tumor-associated antigen (CTAA) 16.88 | human | IgG3; kappa | CO 88BV59-1 | CRL-10624^{†} |
| Colon, adenocarcinoma, human | mouse | IgG1 | CLT 85 | HB-8240^{†} |
| Colon, adenocarcinoma, human | mouse | IgG3 | HT 29/36 | HB-8248^{†} |
| Colonic mucin glycoprotein, human | mouse | IgG2a | UC7 | HB-9753^{†} |
| Colonic protein, human | mouse | IgM | 7E12H12 | HB-9397^{†} |
| Colony stimulating factor, subclass I (CSF-I), human | mouse | IgG1 | F18 AF1 | HB-8208^{†} |
| Colony stimulating factor, subclass I (CSF-I), human | mouse | IgG1 | F1A3-23 | HB-8207^{†} |
| Colorectal carcinoma monosialoganglioside | mouse | IgG1 | 1116-NS-19-9 | HB-8059^{†} |
| Colorectal carcinoma-associated tumor antigen | mouse | IgG2b | XMMCO-791 | HB-9173^{†} |
| Common leukocyte antigen, human | mouse | IgG2a | 4B2 | HB-196 |
| Common leukocyte antigen, mouse | rat/mouse | IgG2b | M1/89.18.7.HK | TIB-124 |
| Common leukocyte antigen, mouse | rat/mouse | IgG2a | M1/9.3.4.HL2 | TIB-122 |
| Common leukocyte antigen, mouse | rat/mouse | IgG2a | MB23G2 | HB-220 |
| Common leukocyte antigen, mouse | rat/mouse | IgG2a | MB4B4 | HB-223 |
| Complement C1q, human | mouse | IgG | 12A5B7 | HB-8328^{†} |
| Complement C1q, human | mouse | IgG | 4A4B11 | HB-8327^{†} |
| Complement C3b receptor, human | mouse | IgG1; kappa | Mab 543 | HB-8592^{†} |
| Concanavalin A (Con A) | mouse | IgG1 | 71A7 | TIB-147 |
| Cortical thymic epithelium, mouse | rat/mouse | IgG2a | CDR1 | HB-213 |
| Cortical thymocytes, mouse | rat/mouse | IgM | J11d.2 | TIB-183 |
| Coxsackievirus B4 | mouse | IgG2a; kappa | 204-4 | HB-185 |
| Coxsackievirus B4 | mouse | IgG2a; kappa | 339-1 | HB-186 |
| Coxsackievirus B4 | mouse | IgG2a; kappa | 356-1 | HB-181 |
| Coxsackievirus B4 | mouse | IgG2a; kappa | 38-1 | HB-182 |
| Coxsackievirus-adenovirus receptor (CAR), human | mouse | IgG1 | RmcB | CRL-2379 |
| CR1, human | mouse | IgG1; kappa | Mab 543 | HB-8592^{†} |
| CD3, human | mouse | IgM | 38.1 | HB-231 |
| CD3, human | mouse | IgG2a | OKT 3 | CRL-8001 |
| CD3, mouse | hamster/mouse | IgG | 145-2C11 | CRL-1975 |
| CD4, bovine | mouse | IgG1 | CC30 | HB-270 |
| CD4, bovine | mouse | IgG2a | CC8 | HB-280 |
| CD4, bovine | mouse | IgG2a | IL-A11 | CRL-1879 |
| CD4, human | mouse | IgG2b | OKT 4 | CRL-8002 |
| CD4, mouse | rat/mouse | IgG2b | GK1.5 | TIB-207 |
| CD4, sheep | mouse | IgG1 | 17D | HB-262 |
| CD4a, pig | mouse | IgG2b; kappa | 74-12-4 | HB-147 |
| CD4-binding domain of the gp120 protein of HIV-1 | human/mouse | IgG1 | 448-D | HB-10895^{†} |
| CD4-binding domain of the gp120 protein of HIV-1 | human/mouse | IgG1 | 558-D | HB-10894^{†} |
| CD4-binding domain of the gp120 protein of HIV-1 | human/mouse | IgG1 | 559/64-D | HB-10893^{†} |
| CD5, bovine | mouse | IgG1 | CC17 | HB-281 |
| CD5, bovine | mouse | IgG1 | CC29 | HB-269 |
| CD5, human | mouse | IgG1 | OKT 1 | CRL-8000 |
| CD6, bovine | mouse | IgG2b | CC38 | HB-266 |
| CD6, human | mouse | IgG2a | 12.1 | HB-228 |
| CD6, human | mouse | IgM | 3Pt12B8 | HB-8136^{†} |
| CD7, human | mouse | IgG1; kappa | T3-3A1 | HB-2 |
| CD8, bovine | mouse | IgG1 | CC58 | HB-275 |
| CD8, bovine | mouse | IgG2a | CC63 | HB-264 |
| CD8, bovine | mouse | IgG1 | IL-A51 | CRL-1871 |
| CD8, human | mouse | IgG2a | 51.1 | HB-230 |
| CD8, human | mouse | IgG2a | OKT 8 | CRL-8014 |
| CD8, human | mouse | IgG1 | S6F1 | HB-9579^{†} |
| CD8 alpha 2.2, mouse | mouse | IgM | 83-12-5 | CRL-1971 |
| CD9, mouse | rat/mouse | IgG2a | KMC8.8 | CRL-2212 |
| CD11a, human | mouse | IgG1 | TS2/4.1.1 | HB-244 |
| CD11a, mouse | rat/mouse | IgG2b | FD441.8 | TIB-213 |
| CD11a, mouse | rat/mouse | IgG2a; kappa | M17/4.4.11.9 (new clone | TIB-217 |
| | | | of M17/4.2) | |
| CD11a, mouse | rat/mouse | IgG2b; kappa | M17/5.2 | TIB-237 |
| CD11b, human | mouse | IgM; kappa | 17aba | HB-248 |
| CD11b, human | mouse | IgG2a; kappa | 44aacb | HB-249 |
| CD11b, human | mouse | IgG1 | LM2/1.6.11 | HB-204 |
| CD11b, human | mouse | IgG2b | OKM 1 | CRL-8026 |
| CD11b, mouse | rat/mouse | IgG2b | 5C6 Clone 1 | CRL-1969 |
| CD11c, mouse | hamster/mouse | IgG | N418 | HB-224 |
| CD14, human | mouse | IgG2b; kappa | 26ic | HB-246 |
| CD14, human | mouse | IgG2b | 3C10 | TIB-228 |
| CD14, human | mouse | IgG1; kappa | 60bca | HB-247 |
| CD18, human | mouse | IgG2a; lambda | IB4 | HB-10164^{†} |
| CD18, human | mouse | IgG1 | TS1/18.1.2.11 | HB-203 |
| CD18, mouse | hamster/mouse | IgG | 2E6 | HB-226 |
| CD18, mouse | rat/mouse | IgG2a; kappa | M18/2.a.12.7 (new | TIB-218 |
| | | | clone of M18/2.a.8) | |
| CD19, mouse | rat/mouse | IgG2a; kappa | 1D3 | HB-305 |
| CD20, human | mouse | IgG2a | 1F5 | HB-9645^{†} |
| CD20, human | mouse | IgG1 | C273 | HB-9303^{†} |
| CD21, bovine | mouse | IgG2b | CC51 | HB-271 |
| CD21, human | mouse | IgG2a; kappa | THB-5 | HB-135 |
| CD25, human | mouse | IgG2a | 7G7B6 | HB-8784^{†} |
| CD25, mouse | rat/mouse | IgM; kappa | 7D4 | CRL-1698 |
| CD25, mouse | rat/mouse | IgG1 | PC 61 5.3 | TIB-222 |
| CD28 receptor, mouse | hamster/mouse | IgG | PV1 | HB-12352^{†} |
| CD29, human | mouse | IgG1 | TS2/16.2.1 | HB-243 |
| CD29, mouse | rat/mouse | IgG2a | KMI6 | CRL-2179 |
| CD29, sheep | mouse | IgG1 | FW4-101-1-1 | HB-289 |
| CD32, human | mouse | IgG2b | IV.3 | HB-217 |
| CD32, mouse | rat/mouse | IgG2b | 2.4G2 | HB-197 |
| C-reactive protein, human | mouse | IgG2a; kappa | HD2-4 | HB-86 |
| Creatine kinase - MM and MB, human | rat/mouse | IgG2a; kappa | CKMM 14.15 | HB-9419^{†} |
| Creatine kinase - MM, human | rat/mouse | IgA; kappa | CKMM 14.5 | HB-9420^{†} |
| Creatine kinase - MM, human | rat/mouse | IgG1; kappa | CKMM 14.52 | HB-9421^{†} |
| CTLA-4, mouse | hamster/mouse | IgG | UC10-4F10-11 | HB-304 |
| Cutaneous lymphocyte antigen (CLA), human | rat/mouse | IgM | HECA-452 | HB-11485^{†} |
| Cutaneous melanocytes (M-10 antigen system), | mouse | IgG1 | M 144 | HB-8440^{†} |
| human | | | | |
| Cutaneous melanocytes (M-24 antigen system), | mouse | IgG1 | M-24 (M138) | HB-8449^{†} |
| human | | | | |
| Cutaneous melanocytes (M-25 antigen system), | mouse | IgG2b | L368 | HB-8450^{†} |
| human | | | | |
| Cutaneous melanocytes (M-4 antigen system), | mouse | IgG1 | M 111 | HB-8438^{†} |
| human | | | | |
| Cystic fibrosis transmembrane conductance | mouse | IgG1 | mAb 13-1 | HB-10565^{†} |
| regulator (CFTR) | | | | |
| Cystic fibrosis transmembrane conductance | mouse | IgG2a; kappa | mAB 24-1 | HB-11947^{†} |
| regulator (CFTR) | | | | |
| Cystic fibrosis transmembrane conductance | mouse | IgG1; kappa | mAB 24-2 | HB-11946^{†} |
| regulator (CFTR) | | | | |
| Cytokeratin 18 (CK18) | mouse | IgG | UCD/PR 10.11 | HB-8694^{†} |
| Cytokeratin 8 (CK8) | mouse | IgG | UCD/PR 10.11 | HB-8694^{†} |
| Cytomegalovirus (HCMV) UL18 heavy chain, human | mouse | IgG1 | 10C7 | CRL-2430 |
| Cytomegalovirus (HCMV), immediate —early | mouse | IgG1 | L-14 | HB-8554^{†} |
| antigen, human | | | | |
| Cytomegalovirus (MCMV) m144 heavy chain, | mouse | IgG1 | 15C6 | CRL-2431 |
| mouse | | | | |
| DEC-205, human | mouse | IgG2b | MG38 | CRL-2640 |
| DEC-205, mouse | rat/mouse | IgG2a | DEC-205 | HB-290 |
| Delta heavy chain, human | mouse | IgG3; kappa | δTA4-1 | HB-70 |
| Dendritic cell antigen, human | mouse | IgG2b | MG38 | CRL-2640 |
| Dendritic cell antigen, mouse | rat/mouse | IgG2a | DEC-205 | HB-290 |
| Dendritic cells, mouse | rat/mouse | IgG2b | 33D1 | TIB-227 |
| Dengue virus complex | mouse | IgG2a | D3-2H2-9-21 | HB-114 |
| Dengue virus type 1 | mouse | IgG1 | 15F3-1 | HB-47 |
| Dengue virus type 3 | mouse | IgG1 | 5D4-11 | HB-49 |
| Dengue virus type 4 | mouse | IgG1 | 1H10-6 | HB-48 |
| Dengue virus-2, type specific determinant | mouse | IgG1 | 3H5-1 | HB-46 |
| Dinitrophenyl (DNP) | hamster/mouse | IgG | UC8-1B9 | CRL-1968 |
| Dioxins | mouse | IgG2a; kappa | DD-4 | HB-9743^{†} |
| Diphtheria toxin | human/mouse | IgG | 16M3F10 | HB-8363^{†} |
| Disialosyl Lea (tumor associated fucoganglioside) | mouse | IgG3 | FHCR-1-2516/FH7 | HB-8861^{†} |
| DNA (single stranded) | mouse | IgG3 | MRSS-1 (D₂D₄) | HB-69 |
| DNA polymerase alpha, human | mouse | IgG1 | SJK-132-20 | CRL-1640 |
| DNA polymerase alpha, human | mouse | IgG1 | SJK-237-71 | CRL-1645 |
| DNA polymerase alpha, human | mouse | IgG1 | SJK-287-38 | CRL-1644 |
| DNA polymerase alpha, human | mouse | IgG1 | STK 1 | CRL-1652 |
| DNA polymerase epsilon (pol epsilon), human | mouse | IgG2a | 3C5.1 | CRL-2284 |
| DNA polymerase III holoenzyme, Escherichia coli | mouse | IgM | 123-10 | CRL-1707 |
| DNA polymerase III holoenzyme, Escherichia coli | mouse | IgG1 | 123-28 | CRL-1713 |
| DNA polymerase III holoenzyme, Escherichia coli | mouse | IgM | 68-1-2 | CRL-1712 |
| DNA, double stranded | mouse | IgM | CH26-1352 | HB-8329^{†} |
| DNP and TNP substituted proteins | mouse | IgA; lambda 2 | MOPC 315 | TIB-23 |
| EGF receptor | mouse | IgG1 | 225 | HB-8508^{†} |
| EGF receptor | mouse | IgG1 | 455 | HB-8507^{†} |
| EGF receptor | mouse | IgG2a | 528 | HB-8509^{†} |
| EGF receptor | mouse | IgG | 579 | HB-8506^{†} |
| EGF receptor, human | mouse | IgM | Mab 96 | HB-9763^{†} |
| Eimeria tenella sporozoites | mouse | IgG1 | S1E4 | HB-8332^{†} |
| Eimeria tenella sporozoites | mouse | IgG2a | S3D3 | HB-8331^{†} |
| Eimeria tenella sporozoites and merozoites | mouse | IgG1 | 13.90.2 | HB-8337^{†} |
| Eimeria tenella sporozoites and merozoites | mouse | IgG2a | 2.03.7 | HB-8389^{†} |
| ELAM-1, human | mouse | IgG2a; kappa | CL2 | CRL-2514 |
| ELAM-1, human | mouse | IgG1; kappa | CL3 | CRL-2515 |
| ELAM-1, human | mouse | IgG1; kappa | CL37 | CRL-2516 |
| ELAM-1, human | mouse | IgG2a | H18/7 | HB-11684^{†} |
| EM10 | mouse | IgM; kappa | SM27-1045 | HB-11917^{†} |
| Endothelial cells, IL-1 activated, human | mouse | IgG1 | 7A9 | HB-10135^{†} |
| Endothelial cells, peripheral lymph node, mouse | rat/mouse | IgM | MECA-79 | HB-9479^{†} |
| Endothelial leukocyte adhesion molecule 1 | mouse | IgG2a; kappa | CL2 | CRL-2514 |
| (ELAM-1), human | | | | |
| Endothelial leukocyte adhesion molecule 1 | mouse | IgG1; kappa | CL3 | CRL-2515 |
| (ELAM-1), human | | | | |
| Endothelial leukocyte adhesion molecule 1 | mouse | IgG1; kappa | CL37 | CRL-2516 |
| (ELAM-1), human | | | | |
| Endothelial leukocyte adhesion molecule 1 | mouse | IgG2a | H18/7 | HB-11684^{†} |
| (ELAM-1), human | | | | |
| Endothelium, human | mouse | IgG1; kappa | 10B9 | HB-172 |
| Endothelium, human | mouse | IgG1; kappa | 14E5 | HB-174 |
| Ependymal cell, rat | mouse | IgG2a | RAN-2 | TIB-119 |
| Epidermal growth factor (EGF) receptor | mouse | IgG1 | 225 | HB-8508^{†} |
| Epidermal growth factor (EGF) receptor | mouse | IgG1 | 455 | HB-8507^{†} |
| Epidermal growth factor (EGF) receptor | mouse | IgG2a | 528 | HB-8509^{†} |
| Epidermal growth factor (EGF) receptor | mouse | IgG | 579 | HB-8506^{†} |
| Epidermal growth factor (EGF) receptor, human | mouse | IgM | Mab 96 | HB-9763^{†} |
| Epidermis, basal layer, fetal and neonatal, human | mouse | IgG1 | DAL K20 | CRL-2288 |
| Epidermis, basal layer, fetal and neonatal, human | mouse | IgG1 | DAL K29 | CRL-2291 |
| Epithelial cells, gastrointestinal tract mucosa, | mouse | IgG1 | CLT 152 | HB-8244^{†} |
| 52-kDa protein, human | | | | |
| Epithelium, human | mouse | IgM; kappa | Ep-16 | HB-155 |
| Epstein-Barr virus (EBV) | mouse | IgG1 | 72A1 | HB-168 |
| Epstein-Barr virus (EBV) receptor | mouse | IgG2a; kappa | THB-5 | HB-135 |
| Equine infectious anemia virus (EIAV) core | mouse | IgG1 | EIAV 12E8.1 | HB-8917^{†} |
| antigen (p26) | | | | |
| erb B (v-erb B) oncogene peptide, synthetic | mouse | IgG1 | 171-11B9 | CRL-2661 |
| erb B (v-erb B) oncogene peptide, synthetic | mouse | IgG1 | 172-12A4 | CRL-2660 |
| erb B (v-erb B) oncogene peptide, synthetic | mouse | IgG1; kappa | 173-1C11 | CRL-2659 |
| erb B2 (c-erb B2) protein, human | mouse | IgG1; kappa | 20.3 | CRL-2655 |
| erb B-2 protein, human | mouse | IgG1 | Ab 21.1 | HB-11601^{†} |
| erb B-2 protein, human | mouse | IgG1 | Ab 23.1 | HB-11602^{†} |
| Erythrocytes, mouse | rat/mouse | IgM | J11d.2 | TIB-183 |
| Erythropoietin | mouse | IgG1 | 5F12 AD3 | HB-8209^{†} |
| Erythropoietin, human | rat/mouse | IgG2a | BF-11 | CRL-8164^{†} |
| Escherichia coli 0157:H7 strain 932 | mouse | IgG2a | 4E8C12 | HB-10452^{†} |
| Farnesyltransferase, alpha subunit | mouse | IgG1 | IgG-IB7 | CRL-2418 |
| Fas antigen, human | mouse | IgG1 | huFasM3 | HB-11726^{†} |
| Fas antigen, human | mouse | IgG1 | huFasM38 | HB-11465^{†} |
| Fc alpha receptor, human | mouse | IgM | My 43.51 | HB-12128^{†} |
| Fc gamma receptor, high affinity, human | mouse | IgG1; kappa | CT6-1D7 | CRL-2438 |
| Fc gamma receptor, mouse | rat/mouse | IgG2b | 2.4G2 | HB-197 |
| FcRI, human | mouse | IgG1; kappa | CT6-1D7 | CRL-2438 |
| FcRI, human | mouse | IgM | My 43.51 | HB-12128^{†} |
| FcRII, mouse | rat/mouse | IgG2b | 2.4G2 | HB-197 |
| FcRn heavy chain heterodimers | mouse | IgG1 | 1G3 | CRL-2434 |
| FcRn heavy chain heterodimers | mouse | IgG1 | 2G3 | CRL-2435 |
| Feline leukemia virus (FeLV), p27 protein | mouse | IgG | 24IA₂E₂E₁₀D₅ | HB-8049^{†} |
| Fibrin, human | mouse | | F45J | HB-9740^{†} |
| Fibrin, human | mouse | IgG1; kappa | MH1 | HB-9739^{†} |
| Fibrinogen, human | mouse | | F45J | HB-9740^{†} |
| Fibronectin, human | mouse | IgG1 | HFN 36.3 | CRL-1605 |
| Fibronectin, human | mouse | IgG1 | HFN 7.1 | CRL-1606 |
| Fibronectin, human | mouse | IgG1 | P₃NP/PFn | HB-91 |
| Fibronectin, human, onco-fetal determinant | mouse | IgG1 | FHCR-1-2813/FDC-6 | HB-9018^{†} |
| Fimbriae (2134P) of enterotoxigenic E. coli | mouse | IgG1 | αM346C7C1 | HB-11124^{†} |
| Flavivirus group antigen | mouse | IgG2a | D1-4G2-4-15 | HB-112 |
| Flk-1/KDR | rat/mouse | IgG1; kappa | DC101 | HB-11534^{†} |
| Follicle stimulating hormone (FSH) receptor, human | mouse | IgG1 | FSHR-18 | CRL-2688 |
| Forssman antigen | rat/mouse | IgM | M1/22.25.8.HL | TIB-121 |
| Forssman antigen | rat/mouse | IgM | M1/87.27.7.HLK | TIB-123 |
| fos oncogene peptide, synthetic | mouse | IgG2b; kappa | 411-14E10 | CRL-2663 |
| fos oncogene peptide, synthetic | mouse | IgG1 and IgG2b | 413-15D12 | CRL-2653 |
| Gamma heavy chain, human | mouse | IgG1; kappa | 1410 KG7 | HB-43 |
| Gamma heavy chain, human | mouse | IgG2b; lambda | C3-124 | HB-60 |
| 6B Ganglioside (tumor-associated fucoganglioside) | mouse | IgM | FHCR-1-2624/FH6/ | HB-8873^{†} |
| | | | FHOT-1-3019 | |
| Ganglioside associated with endocrine cells, | mouse | IgM; kappa | 3G5 | CRL-1814 |
| human T lymphocytes, and neuronal cells | | | | |
| Ganglioside GD2 | mouse | IgM | Mab 126 | HB-8568^{†} |
| Gangliosides GD2 and GD3 | mouse | IgG2a | ME361S2a | HB-9326^{†} |
| Gangliosides GM3 and GM4, human | human | IgM; kappa | L612 | CRL-10724^{†} |
| Giardia muris trophozoites | mouse | IgG3; kappa | 1A3.1 | CRL-1961 |
| Giardia muris trophozoites | mouse | IgG2b; kappa | 2B5.3 | CRL-1960 |
| Giardia muris trophozoites | mouse | IgG1; kappa | 3C7.2 | CRL-1959 |
| Glioblastoma, human | mouse | IgM | PI 153/3 | TIB-198 |
| Glomalin (soil glycoprotein) | mouse | IgM; kappa | 32B11 | CRL-2559 |
| Glucocorticoid receptor, mouse and rat | mouse | IgG2b | FIGR | CRL-2173 |
| Glutamic acid decarboxylase (GAD) | mouse | IgG1 | GAD-1 | HB-184 |
| Glycated serum albumin (glycoalbumin) | mouse | IgG | A717 | HB-9596^{†} |
| Glycolipid antigen | mouse | IgM | A2B5 clone 105 | CRL-1520 |
| Glycolipids, di- and trifucosylated type 2 chain | mouse | IgG3 | FHCR-1-2075/FH4 | HB-8775^{†} |
| Glycophorin A, type M | mouse | IgG1; kappa | 6A7M | HB-8159^{†} |
| Glycophorin A, type M and type N | mouse | IgG1; kappa | 10F7MN | HB-8162^{†} |
| Glycophorin A, type N | mouse | IgG1; kappa | 8A2N | HB-8161^{†} |
| Glycophorin A, type N | mouse | IgG2a; kappa | NN-4 | HB-8473^{†} |
| Glycophorin A, type N, human | mouse | IgM; kappa | NN-3 | HB-8474^{†} |
| Glycophorin A, type N, human | mouse | IgG1; kappa | NN-5 | HB-8476^{†} |
| Glycophorin, human | mouse | IgG1 | G26.4.1C3/86 | HB-9893^{†} |
| Glycoprotein antigen, tumor vascular endothelium | mouse | IgG1 | H572 | HB-11608^{†} |
| Glycosphingolipid | mouse | IgM | 1B2-1B7 | TIB-186 |
| Glycosphingolipid | mouse | IgG3 | YI 328-18 | HB-9306^{†} |
| Glycosphingolipid, type II chain H structure | mouse | IgM | BE2 | TIB-182 |
| Golgi complex (GCI), rat | mouse | IgG1 | 6F4C5 | CRL-1869 |
| Golgi vesicular transport protein | mouse | IgM | 4A6 | CRL-1928 |
| Gonadotropin releasing hormone, carboxy terminal | mouse | IgG1 | USASK/DSIL-LHRH-A1 | HB-9094^{†} |
| gp120 glycoprotein | mouse | IgG1 | 55 | HB-9255^{†} |
| gp39, mouse | hamster/mouse | IgG | MR1 | CRL-2580 |
| gp70 envelope antigen (ENV) protein of murine | mouse | IgG2a | 48 | CRL-1913 |
| leukemia viruses (MuLV) | | | | |
| gp70 envelope antigen (ENV) protein of murine | mouse | IgM | 514 | CRL-1914 |
| leukemia viruses (MuLV) | | | | |
| gp90 glycoprotein, human | mouse | IgG2a | G253 | HB-9706^{†} |
| GPIIIa, platelet, human | mouse | IgG1 | AP-3 | HB-242 |
| Granulocyte macrophage colony stimulating | rat/mouse | IgG2a | BVD2-21C113 | HB-9596^{†} |
| factor (GM-CSF), human | | | | |
| Granulocyte macrophage colony stimulating | rat/mouse | IgG2a | BVD2-23B6.4 | HB-9568^{†} |
| factor (GM-CSF), human | | | | |
| Granulocyte, human | mouse | IgG2b | OKM 1 | CRL-8026 |
| Granulocyte, pig | mouse | IgG1; kappa | 74-22-15 | HB-142 |
| Granulocyte, pig | mouse | IgG2b; kappa | 74-22-15A | HB-142.1 |
| Growth hormone (hGH), human | mouse | IgG1; kappa | HGH-B | HB-10596^{†} |
| H-2 (all haplotypes) | rat/mouse | IgG2a | M1/42.3.9.8.HLK | TIB-126 |
| H-2 b | mouse | IgG1; kappa | B8-24-3 | TIB-139 |
| H-2 D b | mouse | IgM; kappa | 23A-5-21S | HB-36 |
| H-2 D b | mouse | IgM; kappa | 28-11-5S | HB-19 |
| H-2 D d | mouse | IgG2a; kappa | 34-2-12S | HB-87 |
| H-2 D d | mouse | IgG2a; kappa | 34-4-20S | HB-75 |
| H-2 D d | mouse | IgM | 34-4-21S | HB-76 |
| H-2 D d | mouse | IgG2a; kappa | 34-5-8S | HB-102 |
| H-2 D k | mouse | IgG2a; kappa | 15-5-5S | HB-24 |
| H-2 from non-k haplotype mice | rat | IgG2a | K204 | HB-221 |
| H-2 K b | mouse | IgM; kappa | 28-13-3S | HB-41 |
| H-2 K b | mouse | IgG2a | AF6-88.5.3 | HB-158 |
| H-2 K b | mouse | IgG2b; kappa | Y-3 | HB-176 |
| H-2 K b, D b | mouse | IgG2a; kappa | 28-8-6S | HB-51 |
| H-2 K d | mouse | IgM | 31-3-4S | HB-77 |
| H-2 K d | mouse | IgG2a | SF1-1.1.10 | HB-159 |
| H-2 K d, D d | mouse | IgG2a; kappa | 34-1-2S | HB-79 |
| H-2 K d, D d | mouse | IgM | 34-7-23S | HB-101 |
| H-2 K k | mouse | IgG2a; kappa | 16-1-11N | HB-16 |
| H-2 K k | mouse | IgG2a; kappa | 16-3-1N | HB-25 |
| H-2 K k | mouse | IgG2a; kappa | 16-3-22S | HB-5 |
| H-2 K k | mouse | IgG1 | AF3-12.1.3 | HB-160 |
| H-2 K k, D k | mouse | IgM; kappa | 12-2-2S (clone 5F11) | HB-50 |
| H-2 K k, D k | mouse | IgG2b; kappa | 15-1-5P | HB-53 |
| H-2 K k, D k | mouse | IgG2a; kappa | 15-3-1S | HB-13 |
| H-2 K k, D k | mouse | IgG2a; kappa | 16-1-2N | HB-14 |
| H-2 K k, D k | mouse | IgG2a; kappa | 3-83P | HB-20 |
| H-2 K of the k, q, p and r haplotypes | mouse | IgG2a | 11-4.1 | TIB-95 |
| H7 flagella, E. coli | mouse | IgG1 | MARC 2B7 | CRL-2509^{†} |
| Hassall's bodies, human | mouse | IgM | TE15 | HB-206 |
| Hassall's bodies, human | mouse | IgG1 | TE16 | HB-210 |
| Hassall's bodies, human | mouse | IgM | TE19 | HB-211 |
| Hassall's bodies, human | mouse | IgG2a | TE8 | HB-212 |
| Heat-stable antigen, mouse | rat/mouse | IgG2b | M1/69.16.11.HL | TIB-125 |
| Heat-stable antigen, mouse | rat/mouse | IgG2c | M1/75.16.4.HLK | TIB-127 |
| HeLA cells | mouse | | 1A₃ | HB-8563^{†} |
| Hematopoietic cells, human | mouse | IgG1 | B3/25 | CRL-8034^{†} |
| Hepatitis B virus surface antigen (HBsAg) | mouse | IgM | H21F8-1 | CRL-8018^{†} |
| Hepatitis B virus surface antigen (HBsAg) | mouse | IgG1 | H25B10 | CRL-8017^{†} |
| Hepatitis B virus surface antigen (HBsAg) | mouse | IgG1 | H25B10 | CRL-8017A^{†} |
| HER2 receptor | mouse | IgG1 | A-HER2 | CRL-10463^{†} |
| Herpes simplex virus type 1 (HSV-1) glycoprotein | mouse | IgG2a | 52-S | HB-8181^{†} |
| Herpes simplex virus type 1 (HSV-1) glycoprotein | mouse | IgG2a | 53-S | HB-8182^{†} |
| Herpes simplex virus type 1 (HSV-1), immediate | mouse | IgG2a | 58-S | HB-8183^{†} |
| early protein (ICP 4) | | | | |
| Herpes simplex virus type 1 (HSV-1), internal capsid | mouse | IgG2a | 39-S | HB-8180^{†} |
| protein 8 (ICP 8) | | | | |
| Herpes simplex virus type 1 (HSV-1), nucleocapsid | mouse | IgG1 | 1D4 | HB-8068^{†} |
| protein (p40) | | | | |
| Herpes simplex virus type 2 (HSV-2), nucleocapsid | mouse | IgG1 | 3E1 | HB-8067^{†} |
| protein (p40) | | | | |
| HFE | mouse | IgG1 | 1C3 | CRL-2441 |
| HFE | mouse | IgG1 | 2A11 | CRL-2442 |
| HFE | mouse | IgM; kappa | 2A5 | CRL-2444 |
| HFE | mouse | IgG1 | 2B7 | CRL-2443 |
| HFE | mouse | IgM; kappa | 3A5 | CRL-2440 |
| HIV gp41 | mouse | IgG1; kappa | MH-SVM25 | HB-8871^{†} |
| HIV p17 | mouse | IgG1; kappa | MH-SVM33C9 | HB-8975^{†} |
| HIV p24 | mouse | IgG2; kappa | MH-SVM23 | HB-8870^{†} |
| HIV p24 | mouse | IgG1; kappa | MH-SVM26 | HB-8872^{†} |
| HIV-1 gp120 | mouse | IgG1 | 46-2 | CRL-2186 |
| HIV-1 gp120 | mouse | IgG1 | 46-4 | CRL-2178 |
| HIV-1 gp120 | mouse | IgG1 | 46-5 | CRL-2184 |
| HIV-1 gp120 | mouse | IgG1 | 55-2 | CRL-2155 |
| HIV-1 gp120 | mouse | IgG1 | 55-36 | CRL-2153 |
| HIV-1 gp120 | mouse | IgG2a | 55-6 | CRL-2156 |
| HIV-1 gp120 | mouse | IgG2a | 55-83 | CRL-2185 |
| HIV-1 gp120 | mouse | IgG1 | 803-15.6 | CRL-2395 |
| HIV-1 p17 | mouse | IgG1; kappa | MH-SVM33C9 | HB-8975^{†} |
| HIV-1 p24 | mouse | IgG1 | 31-42-19 | HB-9726^{†} |
| HIV-1 p24 | mouse | IgG1 | 31-90-25 | HB-9725^{†} |
| HLA A2, B17 | mouse | IgG1 | MA2.1 | HB-54 |
| HLA A3 | mouse | IgG2a; kappa | GAP A3 | HB-122 |
| HLA A3 | rat/mouse | IgM | WFL3C6.1 | HB-8157^{†} |
| HLA B27, B7 | mouse | IgG2a | B27M1 | HB-157 |
| HLA B27, Bw47 | mouse | IgM | B27M2 | HB-165 |
| HLA B5 | mouse | IgG1 | 4D12 | HB-178 |
| HLA B7 | mouse | IgG1 | BB7.1 | HB-56 |
| HLA B7, B40 | mouse | IgG1 | MB 40.3 | HB-105 |
| HLA B7, B40 | mouse | IgG1 | MB40.2 | HB-59 |
| HLA B7, Bw22, B27 | mouse | IgG1 | ME 1 | HB-119 |
| HLA B7, B40 | mouse | IgG1 | BB7.6 | HB-115 |
| HLA Bw6 | rat/mouse | IgG2b | SFR8-B6 | HB-152 |
| HLA DC1 | mouse | IgG2a | G2a.5 | HB-110 |
| HLA DC1 | mouse | IgG2b | G2b.2 | HB-109 |
| HLA DQ | mouse | IgG1; kappa | IVD12 | HB-144 |
| HLA DQw1 | mouse | IgG1 | Genox 3.53 | HB-103 |
| HLA DR | mouse | IgG1 | Antibody 2.06 | HB-104 |
| HLA DR, DP, DQ | mouse | IgG1; kappa | IVA12 | HB-145 |
| HLA DR, DQ | mouse | IgG2a | 9.3F10 | HB-180 |
| HLA DR5 | rat/mouse | IgG2b | SFR3-DR5 | HB-151 |
| HLA heavy chain | mouse | IgG2a; kappa | 171-4 | HB-296 |
| HLA-DR alpha chain | mouse | IgG2a | LB3.1 | HB-298 |
| HMG-CoA reductase (3-hydroxy-3-methyl-glutaryl | mouse | IgG1 | A9 | CRL-1811 |
| Coenzyme A reductase) | | | | |
| Hog renin | mouse | IgG1 | F32 VIII C4 | CRL-1653 |
| HTLV-III gp41 | mouse | IgG1; kappa | MH-SVM25 | HB-8871^{†} |
| HTLV-III p17 | mouse | IgG1; kappa | MH-SVM33C9 | HB-8975^{†} |
| HTLV-III p24 | mouse | IgG2; kappa | MH-SVM23 | HB-8870^{†} |
| HTLV-III p24 | mouse | IgG1; kappa | MH-SVM26 | HB-8872^{†} |
| Human epidermal growth factor (EGF) receptor | mouse | IgG2a; kappa | Mab 108 | HB-9764^{†} |
| H-Y antigen | mouse | IgM; kappa | 12/44 | HB-9070^{†} |
| H-Y antigen | mouse | IgM; kappa | 12/49 | HB-9071^{†} |
| I-A | mouse | IgG2a | Y-3P | HB-183 |
| I-A b | mouse | IgM | 25-5-16S | HB-37 |
| I-A b | mouse | IgM | 25-9-3S | HB-38 |
| I-A b | mouse | IgG2a | AF6-120-1.2 | HB-163 |
| I-A b and I-A d | mouse | IgG2a; kappa | 25-9-17S II | HB-26 |
| I-A b and I-A d | mouse | IgG3 | BP107.2.2 | TIB-154 |
| I-A b, I-A d | mouse | IgM; kappa | 28-16-8S | HB-35 |
| I-A b, I-A d, I-A q, I-E d and I-E k | rat/mouse | IgG2b; kappa | M5/114.15.2 | TIB-120 |
| I-A b, d | rat/mouse | IgG2b | B21-2 | TIB-229 |
| I-A d | mouse | IgG2a; kappa | 34-5-3S | HB-85 |
| I-A d | mouse | IgG2a; kappa | MK-D6 | HB-3 |
| I-A k | mouse | IgG2b | 11-5.2.1.9 | TIB-94 |
| I-A k | mouse | IgM; kappa | 26-7-11S | HB-15 |
| I-A k | mouse | IgM | 26-8-16S | HB-42 |
| I-A of k, r, f and s haplotypes | mouse | IgG2a | 10-3.6.2 | TIB-92 |
| I-A of the k, r, f and s haplotypes | mouse | IgG2b | 10-2.16 | TIB-93 |
| I-A s | mouse | IgG2b | MK-S4 | HB-4 |
| Ia, human | mouse | IgG1; kappa | L203 | HB-171 |
| Ia, human | mouse | IgG1; kappa | L227 | HB-96 |
| Ia, human | mouse | IgG2a | L243 | HB-55 |
| I-A, I-E, monomorphic, mouse | hamster/mouse | IgG | N22 | HB-225 |
| Ia, rabbit | mouse | IgG2a | 2C4 | CRL-1760 |
| ICAM-1, canine | mouse | IgG1 | CL18/6 | CRL-2518 |
| ICAM-1, human | mouse | IgG2a | R6.5.D6.E9.B2 | HB-9580^{†} |
| ICAM-1, mouse | rat | IgG2b | YN1/1.7.4 | CRL-1878 |
| ICAM-1, mouse | rat/mouse | IgG2a | BE29G1 | HB-233 |
| ICAM-4, rat | mouse | IgG1 | 127H | HB-11911^{†} |
| Idiotypic determinant on on anti-chlamydia genus | mouse | IgG1; kappa | 91MS441 | HB-11301^{†} |
| antibody | | | | |
| Idiotypic determinant on the P3X63Ag8 myeloma | mouse protein | IgG2b; kappa | 80 V 5B4 | TIB-132 |
| I-E | mouse | IgG2b | Y-17 | HB-179 |
| I-E k | mouse | IgG2a; kappa | 14-4-4S | HB-32 |
| I-E k | mouse | IgG2a; kappa | 17-3-3S | HB-6 |
| Ig-4a allotype on mouse IgG1 | mouse | IgG2a | Ig(4a)10.9 | HB-146 |
| IgA, human | mouse | IgG1; kappa | CH-EB6 | HB-200 |
| IgD, Ig-5a allotype, mouse | mouse | IgG2a | Ig(5a)7.2 (formerly 10- | TIB-149 |
| | | | 4-22) | |
| IgD, mouse | rat/mouse | IgG2a | 11-26c | HB-250 |
| IgE, human | mouse | IgG1; kappa | CIA-E-4.15 | HB-235 |
| IgE, human | mouse | IgG1; kappa | CIA-E-7.12 | HB-236 |
| IgE, human | mouse | IgG2a; kappa | E5BB3IIA2 | HB-121 |
| IgG (Fc), human | mouse | IgG2b | HP6000 | CRL-1754 |
| IgG (Fc), human | mouse | IgG2a | HP6017 | CRL-1753 |
| IgG (Fc), human | mouse | IgG1 | HP605B | CRL-1786 |
| IgG (Fd, F(ab′)2, Fab), human | mouse | IgG2a | HP6045 | CRL-1757 |
| IgG Fc receptor, human | mouse | IgG2b | IV.3 | HB-217 |
| IgG1 (Fc), human | mouse | IgG2b | HP6001 | CRL-1755 |
| IgG1 (Fc), rat | mouse | IgG2b | RG11/39.4 | TIB-170 |
| IgG2 (Fc), human | mouse | IgG1 | HP6002 | CRL-1788 |
| IgG2 (Fd), human | mouse | IgG1 | HP6014 | CRL-1752 |
| IgG2, mouse | rat/mouse | IgG2b; kappa | 7D2-1.4.1.5 | HB-92 |
| IgG2, mouse | rat/mouse | IgG2a | ED1-19-1-6-5 | HB-90 |
| IgG2a (Fab′), rat | mouse | IgG2b | RG9/6.13 HLK | TIB-167 |
| IgG2a (Fc), rat | mouse | IgG2b | RG7/1.30 | TIB-173 |
| IgG2a Ig-1a allotype, mouse | mouse | IgG2a | Ig(1a)8.3 (formerly 20-8.3) | TIB-148 |
| IgG2a, Igh-1b allotype, mouse | mouse | IgM | RDP 45/20 | TIB-98 |
| IgG2b (Fc), rat | mouse | IgG2b | RG7/11.1 | TIB-174 |
| IgG3 (Fc), human | mouse | IgG1 | HP6003 | CRL-1756 |
| IgG3 (hinge), human | mouse | IgG1 | HP6047 | CRL-1774 |
| IgG3 hinge region, human | mouse | IgG1 | HP6050 | CRL-1768 |
| IgG3, mouse | rat/mouse | IgG1 | 2E.6 | HB-128 |
| IgG4 (Fab), human | mouse | IgG2a | HP6020 | CRL-1789 |
| IgG4 (Fc), human | mouse | IgG3 | HP6023 | CRL-1776 |
| IgG4 (Fc), human | mouse | IgG1 | HP6025 | CRL-1775 |
| Igh-5.3 (IgD b, e) | mouse | IgG1 | AF6-122.2.5 | HB-199 |
| Igh-5.4 (IgD a) | mouse | IgG2b | AMS 9.1.1.1 | HB-161 |
| Igh-5.5 (IgD e) | mouse | IgG2b | AF4-73.3.1 | HB-201 |
| Igh-5b (IgD b allotype) | mouse | IgG1 | Ig(5b)6.3 | TIB-96 |
| Igh-6.6 (IgM b) | mouse | IgG1 | AF6-78.25.4 | HB-162 |
| IgM (Igh-6.5 allotype), mouse | rat/mouse | IgG1; kappa | Bet-1 | HB-100 |
| IgM (lambda), human | mouse | IgG2b; kappa | LP4.4 | HB-232 |
| IgM (mu heavy chain), mouse | rat/mouse | IgG2b | 331.12 | TIB-129 |
| IgM, bovine | mouse | IgG1 | IL-A30 | CRL-1894 |
| IgM, human | mouse | IgG1 | M-2E6 | HB-138 |
| IgM, mouse | rat/mouse | IgG1; kappa | Bet-2 | HB-88 |
| IgM, pig | mouse | IgG1; kappa | 5C9 | HB-8371^{†} |
| IgM, rabbit | mouse | IgG1 | NRbM | CRL-1839 |
| IL-1 beta, human | mouse | IgG1; kappa | ILB1-H21 | HB-10220^{†} |
| IL-1 beta, human | mouse | IgG1; kappa | ILB1-H34 | HB-10221^{†} |
| IL-1 beta, human | mouse | IgG1; kappa | ILB1-H6 | HB-10219^{†} |
| IL-1 beta, human | mouse | IgG2b; kappa | ILB1-H67 | HB-10222^{†} |
| IL-1 beta, recombinant, bovine | mouse | IgG1 | SA22 | CRL-2052 |
| IL-2, mouse | rat/mouse | IgG2a | S4B6-1 | HB-10968^{†} |
| IL-4, mouse | rat/mouse | IgG1 | 11B11 | HB-188 |
| IL-8, human | mouse | IgG1 | EL-NC-1S | HB-9647^{†} |
| IL-12 p40, mouse | rat/mouse | IgG2a | R1-5D9 | CRL-2360 |
| IL-12 p40, mouse | rat/mouse | IgG2b | R2-10F6 | CRL-2358 |
| IL-12 p75, mouse | rat/mouse | IgG2b | R2-9A5 | CRL-2357 |
| IL-12, human | rat/mouse | IgG1 | 20C2 | CRL-2382 |
| IL-15, human | mouse | IgG1 | hIL-15-M110 | HB-12061^{†} |
| IL-15, human | mouse | IgG1 | hIL-15-M111 | HB-12062^{†} |
| Infectious bovine rhinotracheitis virus (IBRV) | mouse | IgG1 | 1B8-F11 | CRL-1852 |
| Infectious bursal disease (IBD) virus | mouse | IgG2a; kappa | B69 | HB-9437^{†} |
| Infectious bursal disease (IBD) virus | mouse | IgG2a; kappa | R63 | HB-9490^{†} |
| Infectious bursal disease (IBD) virus, serotypes | mouse | IgG1; kappa | B29 | HB-9746^{†} |
| 1 and 2 | | | | |
| Inflammatory cells, human | mouse | IgG1 | MY904 | HB-9510^{†} |
| Influenzavirus A matrix protein (M) | mouse | IgG1 | M2-1C6-4R3 | HB-64 |
| Influenzavirus A nucleoprotein | mouse | IgG1 | 46/4 | HB-67 |
| Influenzavirus A nucleoprotein | mouse | IgG2a | H16-L10-4RS | HB-65 |
| Influenzavirus hemagglutinin | mouse | IgG2a | 73/1 | HB-66 |
| Insulin | mouse | IgG2a; kappa | BE3F9 | HB-133 |
| Insulin | mouse | IgG1; kappa | CC9C10 | HB-123 |
| Insulin | mouse | IgG1; kappa | CE9H9 | HB-127 |
| Insulin | mouse | IgG1; kappa | CG7C7 | HB-126 |
| Insulin | mouse | IgG2a; kappa | DB9G8 | HB-124 |
| Insulin (residues A8-10), human | mouse | IgG1; kappa | AE9D6 | HB-125 |
| Insulin receptor, human | mouse | IgG1; kappa | αIR-1 | HB-175 |
| Insulin receptor, placental, human | mouse | IgG1 | DII 33.1 | CRL-1827 |
| Integrin, alpha 1, human | mouse | IgG1 | TS2/7.1.1 | HB-245 |
| Integrin, alpha 4, sheep | mouse | IgG2b | FW3-218-1 | HB-261 |
| Integrin, alpha 4/beta 7, mouse | rat/mouse | IgG2a | DATK32 | HB-294 |
| Integrin, beta 1 subunit, mouse | rat/mouse | IgG2a | KMI6 | CRL-2179 |
| Integrin, beta 1, human | mouse | IgG1 | TS2/16.2.1 | NB-243 |
| Integrin, beta 1, sheep | mouse | IgG1 | FW4-101-1-1 | HB-289 |
| Integrin, beta 2, mouse | hamster/mouse | IgG | 2E6 | HB-226 |
| Integrin, beta 3, human | mouse | IgG1 | AP-3 | HB-242 |
| Integrin, beta 7 Integrin, mouse | rat/mouse | IgG2a | FIB21 | HB-295 |
| Integrin, beta 7 Integrin, mouse | rat/mouse | IgG2a | FIB504.64 | HB-293 |
| Integrin, leukocyte, mouse | hamster/mouse | IgG | N418 | HB-224 |
| Integrin-associated protein, human | mouse | IgG1 | B6H12.2 | HB-9771^{†} |
| Integrin-like cellular adhesion molecule | rat/mouse | IgG2b | PS/2 | CRL-1911 |
| Intercellular adhesion molecule 1, mouse | rat | IgG2b | YN1/1.7.4 | CRL-1878 |
| Intercellular adhesion molecule 1, mouse | rat/mouse | IgG2a | BE29G1 | HB-233 |
| Interferon gamma receptor, mouse | rat/mouse | IgA; kappa | GR-96 | CRL-2013 |
| Interferon gamma receptor, mouse | rat/mouse | IgG2a; kappa | GR-20 | CRL-2024 |
| Interferon, gamma, human | mouse | IgG1 | γ3-11.1 | HB-8700^{†} |
| Interferon, gamma, human | mouse | IgG1 | IFGCP-F1BA10 | HB-8291^{†} |
| Interferon, gamma, mouse | rat/mouse | IgG1 | R4-6A2 | HB-170 |
| Interleukin 1 beta, human | mouse | IgG1; kappa | ILB1-H21 | HB-10220^{†} |
| Interleukin 1 beta, human | mouse | IgG1; kappa | ILB1-H34 | HB-10221^{†} |
| Interleukin 1 beta, human | mouse | IgG1; kappa | ILB1-H6 | HB-10219^{†} |
| Interleukin 1 beta, human | mouse | IgG2b; kappa | ILB1-H67 | HB-10222^{†} |
| Interleukin 1 beta, recombinant, bovine | mouse | IgG1 | SA22 | CRL-2052 |
| Interleukin 15, human | mouse | IgG1 | hIL-15-M110 | HB-12061^{†} |
| Interleukin 15, human | mouse | IgG1 | hIL-15-M111 | HB-12062^{†} |
| Interleukin 2 receptor, human | mouse | IgG1 | 2A3A1H | HB-8555^{†} |
| Interleukin 2 receptor, human | mouse | IgG2a | 7G7B6 | HB-8784^{†} |
| Interleukin 2 receptor, mouse | rat/mouse | IgM; kappa | 7D4 | CRL-1698 |
| Interleukin 2 receptor, mouse | rat/mouse | IgG1 | PC 61 5.3 | TIB-222 |
| Interleukin 2, mouse | rat/mouse | IgG2a | S4B6-1 | HB-10968^{†} |
| Interleukin 8 receptor type B (IL-8R-B), human | mouse | IgG2a | 10H2.12.1 | HB-11494^{†} |
| Interleukin 8 receptor type B (IL-8R-B), human | mouse | IgG2a | 4D1.5.7 | HB-11495^{†} |
| Interleukin 8, human | mouse | IgG1 | EL-NC-1S | HB-9647^{†} |
| Intermediate filaments | mouse | IgG1 | α Intermediate Filament | TIB-131 |
| Intracellular adhesion molecule 1 (ICAM-1), canine | mouse | IgG1 | CL18/6 | CRL-2518 |
| Intracellular adhesion molecule 1 (ICAM-1), human | mouse | IgG2a | R6.5.D6.E9.B2 | HB-9580^{†} |
| Intracellular adhesion molecule 1 (ICAM-1), mouse | rat | IgG2b | YN1/1.7.4 | CRL-1878 |
| Intracellular adhesion molecule 1 (ICAM-1), mouse | rat/mouse | IgG2a | BE29G1 | HB-233 |
| Intracellular adhesion molecule 1 (ICAM-1), rat | mouse | IgG1 | 127H | HB-11911^{†} |
| Invected protein, Drosophila melanogaster | mouse | IgG1 | 4D9D4 | CRL-1818 |
| J5 endotoxin core, Escherichia coli | mouse | IgG1 | J5-1 | HB-8297^{†} |
| J5 endotoxin core, Escherichia coli | mouse | IgG1 | J5-2 | HB-8298^{†} |
| K99 pilus, Escherichia coli | mouse | IgG1; kappa | 2BD4E4 K99 | HB-8178^{†} |
| Kappa light chain (monotypic determinant), rat | mouse | IgG2b | RG7/9.1 HLK | TIB-169 |
| Kappa light chain (RI-1a and RI-1b allotypes), rat | mouse | IgG2a; kappa | MAR 18.5 | TIB-216 |
| Kappa light chain [kappa 1b (LEW)], rat | mouse | IgG2a | RG7/7.6 HL | TIB-172 |
| Kappa light chain, human | mouse | IgG1; kappa | 141PF11 | HB-45 |
| Kappa light chain, human | mouse | IgG1 | HP6053 | CRL-1758 |
| Kappa light chain, human | mouse | IgG1; kappa | TB 28-2 | HB-61 |
| Kappa light chain, mouse | rat/mouse | IgG1 | 187.1 | HB-58 |
| Kidney tubules, human | mouse | IgG1 | DAL K20 | CRL-2288 |
| Kidney tubules, human | mouse | IgG1 | DAL K29 | CRL-2291 |
| Kininogen heavy chain, human | mouse | IgG1 | 2B5 | HB-8963^{†} |
| Kininogen light chain, human | mouse | IgG1 | C11C1 | HB-8964^{†} |
| Kunitz soybean trypsin inhibitor | mouse | IgG1; kappa | C129 | HB-9516^{†} |
| Kunitz soybean trypsin inhibitor | mouse | IgG1; kappa | C171 | HB-9515^{†} |
| Kunitz soybean trypsin inhibitor | mouse | IgG1; kappa | C180 | HB-9517^{†} |
| L d, D b and D q | mouse | IgG2a; kappa | 28-14-8S | HB-27 |
| L d, D q, L q and L b | mouse | IgG2a; kappa | 30-5-7S | HB-31 |
| L3T4 antigen (T cell), mouse | rat/mouse | IgG2b | GK1.5 | TIB-207 |
| La Crosse Virus, G1 envelope glycoprotein | mouse | IgG2b | 807.15 | CRL-2287 |
| La Crosse Virus, G1 envelope glycoprotein | mouse | IgG1 | 807.31 | CRL-2282 |
| La Crosse Virus, G1 envelope glycoprotein | mouse | IgG2a | 807.33 | CRL-2290 |
| La/SSB, bovine | mouse | IgG | La1 | HB-8609^{†} |
| LAM-1, human | mouse | IgG1 | DREG200 | HB-302 |
| LAM-1, human | mouse | IgG1 | DREG56 | HB-300 |
| Lambda 1 light chain, mouse | mouse | IgG1; kappa | LS-136 | TIB-157 |
| Lambda light chain, human | mouse | IgG2a | HP6054 | CRL-1763 |
| Laminin | rat/mouse | IgG2b | 2AB1-IA10 | HB-8210^{†} |
| LECAM, human | mouse | IgG1 | DREG200 | HB-302 |
| LECAM, human | mouse | IgG1 | DREG56 | HB-300 |
| Legionella pneumophila | mouse | IgG2a | LP3IIG2 | HB-8472^{†} |
| Legionella pneumophila serogroup 1 | mouse | IgG3 | Lp1 MAB 1 | CRL-1765 |
| Legionella pneumophila serogroup 1 | mouse | IgG2b | Lp1 MAB 2 | CRL-1770 |
| Legionella pneumophila serogroup 1 | mouse | IgG2b | Lp1 MAB 3 | CRL-1767 |
| Leptomeningeal cell, rat neural antigen-2, RAN-2) | mouse | IgG2a | Ran-2 | TIB-119 |
| Leptospira pomona type kennewicki | mouse | IgA | 2D7F10 | CRL-2025 |
| Leu 200 glycoproteins, human | mouse | IgG2a; kappa | 4C | HB-8311^{†} |
| Leu-5 | mouse | IgM | TM1 | HB-169 |
| Leu8, human | mouse | IgG1 | DREG200 | HB-302 |
| Leu8, human | mouse | IgG1 | DREG56 | HB-300 |
| Leu8, mouse | rat/mouse | IgG2a | MEL-14 | HB-132 |
| Leukocyte common antigen, human | mouse | IgG2a; kappa | GAP 8.3 | HB-12 |
| Leukocyte function antigen 1, alpha subunit, mouse | rat/mouse | IgG2a; kappa | M17/4.4.11.9 (new | TIB-217 |
| | | | clone of M17/4.2) | |
| Leukocyte function antigen 1, mouse | rat/mouse | IgG2b | FD441.8 | TIB-213 |
| LEW RT1.A | rat/mouse | IgM | WFL3C6.1 | HB-8157^{†} |
| LEW RT1.A | rat/mouse | IgM | WFL4F12.3 | HB-8156^{†} |
| Lewis a and b blood group antigens, human | mouse | IgG3 | 151-5-G2-12 | HB-8322^{†} |
| Lewis a and b blood group antigens, human | mouse | IgG3 | 151-5-G3-5 | HB-8323^{†} |
| Lewis a antigen | mouse | IgG1 | BC9-E5 | CRL-1670 |
| Lewis a antigen | mouse | IgG2a; kappa | CA3-F4 | CRL-1667 |
| Lewis a antigen | mouse | IgG1; kappa | CF4-C4 | CRL-1716 |
| Lewis a blood group antigen, human | mouse | IgG3 | 151-6-A7-9 | HB-8324^{†} |
| Lewis b blood group antigen, human | mouse | IgG1 | 130-3-F7-5 | HB-8326^{†} |
| Lewis b blood group antigen, human | mouse | IgM | 143-2-A6-11 | HB-8325^{†} |
| Lex (tumor-associated fucoganglioside) | mouse | IgM | FHCR-1-2624/FH6/ | HB-8873^{†} |
| | | | FHOT-1-3019 | |
| LFA-1, beta subunit, mouse | rat/mouse | IgG2a; kappa | M18/2.a.12.7 (new | TIB-218 |
| | | | clone of M18/2.a.8) | |
| LFA-1, mouse | rat/mouse | IgG2b | FD441.8 | TIB-213 |
| LFA-1, mouse | rat/mouse | IgG2a; kappa | M17/4.4.11.9 (new | TIB-217 |
| | | | clone of M17/4.2) | |
| LFA-1, mouse | rat/mouse | IgG2b; kappa | M17/5.2 | TIB-237 |
| LGL-1 | rat/mouse | IgG2a | 4D11 | HB-240 |
| Lgp100a | rat/mouse | IgG2a | 30-C7 | TIB-106 |
| Lipopolysaccharide, chlamydia | mouse | IgG3 | L2I-6 | HB-8705^{†} |
| Lipoprotein H2, Pseudomonas aeruginosa | mouse | IgG1 | MA1-6 | CRL-1783 |
| Lipoprotein receptor related protein (LRP), | mouse | IgG1 | IgG-5D7 | CRL-1938 |
| 515-kDa subunit, rabbit | | | | |
| Lipoprotein receptor related protein (LRP), | mouse | IgG1 | IgG-1B3 | CRL-1937 |
| 85-kDa subunit, rabbit | | | | |
| Lipoprotein receptor related protein (LRP), carboxy | mouse | IgG1 | IgG-11H4 | CRL-1936 |
| terminal | | | | |
| Low density lipoprotein (LDL) receptor, bovine | mouse | IgG1 | 9D9 | CRL-1703 |
| Low density lipoprotein (LDL) receptor, bovine | mouse | IgG2b | C7 | CRL-1691 |
| Low density lipoprotein (LDL) receptor, human | mouse | IgG1 | IgG-4A4 | CRL-1898 |
| Low density lipoprotein (LDL), human | mouse | IgG1 | B1B3 | CRL-2249 |
| Low density lipoprotein (LDL), human | mouse | IgG1 | B1B6 | CRL-2248 |
| LPAM-1, mouse | rat/mouse | IgG2b | R1-2 | HB-227 |
| Lung cancer | mouse | IgG1 | L18 | HB-8628^{†} |
| Lung cancer | mouse | IgM | L5 | HB-8627^{†} |
| Lung cancer, human | mouse | IgG2a; kappa | 703D4 | HB-8301^{†} |
| Luteinizing hormone releasing hormone (LHRH), | mouse | IgG1 | USASK/DSIL-LHRH-A1 | HB-9094^{†} |
| carboxy terminal | | | | |
| Luteinizing hormone/chorionic gonadotropin | mouse | IgG2a | FSHR-323 | CRL-2689 |
| (LH/hCG) receptor, human | | | | |
| Luteinizing hormone/chorionic gonadotropin | mouse | IgG1 | LHR-1055 | CRL-2687 |
| (LH/hCG) receptor, human | | | | |
| Luteinizing hormone/chorionic gonadotropin | mouse | IgG1 | LHR-29 | CRL-2685 |
| (LH/hCG) receptor, human | | | | |
| Luteinizing hormone/chorionic gonadotropin | mouse | IgG1 | LHR-74 | CRL-2686 |
| (LH/hCG) receptor, human | | | | |
| Ly 6.2C, mouse | mouse | IgG1 | 143-4.2 | CRL-1970 |
| Lyb 2.1, mouse | mouse | IgG2b | 10-1.D.2 | TIB-165 |
| Lyb 8.2, mouse | mouse | IgG1 | Cy34.1.2 | TIB-163 |
| Lymphocyte function antigen 1 (LFA-1) alpha | mouse | IgG1 | TS1/22.1.1.13 | HB-202 |
| subunit, human | | | | |
| Lymphocyte function antigen 1 (LFA-1) beta | mouse | IgG1 | TS1/18.1.2.11 | HB-203 |
| subunit, human | | | | |
| Lymphocyte function antigen 1 (LFA-1), human | mouse | IgG1 | TS2/4.1.1 | HB-244 |
| Lymphocyte function antigen 1, mouse | rat/mouse | IgG2b; kappa | M17/5.2 | TIB-237 |
| Lymphocyte function antigen 2 (LFA-2), human | mouse | IgG1 | TS2/18.1.1 | HB-195 |
| Lymphocyte function antigen 3 (LFA-3), human | mouse | IgG1 | TS2/9.1.4.3 | HB-205 |
| Lymphocyte Peyer's patch HEV adhesion molecule, | rat/mouse | IgG2b | R1-2 | HB-227 |
| mouse | | | | |
| Lymphocyte surface receptor for endothelium, | rat/mouse | IgG2a | MEL-14 | HB-132 |
| mouse | | | | |
| Lymphocyte, mouse | rat/mouse | IgM | GL7 | HB-254 |
| Lymphoma cells, canine | mouse | IgG2a | Hybridoma 231 | HB-9401^{†} |
| Lymphoma cells, canine | mouse | IgG1 | Hybridoma 234 | HB-9402^{†} |
| Lymphoma cells, canine | mouse | IgG2a | Hybridoma 234 s.2a | HB-9403^{†} |
| Lyt 2.2, mouse | mouse | IgM | 83-12-5 | CRL-1971 |
| Lyt-1 (all alleles), mouse | rat/mouse | IgG2a | 53-7.313 | TIB-104 |
| Lyt-2 (all alleles), mouse | rat/mouse | IgM | 3.15S | TIB-211 |
| Lyt-2 (all alleles), mouse | rat/mouse | IgG2a | 53-6.72 | TIB-105 |
| Lyt-2.1, mouse | mouse | IgG2a | 116-13.1 | HB-129 |
| Lyt-2.2, mouse | mouse | IgM | 41-3.48 | HB-130 |
| Lyt-2.2, mouse | mouse | IgM | HO-2.2 | TIB-150 |
| Lyt-2.2, mouse | rat/mouse | IgG2b | 2.43 | TIB-210 |
| Mac-1, alpha chain, mouse | rat/mouse | IgG2b | M1/70.15.11.5.HL | TIB-128 |
| Mac-1, beta subunit, mouse | rat/mouse | IgG2a; kappa | M18/2.a.12.7 (new | TIB-218 |
| | | | clone of M18/2.a.8) | |
| Mac-1, human | mouse | IgG1 | LM2/1.6.11 | HB-204 |
| Mac-2, mouse | rat/mouse | IgG2a; kappa | M3/38.1.2.8 HL2 | TIB-166 |
| Mac-3 (mouse macrophage antigen, 110-kDa | rat/mouse | IgG1; kappa | M3/84.6.34 | TIB-168 |
| glycoprotein) | | | | |
| Macrophage, activated, mouse | rat/mouse | IgG2a | 158.2 | HB-8466^{†} |
| Macrophage, human | mouse | IgG1; kappa | 14E5 | HB-174 |
| Macrophage, mouse | rat/mouse | IgG2b | F4/80 | HB-198 |
| Macrophage, mouse | rat/mouse | IgG2b | M1/70.15.11.5.HL | TIB-128 |
| Macrophage, mouse | rat/mouse | IgG2a; kappa | M3/38.1.2.8 HL2 | TIB-166 |
| Macrophage, pig | mouse | IgG1; kappa | 74-22-15 | HB-142 |
| Macrophage, pig | mouse | IgG2b; kappa | 74-22-15A | HB-142.1 |
| Macrophage, pig | mouse | IgM; kappa | 76-5-28 | HB-153 |
| Macrophage, pig | mouse | IgM; kappa | 76-6-7 | HB-141 |
| MAdCAM-1, mouse | rat/mouse | IgG2a | MECA-367 | HB-9478^{†} |
| MAdCAM-1, mouse | rat/mouse | IgG2a | MECA-89 | HB-292 |
| Malignant cultured cells, human | mouse | IgM | B5 NIH | HB-10569^{†} |
| Mammalian H-Y antigen | mouse | IgM; kappa | HY3-11.27 | HB-8116 |
| Mammalian sperm acrosomal vesicle | mouse | IgG1 | HS-21 (subclone 1H3) | HB-255 |
| Mammalian splicing factor (SC35) | mouse | IgG1 | anti-SC35 | CRL-2031 |
| Mammary carcinoma cell line, human | mouse | | UCD/AB 6.01 | HB-8693^{†} |
| Mammary carcinoma cell line, human | mouse | | UCD/AB 6.11 | HB-8458^{†} |
| Mammary tumor cell cytoplasmic antigen, human | mouse | IgG1 | 3B18 | HB-8654^{†} |
| Mammary tumor cells, human | mouse | IgM | B25.2 | HB-8107^{†} |
| Mammary tumor cells, human | mouse | IgG1 | B38.1 | HB-8110^{†} |
| Mammary tumor cells, human | mouse | IgG1 | B6.2 | HB-8106^{†} |
| Mammary tumor cells, human | mouse | IgG1 | B72.3 | HB-8108^{†} |
| Medullary thymic epithelium, mouse | rat/mouse | IgM | MD2 | HB-229 |
| Melanoma associated antigens, human | mouse | IgG | WI-MN-1 | HB-8672^{†} |
| Melanoma cell line, human (M-1 antigen system) | mouse | IgG1 | LI 27 | HB-8437^{†} |
| Melanoma cell line, human (M-11 antigen system) | mouse | IgG2a | AL 1-27 | HB-8441^{†} |
| Melanoma cell line, human (M-12 antigen system) | mouse | IgG1 | LI 66 | HB-8442^{†} |
| Melanoma cell line, human (M-13 antigen system) | mouse | IgG1 | E 20 | HB-8443^{†} |
| Melanoma cell line, human (M-16 antigen system) | mouse | IgM | K 114 | HB-8444^{†} |
| Melanoma cell line, human (M-18 antigen system) | mouse | IgG3 | R₂₄ | HB-8445^{†} |
| Melanoma cell line, human (M-19 antigen system) | mouse | IgG1 | L235 | HB-8446^{†} |
| Melanoma cell line, human (M-20 antigen system) | mouse | IgG2a | L101 | HB-8447^{†} |
| Melanoma cell line, human (M-23 antigen system) | mouse | IgG1; kappa | L230 | HB-8448^{†} |
| Melanoma cell line, human (M-26 antigen system) | mouse | IgG1 | A123 | HB-8451^{†} |
| Melanoma cell line, human (M-27 antigen system) | mouse | IgM | A124 | HB-8452^{†} |
| Melanoma cell line, human (M-28 antigen system) | mouse | IgG2a | B5 | HB-8453^{†} |
| Melanoma tumor-specific antigen, human | mouse | IgG2a | XMMME-001 | HB-8759^{†} |
| Melanoma tumor-specific antigen, human | mouse | IgG2a | XMMME-002 | HB-8760^{†} |
| Mesothelial and ciliated cell protein, 130 kDa, | mouse | IgM; kappa | anti-130-kDa | CRL-2401 |
| human and rat | | | Mesothelial-Ciliated Cells | |
| Microglobulin, beta-2, human | mouse | IgG2b | BBM.1 | HB-28 |
| Microglobulin, beta-2, human | mouse | IgG1; kappa | L368 | HB-149 |
| Microglobulin, beta-2, rat | mouse | IgG1 | 4C9 | CRL-2437 |
| Monocyte, Fc receptor (high affinity, FcRI), human | mouse | IgG1 | 32.2 | HB-9469^{†} |
| Monocyte, human | mouse | IgG2b | 3C10 | TIB-228 |
| Monocyte, human | mouse | IgG2a; kappa | 4F2C13 | HB-22 |
| Monocyte, human | mouse | IgG1 | 63D3 | HB-44 |
| Monocyte, human | mouse | IgM; kappa | MMA | HB-78 |
| Monocyte-derived neutrophil chemotactic factor, | mouse | IgG1 | EL-NC-1S | HB-9647^{†} |
| human | | | | |
| Mononuclear cells, human | mouse | IgG2b | OKM 1 | CRL-8026 |
| MOPC167 idiotype (V kappa 24) | rat/mouse | IgG1 and IgG2a | 28-6-20 | CRL-2489 |
| Mu heavy chain, human | mouse | IgG1; kappa | DA4-4 | HB-57 |
| Mu heavy chain, mouse | rat/mouse | IgG2b | 331.12 | TIB-129 |
| Multidrug resistance protein (MRP) | mouse | IgG1 | QCRL-1 | HB-11765^{†} |
| Multidrug resistance protein (MRP) | mouse | IgG2a | QCRL-3 | HB-11766^{†} |
| Muscle, heart | mouse | IgG2a; kappa | 356-1 | HB-181 |
| myb (c-myb), chicken | mouse | IgG2b | MYB 2-3.76 | CRL-1728 |
| myb (c-myb), chicken | mouse | IgG2b | MYB 2-37.63 | CRL-1726 |
| myb (c-myb), chicken | mouse | IgG1 | MYB 2-7.77 | CRL-1724 |
| myb (v-myb) | mouse | IgG2b | MYB 2-3.76 | CRL-1728 |
| myb (v-myb) | mouse | IgG2b | MYB 2-37.63 | CRL-1726 |
| myb (v-myb) | mouse | IgG1 | MYB 2-7.77 | CRL-1724 |
| myc (c-myc) protein, human | mouse | IgG1 | MYC 1-9E10.2 | CRL-1729 |
| myc (c-myc) protein, human | mouse | IgG1 | MYC CT 14-G4.3 | CRL-1727 |
| myc (c-myc) protein, human | mouse | IgG1 | MYC CT 9-B7.3 | CRL-1725 |
| Myeloid cell antigen, human | mouse | IgG1; kappa | Anti-My-10 clone | HB-8483^{†} |
| | | | 28/8/8/14/4 | |
| Myeloid leukemia (CD33), human | mouse | IgG2a | M195 | HB-10306^{†} |
| Myocardium | mouse | IgG2a; kappa | 356-1 | HB-181 |
| Myosin heavy chain, adult, human | mouse | IgG2a | A4.1025 | CRL-2044 |
| Myosin heavy chain, adult, slow, human and rodent | mouse | IgM | A4.840 | CRL-2043 |
| Myosin heavy chain, cardiac alpha, rat | mouse | IgG2b | BA-G5 | HB-276 |
| Myosin heavy chain, embryonic, human and rodent | mouse | IgG1 | F1.652 | CRL-2039 |
| Myosin heavy chain, embryonic, rat | mouse | IgG1 | BF-45 | HB-278 |
| Myosin heavy chain, embryonic, rat | mouse | IgG1 | BF-B6 | HB-279 |
| Myosin heavy chain, fast Ila, human and rodent | mouse | IgG1 | N2.261 | CRL-2047 |
| Myosin heavy chain, fast Ila, rodent | mouse | IgG1 | A4.74 | CRL-2041 |
| Myosin heavy chain, fast Ila, rodent | mouse | IgM | N1.551 | CRL-2040 |
| Myosin heavy chain, fast, human | mouse | IgG1 | A4.74 | CRL-2041 |
| Myosin heavy chain, neonatal and adult fast, human | mouse | IgM | N3.36 | CRL-2042 |
| Myosin heavy chain, slow, human and rodent | mouse | IgG1 | A4.951 | CRL-2046 |
| Myosin heavy chain, slow, human and rodent | mouse | IgG1 | N2.261 | CRL-2047 |
| Myosin heavy chain, type 1, rat | mouse | IgG1 | BA-D5 | HB-287 |
| Myosin heavy chain, type 2A, rat | mouse | IgG1 | SC-71 | HB-277 |
| Myosin heavy chain, type 2B, rat | mouse | IgM | BF-F3 | HB-283 |
| NAP-1 (neutrophil attractant/activation protein 1) | mouse | IgG1 | EL-NC-1S | HB-9647^{†} |
| Nerve growth factor (NGF) receptor, primate | mouse | IgG1 | 200-3-G6-4 (20.4) | HB-8737^{†} |
| Neuroblastoma, human | mouse | IgM | PI 153/3 | TIB-198 |
| Neutrophil attractant/activation protein 1, human | mouse | IgG1 | EL-NC-1S | HB-9647^{†} |
| Neutrophils, mouse | rat/mouse | IgM | J11d.2 | TIB-183 |
| nG4m(b) isoallotope, human | mouse | IgG1 | HP6016 | CRL-1787 |
| Nicotinic acetylcholine receptor, Torpedo californica | mouse | IgG1 | 88B | CRL-1967 |
| NK cell antigen, mouse (LGL-1) | rat/mouse | IgG2a | 4D11 | HB-240 |
| NK cell target ligand on NC-37 cells | mouse | IgM | 18C2.8.3 | HB-9571^{†} |
| NK cell target ligand on NC-37 cells | mouse | IgM | 7C6.5.4 | HB-9574^{†} |
| NK cells, human | mouse | IgM; kappa | HNK-1 | TIB-200 |
| NK cells, mouse | mouse | IgG2a | PK136 | HB-191 |
| Non-small cell lung carcinoma (NSCLC), human | mouse | IgG1 | L18 | HB-8628^{†} |
| Non-small cell lung carcinoma (NSCLC), human | mouse | IgM | L5 | HB-8627^{†} |
| O-antigen, Escherichia coli O157 | mouse | IgM | MARC 29F8 | CRL-2508 |
| O-antigen, Escherichia coli O157 | mouse | IgM | MARC S5 | CRL-2507 |
| OKT-10 like molecule, human | mouse | IgG1 | THB-7 | HB-136 |
| o-Phosphotyrosine | mouse | IgG1; kappa | 2G8.D6 | HB-8190^{†} |
| Ornithine decarboxylase (ODC), mouse | mouse | IgM | B11 | HB-8372^{†} |
| Ovarian carcinoma cell line (2774), human | mouse | IgG1 | ME195 | HB-8431^{†} |
| Ovarian carcinoma cell line (2774), human | mouse | IgG2a | MF 116 | HB-8411^{†} |
| Ovarian carcinoma cell lines, human | mouse | IgM | MH55 | HB-8412^{†} |
| Ovarian carcinoma, human | mouse | IgG2a; IgG2b | OVB-3 | HB-9147^{†} |
| Oxysterol binding protein | mouse | IgG2a | IgG-B16 | CRL-1899 |
| Oxysterol binding protein (OSBP), rabbit | mouse | IgG1 | IgG-11H9 | CRL-2213 |
| Oxytocin-neurophysin (NP-OT), rat | mouse | IgG2b; kappa | PS 38 | CRL-1950 |
| Oxytocin-neurophysin (NP-OT), rat | mouse | IgG2b; kappa | PS 60 | CRL-1800 |
| Oxytocin-neurophysin (NP-OT), rat | mouse | IgG2a; kappa | PS 67 | CRL-1797 |
| p12 gag protein of murine leukemia viruses (MuLV) | mouse | IgG2b | 548 | CRL-1890 |
| p15 gag protein of murine leukemia viruses (MuLV) | mouse | IgG2b | 34 | CRL-1889 |
| p15E env protein of murine leukemia viruses (MuLV) | mouse | IgG3 | 372 | CRL-1893 |
| p30 gag protein of murine leukemia virus (MuLV) | rat/mouse | IgG1 | R187 | CRL-1912 |
| Parainfluenzavirus type 3, fusion glycoprotein (F), | mouse | IgG1 | 9-4-3 | HB-8935^{†} |
| human | | | | |
| Parainfluenzavirus type 3, hemagglutinin (HN), | mouse | IgG2a | 13-5-9-6-2 | HB-8934^{†} |
| human | | | | |
| Paramyosin, Schistosoma mansoni | mouse | IgG2a | MBL-Sm-1A6 | HB-194 |
| Paramyosin, Schistosoma mansoni | mouse | IgG2a | MBL-Sm-4B1 | HB-193 |
| Pasturella multocida type D dermonecrotic toxin | mouse | IgG1 | 1B2A3 | CRL-1965 |
| PDGF B, v-sis form | mouse | IgG2a | 116 | HB-9367^{†} |
| PDGF B, v-sis form | mouse | IgG2b | 232 | HB-9372^{†} |
| PDGF B, v-sis form | mouse | IgG1 | 52 | HB-9361^{†} |
| Peptidoglycan, bacterial | mouse | IgG3; kappa | 15B2 | HB-8510^{†} |
| Peptidoglycan, bacterial | mouse | IgG1; kappa | 3C11 | HB-8511^{†} |
| Peptidoglycan, bacterial | mouse | IgM; kappa | 3F6 | HB-8512^{†} |
| Peptidoglycan, bacterial | mouse | IgM; lambda | 3G3 | HB-8516^{†} |
| Periostin | mouse | IgG1; kappa | 5H8 | CRL-2646 |
| PETA-3 (CD151) | mouse | IgG1 | 41-2 | CRL-2695 |
| PETA-3 (CD151) | mouse | IgG1 | 50-6 | CRL-2696 |
| Peyer's patch endothelial cells, human | mouse | IgG2a | Hermes-3 | HB-9480^{†} |
| Peyer's patch endothelial cells, mouse | rat/mouse | IgG2a | MECA-367 | HB-9478^{†} |
| P-glycoprotein, human | mouse | IgG2b | 443-17F9-1C6 | CRL-2694 |
| Pgp-1 glycoprotein, mouse | rat/mouse | IgG2b | IM7.8.1 | TIB-235 |
| Pgp-1, mouse | rat/mouse | IgG1 | KM114 | TIB-242 |
| Pgp-1, mouse | rat/mouse | IgG1 | KM201 | TIB-240 |
| Pgp-1, mouse | rat/mouse | IgG2a | KM703 | CRL-1896 |
| Pgp-1, mouse | rat/mouse | IgG2a | KM81 | TIB-241 |
| Phenylarsonate | mouse | IgE | SE-1.3 | HB-137 |
| Phosphatidylinositol 4-kinase, type II, bovine | mouse | IgG1; kappa | 4C5G | CRL-2538 |
| Phosphotyrosine | mouse | IgG1; kappa | 2G8.D6 | HB-8190^{†} |
| Phosphotyrosine | mouse | IgG3; kappa | FB2 | CRL-1891 |
| Phosphotyrosine | mouse | IgG1 | P-tyr-1 | CRL-1955 |
| Plasmodium falciparum merozoite antigen | mouse | IgG2b | MAb 5.2 | HB-9148 |
| Platelet glycoprotein GPIIIa, human | mouse | IgG1; kappa | LK-4 | CRL-2345 |
| Platelet-derived growth factor B chain (PDGF B, | mouse | IgG2a | 116 | HB-9367^{†} |
| v-sis form) | | | | |
| Platelet-derived growth factor B chain (PDGF B, | mouse | IgG2b | 232 | HB-9372^{†} |
| v-sis form) | | | | |
| Platelet-derived growth factor B chain (PDGF B, | mouse | IgG1 | 52 | HB-9361^{†} |
| v-sis form) | | | | |
| Platelets, human | mouse | IgG1 | 7E3 | HB-8832^{†} |
| p-nitroanaline amide derivatives | mouse | IgG1 | P3 6D4 (SCRF 43.1) | HB-9168^{†} |
| p-nitroanaline amide derivatives | mouse | IgG1 | P3 8D2 (SCRF 43.1) | HB-9169^{†} |
| p-nitroanaline amide derivatives | mouse | IgG1 | QPN1 12C9 (SCRF 43.2) | HB-9500^{†} |
| p-nitroanaline amide derivatives | mouse | IgG1 | QPN1 22F5 (SCRF 43.2) | HB-9509^{†} |
| Polypeptide, synthetic | mouse | IgM | 7C8 | HB-8465^{†} |
| Polypeptide, synthetic, Asp-Tyr-Lys-Asp-Asp-Asp- | mouse | IgG2b | 4E11 | HB-9259^{†} |
| Asp-Lys | | | | |
| Polypyrimidine tract binding protein (PTB) | mouse | IgG2b; kappa | mAb BB7 | CRL-2501 |
| Pig parvovirus (PPV) | mouse | IgG1 | 3C9-D11-H11 | CRL-1745 |
| Primate tissue, normal | mouse | IgM | B5 NIH | HB-10569^{†} |
| Prostate antigen (PA), human | mouse | IgM; kappa | F5-A-1/22.8.13 | HB-8051^{†} |
| Prostate antigen (PA), human | mouse | IgG1 | RLSD06 | HB-8527^{†} |
| Prostate antigen (PA), human | mouse | IgG1 | RLSD09 | HB-8525^{†} |
| Prostate cancer antigen, human | mouse | IgG1 | 7E11C5 | HB-10494^{†} |
| Prostate cancer, human | mouse | IgG3 | P25.48 | HB-9119^{†} |
| Prostate epithelial cells | mouse/mouse | IgG1 | Prost 410 | HB-11426^{†} |
| Prothrombin, abnormal, human | mouse | IgG1 | JO1-1 | HB-8638^{†} |
| P-selectin, human | mouse | IgG1 | WAPS 12.2 | HB-299 |
| Pseudomonas aeruginosa (flagella type b) | human | | 20H11 | CRL-9300^{†} |
| Pseudomonas aeruginosa lipopolysaccharide (LPS) | human | IgM | C5B7 | CRL-8753^{†} |
| Fisher immunotype 1 (IATS type 6) | | | | |
| Pseudomonas aeruginosa lipopolysaccharide (LPS) | human | IgM | 9D10 | CRL-8752^{†} |
| Fisher immunotype 4 (IATS type 1) | | | | |
| Pseudomonas aeruginosa lipopolysaccharide (LPS) | human | IgM | 8E7 | CRL-8795^{†} |
| Fisher immunotype 7 | | | | |
| Pseudorabies virus (PRV) | mouse | IgG2b | 3G9F3 | CRL-1843 |
| Pseudorabies virus (PRV) | mouse | IgG2b | 6D8MB4 | CRL-1842 |
| RAN-2 (rat neural antigen-2) | mouse | IgG2a | Ran-2 | TIB-119 |
| ras (c-ras) protein, p21 | rat | IgG2a | Y13-238 | CRL-1741 |
| ras (c-ras) protein, p21 | rat | IgG1 | Y13-259 | CRL-1742 |
| ras (v-ras K) oncogene peptide, synthetic | mouse | IgG1 and IgG2b | 147-67C6 | CRL-2654 |
| ras (v-ras) protein, p21 | rat | IgG2a | Y13-238 | CRL-1741 |
| ras (v-ras) protein, p21 | rat | IgG1 | Y13-259 | CRL-1742 |
| ras oncogene peptide, synthetic | mouse | IgG1; kappa | 146-03E04 | CRL-2650 |
| ras, H/N, peptide, synthetic | mouse | IgG1; kappa | 142-24E5 | CRL-2649 |
| ras, Ha, p21 | mouse | IgG1 | MX | HB-9158^{†} |
| Rat neural antigen-2 (RAN-2) | mouse | IgG2a | RAN-2 | TIB-119 |
| Receptor, 1,25-dihydroxy vitamin D3, pig | mouse | IgG1 | XVI E6E6G10 | HB-9496^{†} |
| Receptor, acetylcholine, neuronal, chicken | rat/mouse | IgG2a | mAb 270 | HB-189 |
| Receptor, acetylcholine, neuronal, rat | rat/mouse | IgG2a | mAb 270 | HB-189 |
| Receptor, CD28, mouse | hamster/mouse | IgG | PV1 | HB-12352^{†} |
| Receptor, complement, type 3 (CR3), mouse | rat/mouse | IgG2b | 5C6 Clone 1 | CRL-1969 |
| Receptor, Coxsackievirus-adenovirus, human | mouse | IgG1 | RmcB | CRL-2379 |
| Receptor, epidermal growth factor (EGF) | mouse | IgG1 | 225 | HB-8508^{†} |
| Receptor, epidermal growth factor (EGF) | mouse | IgG1 | 455 | HB-8507^{†} |
| Receptor, epidermal growth factor (EGF) | mouse | IgG2a | 528 | HB-8509^{†} |
| Receptor, epidermal growth factor (EGF) | mouse | IgG | 579 | HB-8506^{†} |
| Receptor, epidermal growth factor (EGF), human | mouse | IgG2a; kappa | Mab 108 | HB-9764^{†} |
| Receptor, epidermal growth factor (EGF), human | mouse | IgM | Mab 96 | HB-9763^{†} |
| Receptor, Epstein-Barr virus (EBV) | mouse | IgG2a; kappa | THB-5 | HB-135 |
| Receptor, Fc alpha, human | mouse | IgM | My 43.51 | HB-12128^{†} |
| Receptor, follicle stimulating hormone (FSH), human | mouse | IgG1 | FSHR-18 | CRL-2688 |
| Receptor, insulin, human | mouse | IgG1; kappa | αIR-1 | HB-175 |
| Receptor, insulin, placental, human | mouse | IgG1 | DII 33.1 | CRL-1827 |
| Receptor, interferon gamma, mouse | rat/mouse | IgG2a; kappa | GR-20 | CRL-2024 |
| Receptor, interferon gamma, mouse | rat/mouse | IgA; kappa | GR-96 | CRL-2013 |
| Receptor, interleukin 12 (IL-12), beta 1 subunit, | rat/mouse | IgG2a | HIL12R1.2B10 | CRL-2359 |
| human | | | | |
| Receptor, interleukin 2 (IL-2), human | mouse | IgG1 | 2A3A1H | HB-8555^{†} |
| Receptor, interleukin 2 (IL-2), human | mouse | IgG2a | 7G7B6 | HB-8784^{†} |
| Receptor, interleukin 2 (IL-2), mouse | rat/mouse | IgM; kappa | 7D4 | CRL-1698 |
| Receptor, interleukin 2 (IL-2), mouse | rat/mouse | IgG1 | PC 61 5.3 | TIB-222 |
| Receptor, interleukin 8, type B, human | mouse | IgG2a | 10H2.12.1 | HB-11494^{†} |
| Receptor, interleukin 8, type B, human | mouse | IgG2a | 4D1.5.7 | HB-11495^{†} |
| Receptor, luteinizing hormone/chorionic | mouse | IgG2a | FSHR-323 | CRL-2689 |
| gonadotropic (LH/hCG), human | | | | |
| Receptor, luteinizing hormone/chorionic | mouse | IgG1 | LHR-1055 | CRL-2687 |
| gonadotropic (LH/hCG), human | | | | |
| Receptor, luteinizing hormone/chorionic | mouse | IgG1 | LHR-29 | CRL-2685 |
| gonadotropic (LH/hCG), human | | | | |
| Receptor, luteinizing hormone/chorionic | mouse | IgG1 | LHR-74 | CRL-2686 |
| gonadotropic (LH/hCG), human | | | | |
| Receptor, nerve growth factor (NGF), primate | mouse | IgG1 | 200-3-G6-4 (20.4) | HB-8737^{†} |
| Receptor, stem cell factor (SCF), human | mouse | IgG2a | BA7.3C.9 | HB-10716^{†} |
| Receptor, transferrin, human | mouse | IgG2a | L5.1 | HB-84 |
| Receptor, transferrin, human | mouse | IgG1 | OKT 9 | CRL-8021 |
| Receptor, transferrin, mouse | rat/mouse | IgM | R17 208.2 | TIB-220 |
| Receptor, transferrin, mouse | rat/mouse | IgG2a | R17 217.1.3 | TIB-219 |
| Receptor, vascular endothelial growth factor (VEGF), | rat/mouse | IgG1; kappa | DC101 | HB-11534^{†} |
| mouse | | | | |
| Receptor, very low density lipoprotein (VLDL) | mouse | IgG1 | IgG-6A6 | CRL-2197 |
| Receptor, vitonectin, human | mouse | IgG1 | B6H12.2 | HB-9771^{†} |
| Red blood cells, sheep | mouse | IgM; lambda | N-S.2.1 | TIB-108 |
| Red blood cells, sheep | mouse | IgM; kappa | N-S.4.1 | TIB-110 |
| Red blood cells, sheep | mouse | IgG3; kappa | N-S.7 | TIB-114 |
| Red blood cells, sheep | mouse | IgG2b | N-S.8.1 | TIB-109 |
| Red blood cells, sheep | mouse | IgG2a; kappa | S-S.1 | TIB-111 |
| Red blood cells, sheep | mouse | IgM; kappa | S-S.3 | TIB-112 |
| Renal carcinoma cell lines, human | mouse | IgG1 | ME195 | HB-8431^{†} |
| Renal carcinoma cell lines, human | mouse | IgG2a | MF 116 | HB-8411^{†} |
| Renal cell carcinoma, human | mouse | IgG1 | DAL K20 | CRL-2288 |
| Renal cell carcinoma, human | mouse | IgG1 | DAL K29 | CRL-2291 |
| Renal cell carcinoma, human | mouse | IgG1 | DAL K45 | CRL-2292 |
| Renin, hog | mouse | IgG1 | F32 VIII C4 | CRL-1653 |
| Reovirus type 3, sigma 1 hemagglutinin | mouse | IgG2a | 9BG5 | HB-167 |
| REP-1, human | mouse | IgG1 | IgG-2F1 | CRL-2419 |
| Retinal Muller cell, rat | mouse | IgG2a | RAN-2 | TIB-119 |
| Retinoblastoma, human | mouse | IgM | PI 153/3 | TIB-198 |
| Ricin, A chain (A1 and A2) | mouse | IgG1 | TFTA1 | CRL-1771 |
| Ricin, B chain | mouse | IgG1 | TFTB1 | CRL-1759 |
| RNA-DNA hybrids | mouse | IgG | S9.6 | HB-8730^{†} |
| Saccharide antigen, Gal beta1-3GalNAc (T antigen) | mouse | IgG3; kappa | JAA-F11 | CRL-2381 |
| Saxitoxin binding component of electroplex | mouse | IgG1 | VD-10 | HB-68 |
| membrane | | | | |
| Sca-1, mouse | rat/mouse | IgG2a | E13 161-7 | HB-215 |
| SCAP | mouse | IgG2b | IgG-9D5 | CRL-2347 |
| Schistosoma mansoni surface (cercariae) | mouse | IgA | 129A3/1 | HB-8087^{†} |
| glycoprotein | | | | |
| Schistosoma mansoni surface (cercariae) | mouse | IgG1 | 130C3/2B/8 | HB-8088^{†} |
| glycoprotein | | | | |
| Schistosoma mansoni surface (cercariae) | mouse | IgG1 | 132C4A/4 | HB-8086^{†} |
| glycoprotein | | | | |
| L-Selectin, human | mouse | IgG1 | DREG200 | HB-302 |
| L-Selectin, human | mouse | IgG1 | DREG56 | HB-300 |
| L-Selectin, mouse | rat/mouse | IgG2a | MEL-14 | HB-132 |
| L-Selectin, sheep and bovine | mouse | IgG1 | DU1-29 | HB-263 |
| Sex lethal gene product (Sxl), female specific, | mouse | IgG1 | mSXL 104 | CRL-1953 |
| Drosophila melanogaster | | | | |
| Sex lethal gene product (Sxl), female specific, | mouse | IgG1 | mSXL 114 | CRL-1954 |
| Drosophila melanogaster | | | | |
| Sex lethal gene product (Sxl), female specific, | mouse | IgG1 | mSXL 18 | CRL-1952 |
| Drosophila melanogaster | | | | |
| Sex lethal gene product (Sxl), female specific, | mouse | IgG1 | mSXL 5 | CRL-1951 |
| Drosophila melanogaster | | | | |
| Shiga toxin | mouse | IgG1; kappa | 13C4 | CRL-1794 |
| Shiga-like toxin I (SLTI) | mouse | IgG1; kappa | 13C4 | CRL-1794 |
| Shiga-like toxin II (SLT-II) | mouse | IgG1; kappa | 11E10 | CRL-1907 |
| Shiga-like toxin II (SLT-II) | mouse | IgG2a; kappa | 11F11 | CRL-1908 |
| SLA a, c, d | mouse | IgG2a | 7-34-1 | CRL-1945 |
| SLA ABd (pig histocompatibility antigen) | mouse | IgG2b; kappa | 74-11-10 | HB-139 |
| SR proteins (pre-mRNA splicing factors) | mouse | IgG1 | anti-SR (1H4) | CRL-2383 |
| SR proteins (pre-mRNA splicing factors) | mouse | IgM | MAb104 | CRL-2067 |
| SR proteins, conserved epitope | mouse | IgG1 | 16H3 | CRL-2385 |
| src (v-src) oncogene peptide, synthetic | mouse | IgG1 | 201-45E9 | CRL-2670 |
| src (v-src) oncogene peptide, synthetic | mouse | IgG2a; IgG2b | 203-7D10 | CRL-2651 |
| src/yes oncogene peptide, synthetic | mouse | IgG1 | 202-11A8 | CRL-2669 |
| SREBP cleavage activating protein | mouse | IgG2b | IgG-9D5 | CRL-2347 |
| SREBP-2, hamster | mouse | IgG2b | IgG-7D4 | CRL-2198 |
| SRp20 proteins (pre-mRNA splicing factors) | mouse | IgG1 | anti-SRp20 (7B4) | CRL-2384 |
| Stem cell antigen 1, mouse (Sca-1) | rat/mouse | IgG2a | E13 161-7 | HB-215 |
| Stem cell factor (SCF) receptor, human | mouse | IgG2a | BA7.3C.9 | HB-10716^{†} |
| Stem cells, mesenchymal, human | mouse | IgG1 | SH2 | HB-10743^{†} |
| Stem cells, mesenchymal, human | mouse | IgG2b | SH3 | HB-10744^{†} |
| Stem cells, mesenchymal, human | mouse | IgG1 | SH4 | HB-10745^{†} |
| Sterol regulatory element binding protein (dSREBP), | mouse | IgG1; kappa | IgG-3B2 | CRL-2693 |
| Drosophila melanogaster | | | | |
| Sterol regulatory element binding protein (SREBP), | mouse | IgG1 | IgG-2A4 | CRL-2121 |
| human | | | | |
| Sterol regulatory element binding protein 2 | mouse | IgG2b | IgG-7D4 | CRL-2198 |
| (SREBP-2), hamster | | | | |
| Sterol regulatory element binding protein 2 | mouse | IgG1 | IgG-1C6 | CRL-2224 |
| (SREBP-2), human | | | | |
| Sterol regulatory element binding protein 2 | mouse | IgG1; kappa | IgG-1D2 | CRL-2545 |
| (SREBP-2), human | | | | |
| Streptococcus mutans | mouse | IgG2a; kappa | SWLA1 | HB-12559^{†} |
| Streptococcus mutans | mouse | IgG2a; kappa | SWLA2 | HB-12560^{†} |
| Streptococcus mutans | mouse | | SWLA3 | HB-12558^{†} |
| SV40 T antigen | mouse | IgG2a | PAb 101 | TIB-117 |
| SV40 T antigen | mouse | IgG1 | PAb 100 | TIB-115 |
| SV40 T antigen, N terminal | mouse | IgG2a | PAb 108 | TIB-230 |
| SV40 T antigen, N terminal | mouse | IgG2a | PAb 108 | TIB-230 |
| Swine leucocyte antigen (SLA) | mouse | IgG2a | 7-34-1 | CRL-1945 |
| T antigen, N terminal, SV40 | mouse | IgG2a | PAb 108 | TIB-230 |
| T antigen, non-viral (NVT), mouse | mouse | IgG2b | PAb 122 | TIB-116 |
| T antigen, SV40 | mouse | IgG2a | PAb 101 | TIB-117 |
| T antigen, SV40 | mouse | IgG1 | PAb 100 | TIB-115 |
| T cell (activated), human | mouse | IgG1 | 10D2F6 | HB-11103^{†} |
| T cell (activated), human | mouse | IgG1 | OKT 9 | CRL-8021 |
| T cell antigen receptor (Jurkat cells), human | mouse | IgM; kappa | C305 | CRL-2424 |
| T cell antigen receptor, gamma/delta negative, sheep | mouse | IgG1; kappa | 86D | HB-286 |
| T cell antigen receptor, human, major framework | mouse | IgG1 | (BF1) 8A3.31 | HB-9283^{†} |
| determinant | | | | |
| T cell antigen receptor, human, major framework | mouse | IgG2a | W4F.5B | HB-9282^{†} |
| determinant | | | | |
| T cell antigen receptor, mouse | hamster/mouse | IgG | H57-597 | HB-218 |
| T cell antigen receptor, mouse | rat/mouse | IgG2b | TR 310 | HB-219 |
| T cell precursor, human | mouse | IgG1 | OKT 10 | CRL-8022 |
| T cell receptor, gamma/delta, mouse | hamster/mouse | IgG | UC3-10A6 | CRL-1988 |
| T cell receptor, gamma/delta, mouse | hamster/mouse | IgG | UC7-13D5 | CRL-1989 |
| T cells, cytotoxic, pig | mouse | IgG2a; kappa | 76-2-11 | HB-143 |
| T cells, cytotoxic/suppressor, human | mouse | IgG1 | OKT 5 | CRL-8013 |
| T cells, cytotoxic/suppressor, human | mouse | IgG1 | OKT 5 | CRL-8016 |
| T cells, cytotoxic/suppressor, human | mouse | IgG2a | OKT 8 | CRL-8014 |
| T cells, gamma/delta positive, bovine | mouse | IgG1 | IL-A29 | CRL-1874 |
| T cells, helper/inducer, human | mouse | IgG2b | OKT 4 | CRL-8002 |
| T cells, helper/inducer, mouse | rat/mouse | IgG2b | GK1.5 | TIB-207 |
| T cells, human | mouse | IgM | 2T8-3E10 | HB-8213^{†} |
| T cells, human | mouse | IgM | 3Pt12B8 | HB-8136^{†} |
| T cells, human | mouse | IgG1; kappa | 5E9C11 | HB-21 |
| T cells, human | mouse | IgG1 | OKT 1 | CRL-8000 |
| T cells, human | mouse | IgG1 | OKT 11 | CRL-8027 |
| T cells, human | mouse | IgG2a | OKT 3 | CRL-8001 |
| T cells, human | mouse | IgG1; kappa | T3-3A1 | HB-2 |
| T cells, mouse | mouse | IgM; kappa | 20-10-5S | HB-23 |
| T cells, pig | mouse | IgM; kappa | 76-5-28 | HB-153 |
| T cells, pig | mouse | IgM; kappa | 76-6-7 | HB-141 |
| T cells, rabbit | mouse | IgM | 9AE10 | CRL-1761 |
| T cells, rabbit | mouse | IgG1 | L11/135 | TIB-188 |
| T12 (120 kDa) antigen, human T cells | mouse | IgM | 3Pt12B8 | HB-8136^{†} |
| T15 idiotype | mouse | IgG1; kappa | AB1-2 | HB-33 |
| TAG-72 (tumor-associated glycoprotein), human | mouse | IgG1; kappa | CC 49 | HB-9459^{†} |
| T-B activating molecule (T-BAM), human | mouse | IgG2a | 5c8 | HB-10916^{†} |
| Tetanus toxin | human/mouse | IgG1 | 9F12 | HB-8177^{†} |
| Tetanus toxoid | human | IgG; kappa | SA13 | HB-8501^{†} |
| TGF-beta-2, mouse | mouse | IgG1 | 1D11.16.8 | HB-9849^{†} |
| Theophylline | mouse | IgG1; kappa | 17/14 | HB-8153^{†} |
| Theophylline | mouse | IgG1; kappa | 30/15 | HB-8152^{†} |
| Theophylline | mouse | IgG1; kappa | 61/7 | HB-8154^{†} |
| Thy-1 antigen, human | mouse | IgG1 | K117 | HB-8553^{†} |
| Thy-1 antigen, mouse | mouse | IgM | HO-22-1 | TIB-100 |
| Thy-1 antigen, mouse | rat/mouse | IgG2a | M5/49.4.1 | TIB-238 |
| Thy-1.1 antigen, mouse | mouse | IgM | T11D7e2 | TIB-103 |
| Thy-1.2 antigen, mouse | mouse | IgM | HO-13-4 | TIB-99 |
| Thy-1.2 antigen, mouse | rat/mouse | IgG2b | 30-H12 | TIB-107 |
| Thy-1.2 antigen, mouse | rat/mouse | IgM | J1j.10 | TIB-184 |
| Thymic lymphocyte, human | mouse | IgG1 | OKT 6 | CRL-8020 |
| Thymocyte (E rosette positive), human | mouse | IgG1 | OKT 11 | CRL-8027 |
| Thymocyte, human | mouse | IgG2a | A1G3 | HB-177 |
| Thymocyte, human | mouse | IgG1 | OKT 6 | CRL-8020 |
| Thymocytes, rabbit | mouse | IgM | 9AE10 | CRL-1761 |
| Thymus, cortical epithelium, human | mouse | IgG2b | CDR2 | HB-214 |
| Thymus, cortical epithelium, human | mouse | IgG2 | TE3 | HB-209 |
| Thymus, cortical epithelium, human | mouse | IgM | TE4 | HB-207 |
| Thymus, epithelium, human | mouse | IgM | TE15 | HB-206 |
| Thymus, epithelium, human | mouse | IgG1 | TE16 | HB-210 |
| Thymus, epithelium, human | mouse | IgM | TE19 | HB-211 |
| Thymus, epithelium, human | mouse | IgG2a | TE8 | HB-212 |
| Thymus, human | mouse | IgG1 | TE7 | HB-208 |
| Thyroid stimulating hormone (TSH) receptor alpha | mouse | IgG1 | TSHR-R5T-44 | CRL-2681 |
| subunit, human | | | | |
| Thyroid stimulating hormone (TSH) receptor alpha | mouse | IgG1 | TSHR-T5-51 | CRL-2680 |
| subunit, human | | | | |
| Thyroid stimulating hormone (TSH) receptor alpha | mouse | IgG1 | TSHR-T5U-317 | CRL-2682 |
| subunit, human | | | | |
| Thyroid stimulating hormone (TSH) receptor beta | mouse | IgG1 | TSHR-R5T-34 | CRL-2683 |
| subunit, human | | | | |
| Thyroid stimulating hormone (TSH) receptor beta | mouse | IgG2a | TSHR-T3-365 | CRL-2684 |
| subunit, human | | | | |
| L-Thyroxine (T4,3,5,3′,5′-tetraiodo-L-thyronine) | mouse | IgG1 | T4 Clone 5 (10-0101, | HB-8500^{†} |
| | | | 0062-83) | |
| Ti1b antigen, human | mouse | IgM | 2T8-3E10 | HB-8213^{†} |
| TL antigen, mouse | mouse | IgG2a | I(TL.m9) | HB-131 |
| TL antigen, mouse | rat/mouse | IgG2a | HD168 | HB-252 |
| Transferrin receptor, human | mouse | IgG2a | L5.1 | HB-84 |
| Transferrin receptor, human | mouse | IgG1 | OKT 9 | CRL-8021 |
| Transferrin receptor, mouse | rat/mouse | IgM | R17 208.2 | TIB-220 |
| Transferrin receptor, mouse | rat/mouse | IgG2a | R17 217.1.3 | TIB-219 |
| Transforming growth factor-beta2, mouse | mouse | IgG1 | 1D11.16.8 | HB-9849^{†} |
| Treponema pallidum | mouse | IgM | 1939-3G5 | HB-8133^{†} |
| Treponema pallidum | mouse | IgG1 | 1939-8G2 | HB-8134^{†} |
| Trichinella spiralis | mouse | IgM | 7C₂C₅C₁₂ | HB-8678^{†} |
| Trifucosylated type 2 chain glycolipids | mouse | IgM | FHCR-1-2075/FH5 | HB-8770^{†} |
| 2,4,6-Trinitrophenyl (TNP) | mouse | IgG1 | 1B7.11 | TIB-191 |
| 2,4,6-Trinitrophenyl (TNP) | mouse | IgA | 2F.11.15 | TIB-194 |
| 2,4,6-Trinitrophenyl (TNP) | mouse | IgE (Iga haplotype) | IGEL a2 | TIB-142 |
| 2,4,6-Trinitrophenyl (TNP) | mouse | IgE (Igb haplotype) | IGEL b4 | TIB-141 |
| Trop-1, human | mouse | IgG2a | 162-21.2 | HB-241 |
| Trop-2, human | mouse | IgG1 (Igh-4a | 162-46.2 | HB-187 |
| | | allotype) | | |
| Trophoblasts, human | mouse | IgG1 (Igh-4a | 162-46.2 | HB-187 |
| | | allotype) | | |
| Tubulin, beta, nematode | mouse | IgG | P3D | HB-11129^{†} |
| Tumor, intracellular antigen, human | human/mouse | IgM | Ch13 | HB-8573^{†} |
| Tumor, intracellular antigen, human | human/mouse | IgM | Gr431 | HB-8575^{†} |
| Tumor, intracellular antigen, human | human/mouse | IgM | Te39 | HB-8577^{†} |
| Tumor-associated glycoprotein (TAG-72), human | mouse | IgG1 kappa | CC 49 | HB-9459^{†} |
| Tumors, human | mouse | IgM | B5 NIH | HB-10569^{†} |
| Tumors, neuroectoderm, human | mouse | IgM | PI 153/3 | TIB-198 |
| Uracil DNA glycosylase (UDG), human | mouse | IgM | 37.04.12 | HB-9312^{†} |
| Uracil DNA glycosylase (UDG), human | mouse | IgG | 40.10.09 | HB-9311^{†} |
| Uracil DNA glycosylase (UDG), human | mouse | IgM | 42.08.07 | HB-9313^{†} |
| Uterine carcinoma cell lines, human | mouse | IgM | MH55 | HB-8412^{†} |
| VacA (vacuolating cytotoxin) | mouse | IgG1; kappa | 5E4 | CRL-2635 |
| VacA (vacuolating cytotoxin) | mouse | IgG1; kappa | 5G5 | CRL-2633 |
| VacA (vacuolating cytotoxin) | mouse | IgG1; kappa | B3D | CRL-2634 |
| Vascular cell adhesion molecule 1, human and | mouse | IgG1; kappa | VIII-6G10 | HB-10519^{†} |
| macaque | | | | |
| Vascular cell adhesion molecule 1, mouse | rat/mouse | IgG1; kappa | M/K-1.9 | CRL-1910 |
| Vascular cell adhesion molecule 1, mouse | rat/mouse | IgG1; kappa | M/K-2.7 | CRL-1909 |
| Vascular endothelial growth factor (VEGF) | rat/mouse | IgG1; kappa | DC101 | HB-11534^{†} |
| receptor-2, mouse | | | | |
| Vasopressin-neurophysin (NP-AVP), rat | mouse | IgG2b; kappa | PS 41 | CRL-1799 |
| Vasopressin-neurophysin (NP-AVP), rat | mouse | IgG2b; kappa | PS 45 | CRL-1798 |
| VCAM-1, human and macaque | mouse | IgG1; kappa | VIII-6G10 | HB-10519^{†} |
| VCAM-1, mouse | rat/mouse | IgG1; kappa | M/K-1.9 | CRL-1910 |
| VCAM-1, mouse | rat/mouse | IgG1; kappa | M/K-2.7 | CRL-1909 |
| Very late antigen 1 (VLA-1) alpha, human | mouse | IgG1 | TS2/7.1.1 | HB-245 |
| Very late antigen 1 (VLA-1) beta, human | mouse | IgG1 | TS2/16.2.1 | HB-243 |
| Very late antigen 4 (VLA-4), mouse | rat/mouse | IgG2b | R1-2 | HB-227 |
| Very late antigen 4 (VLA-4), sheep | mouse | IgG2b | FW3-218-1 | HB-261 |
| Very low density lipoprotein (VLDL) receptor | mouse | IgG1 | IgG-6A6 | CRL-2197 |
| Vesicular stomatitis virus surface glycoprotein | mouse | IgG1 | 11-Hybridoma | CRL-2700 |
| Vitamin B6 | mouse | IgG1 | E6(2)2 | HB-8172^{†} |
| Vitamin D3 receptor, pig | mouse | IgG1 | XVI E6E6G10 | HB-9496^{†} |
| Vitonectin receptor (VnR), human | mouse | IgG1 | B6H12.2 | HB-9771^{†} |
| Vulva, cancer | human | IgG4 | VLN3G2 | HB-8636^{†} |
| Vulva, cancer | human | IgG | VLN6H2 | HB-8633^{†} |
| WC1, bovine | mouse | IgG2a | CC15 | HB-265 |
| WC1, bovine | mouse | IgG1 | CC39 | HB-274 |
| WC1, bovine | mouse | IgG1 | IL-A29 | CRL-1874 |
| WC3 bovine B cell antigen (BoWC3) | mouse | IgG1 | CC21 | HB-288 |
| WC4, bovine | mouse | IgG1 | CC55 | HB-282 |
| WC4, bovine | mouse | IgG1 | CC57 | HB-268 |
| Yellow fever virus | mouse | IgG2a; kappa | 2D12 | CRL-1689 |
| Yersinia pestis, F1 antigen | mouse | IgA | F1-3G8-1 | HB-192 |
| yes (c-yes) oncogene peptide, synthetic | mouse | IgG1 | 240-13D10 | CRL-2672 |
| ZP1 glycoprotein, mouse | rat/mouse | IgG2a | M1.4 | CRL-2464 |
| ZP2 glycoprotein, human | mouse | IgG1; kappa | H2.8 | CRL-2568 |
| ZP2 glycoprotein, mouse | rat/mouse | IgG2a | IE-3 | CRL-2463 |
| ZP3 glycoprotein, human | mouse | IgG1; kappa | H3.1 | CRL-2569 |
| ZP3 glycoprotein, mouse | rat/mouse | IgG2a | IE-10 | CRL-2462 |

We claim:

1. A cell culture media comprising the following components: basal media, approximately 1-4 mM glutamine, approximately 0.3-2% albumin, approximately 5-15 mg/L insulin, approximately 0.5-9 mg/L transferrin, approximately 5-15 uM ethanolamine, and approximately 1-3% of a purified lipoprotein material.

2. A cell culture media comprising the following components: basal media, sodium selenite, albumin, insulin, transferrin, peptone, fetuin, vitamin E and a purified lipoprotein material.

3. The cell culture media of claim 1 or 2 wherein the purified lipoprotein material is derived from bovine serum or plasma.

4. The cell culture media of claim 1 or 2 wherein the purified lipoprotein material is cholesterol-rich.

5. The cell culture media of claim 1 or 2 wherein the purified lipoprotein material is produced by contacting serum or plasma with a silica adsorbant.

6. A method to culture cells comprising incubating the cells in a culture media consisting essentially of the following components: basal media, glutamine, albumin, insulin, transferrin, ethanolamine, and a purified lipoprotein material.

7. A method to culture cells comprising incubating the cells in a culture media comprising the following components: basal media, sodium selenite, albumin, insulin, transferrin, peptone, fetuin, vitamin E and a purified lipoprotein material.

8. The method of claim 6 or 7 wherein the purified lipoprotein material is derived from bovine serum or plasma.

9. The method of claim 6 or 7 wherein the purified lipoprotein material is cholesterol-rich.

10. The method of claim 6 or 7 wherein the purified lipoprotein material is produced by contacting serum or plasma with a silica adsorbant.

11. The method of claim 6 wherein the cells produce a peptide.

12. The method of claim 11 wherein the peptide is an antibody.

13. The method of claim 11 wherein the peptide is an immunoglobulin.

14. The method of claim 6 wherein the cells are hybridoma cells.

15. The method of claim 7 wherein the cells are cancer cells.

16. The method of claim 7 wherein the cells are in suspension.

17. The method of claim 7 wherein the cells are adherent.

18. The method of claim 7, wherein the cells are selected from the group consisting of K562 cells, HL-60 cells, Daudi cells, HeLA cells, THP-1 cells, Jurkat cells and EL4 cells.

19. The method of claim 7 wherein the cells are human cells.

20. A method for culturing hybridoma cells in vitro comprising incubating the cells in a cell culture media comprising the following components: basal media, glutamine, albumin, insulin, transferrin, ethanolamine, and a purified lipoprotein material.

21. A cell culture media consisting essentially of the following components: basal media, glutamine, albumin, insulin, transferrin, ethanolamine, and a purified lipoprotein material.
